(19) United States
(12) Patent Application Publication
(10) Pub. No.: US 2015/0050344 A1
(43) Pub. Date: Feb. 19, 2015

(54) COMPOSITIONS AND METHODS FOR UPREGULATING HIPPOCAMPAL PLASTICITY AND HIPPOCAMPUS-DEPENDENT LEARNING AND MEMORY

(71) Applicant: Revalesio Corporation, Tacoma, WA (US)

(72) Inventors: Richard L. Watson, Ruston, WA (US); Anthony B. Wood, Tacoma, WA (US); Gregory J. Archambeau, Puyallup, WA (US); Supurna Ghosh, Sammamish, WA (US)

(21) Appl. No.: 14/339,387

(22) Filed: Jul. 23, 2014

Related U.S. Application Data
(60) Provisional application No. 61/857,306, filed on Jul. 23, 2013.
(60) Provisional application No. 61/888,420, filed on Oct. 8, 2013.
(60) Provisional application No. 61/930,388, filed on Jan. 22, 2014.

(51) Int. Cl. A61K 9/51; A61K 45/06; A61K 33/00

(52) U.S. Cl. CPC A61K 9/51; A61K 33/00; A61K 45/06; A61K 9/5115
USPC 424/489; 424/613; 424/85.6; 424/133.1; 435/375

(57) ABSTRACT

Provided are methods for enhancing hippocampal plasticity and hippocampal-mediated learning and memory, and/or enhancing the synaptic maturation of neurons, and/or optimizing or enhancing neuronal synaptic transmission, and/or enhancing intracellular oxygen delivery or utilization, and/or enhancing ATP synthesis, comprising administration, to a subject in need thereof of a sufficient amount over a sufficient time, of an ionic aqueous solution of charge-stabilized oxygen-containing nanostructures (e.g., nanobubbles) having an average diameter of less than 100 nm (e.g., in at least one subject group selected from but not limited to normal subjects, subjects recovering from neurological trauma (e.g., accidents or injury to the brain, stroke, oxygen deprivation, drowning, and asphyxia), and subjects with learning disorders (e.g., dyslexia, dyscalculia, dysgraphia, dyspraxia (sensory integration disorder), dysphasia/aphasia, auditory processing disorder, non-verbal learning disorder, visual processing disorder, and attention deficit disorder (ADD)).

### CROSS-REFERENCE TO RELATED APPLICATIONS

This application claims the benefit of co-pending U.S. Provisional Patent Application Ser. No. 61/857,306, filed Jul. 23, 2013 and entitled COMPOSITIONS AND METHODS FOR UPREGULATING HIPPOCAMPAL PLASTICITY AND HIPPOCAMPUS-DEPENDENT LEARNING AND MEMORY, U.S. Provisional Patent Application Ser. No. 61/888,420, filed Oct. 8, 2013 and entitled COMPOSITIONS AND METHODS FOR UPREGULATING HIPPOCAMPAL PLASTICITY AND HIPPOCAMPUS-DEPENDENT LEARNING AND MEMORY, and U.S. Provisional Patent Application Ser. No. 61/930,388, filed Jan. 22, 2014 and entitled COMPOSITIONS AND METHODS FOR OPTIMIZING NEURONAL SYNAPTIC TRANSMISSION, all of which are incorporated herein by reference in their entireties.

### FIELD OF THE INVENTION

Particular aspects relate generally to hippocampus-dependent learning and memory, and in more particular aspects to compositions and methods for upregulating hippocampal plasticity and hippocampus-dependent learning and memory in a subject by administering a therapeutic composition comprising a gas-enriched (e.g., oxygen enriched) electrokinetically generated fluid comprising charge-stabilized oxygen-containing nanostructures, as disclosed herein. Additional aspects relate to methods for enhancing the synaptic maturation of neurons by enriching the density and size of dendritic spines (e.g., comprising enhancing at least one of the length of primary axons, the number of collaterals, or the number of tertiary branches). Additional aspects relate generally to neurons and neuronal synaptic transmission, and more particularly to compositions and methods for optimizing or enhancing neuronal synaptic transmission. Further aspects relate to methods for enhancing intracellular oxygen delivery or utilization (particularly in neurons), and methods for enhancing ATP synthesis (e.g., at presynaptic and/or postsynaptic terminals). Additional aspects relate to combination therapies.

### BACKGROUND OF THE INVENTION

Increased calcium influx through ionotropic glutamate receptors and the upregulation of plasticity-associated molecules in hippocampal neurons are two important events in the process of hippocampus-dependent spatial learning and memory.

Additionally, increased density of dendritic spines and enhanced synaptic transmission through ionotropic glutamate receptors are important events of synaptic plasticity and eventually in the process of hippocampus-dependent spatial learning and memory.

Hippocampal neuron function is also implicated in neurodegenerative disease. Alzheimer's disease (AD), for example, is the most common neurodegenerative disorder in the aged population characterized by impairments in memory and cognition. An extensive loss of hippocampal neurons (1) is the hallmark of this disease. The death of hippocampal neurons is often associated with and the strong downregulation of many functional genes (2) involved in ion conductance (3, 4), synapse formation (5), dendritic arborization (6), long term potentiation (7, 8), and long term depression (8, 9). Impaired calcium influx through ionotropic glutamate receptors including NMDA and AMPA receptors is directly linked to the loss of hippocampal learning and memory (10). Analysis of postmortem AD brains showed that expression of NMDA subunits including NR1, NR2A, and NR2B was altered in susceptible brain regions including hippocampus (11). Downregulation of immediate early genes (IEGs) (12) including arc, zif-268, homer-1, c-fos and inhibition of synaptic genes (13-15) including psd-95, synpo, adam-10 was also reported to be downregulated in AD brain. In addition, oxidative (16) and nitrosylative (17, 18) damages in different hippocampal proteins also have been implicated in the loss of function and eventually death of hippocampal neurons. Many pharmacological compounds have been tested in the treatment of these progressive neurodegenerative diseases including cholinesterase inhibitors and memantine, but most of them generate several side effects, perhaps because of lower metabolic activities of elderly population, or perhaps because of toxicity because they are metabolized.

Aside from treating neurodegenerative diseases, however, there is a pronounced need in the art for compositions and methods to enhance neuroplasticity and learning in the general population (in addition to enhancing neuroplasticity and learning in the context of neurodegenerative diseases).

### SUMMARY OF THE INVENTION

According to particular aspects, the disclosed electrokinetically-altered fluids (e.g., RNS60) control or modulate (e.g., increase or enhance) the synaptic plasticity of hippocampal neurons by inducing calcium influx via NMDA- and AMPA-sensitive ionotropic glutamate receptors. RNS60, but neither NS nor PNS, stimulates the expression of NR2A, NR2B subunits NMDA and GluR1 subunit of AMPA receptors along with other plasticity-associated molecules including Arc, PSD95, and CREB.

Particular aspects, therefore, provide a method for enhancing hippocampal plasticity and hippocampus-dependent learning and/or memory, comprising administering to a subject in need thereof a therapeutically effective amount of an electrokinetically altered aqueous fluid comprising an ionic aqueous solution of charge-stabilized oxygen-containing nanostructures (e.g., nanobubbles) predominantly having an average diameter of less than about 100 nanometers and stably configured in the ionic aqueous fluid in an amount sufficient for enhancing hippocampal plasticity and hippocampus-dependent learning and/or memory in the subject.

Particular aspects, therefore, provide a method for enhancing hippocampal-mediated learning and memory, comprising administering to a subject in need thereof a therapeutically effective amount of an ionic aqueous solution of charge-stabilized oxygen-containing nanostructures having an average diameter of less than 100 nanometers for enhancing hippocampal-mediated learning and memory in the subject.

In particular aspects of the methods, the ionic aqueous solution comprises dissolved oxygen in an amount of at least 8 ppm, at least 15, ppm, at least 25 ppm, at least 30 ppm, at least 40 ppm, at least 50 ppm, or at least 60 ppm oxygen at atmospheric pressure. In particular aspects of the methods, the percentage of dissolved oxygen molecules present in the solution as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%; 60%; 65%; 70%; 75%; 80%; 85%; 90%; and 95%. In particular aspects of the methods, the amount of dissolved oxygen present in charge-stabilized oxygen-containing nanostructures is at least 8 ppm, at least 15, ppm, at least 20 ppm, at least 25 ppm, at least 30 ppm, at least 40 ppm, at least 50 ppm, or at least 60 ppm oxygen at atmospheric pressure. In particular aspects of the methods, the majority of the dissolved oxygen is present in the charge-stabilized oxygen-containing nanostructures. In particular aspects of the methods, the charge-stabilized oxygen-containing nanostructures have an average diameter of less than a size selected from the group consisting of: 90 nm; 80 nm; 70 nm; 60 nm; 50 nm; 40 nm; 30 nm; 20 nm; 10 nm; and less than 5 nm. In particular aspects of the methods, the ionic aqueous solution comprises a saline solution. In particular aspects of the methods, the solution is superoxygenated.

In particular aspects of the methods, the charge-stabilized oxygen-containing nanostructures comprise charge-stabilized oxygen-containing nanobubbles having an average diameter of less than 100 nanometers.

In particular aspects of the methods comprise modulating at least one of cellular membrane potential and cellular membrane conductivity in hippocampal cells of the subject.

In particular aspects of the methods, enhancing learning and/or memory, comprises enhancing learning and/or memory in at least one group selected from the group consisting of normal subjects, subject recovering from neurological trauma, and subjects with learning disorders. In particular aspects of the methods, the learning disorder comprises one selected from the group consisting of, dyslexia, dyscalculia, dysgraphia, dyspraxia (sensory integration disorder), dysphasia/aphasia, auditory processing disorder, non-verbal learning disorder, visual processing disorder, and attention deficit disorder (ADD). In particular aspects of the methods, neurological trauma comprises at least one of accidents or injury to the brain, stroke, oxygen deprivation, drowning, and asphyxia.

In particular aspects of the methods, administration promotes modulating (e.g., upregulating, in hippocampal neurons, of expression, amount or activity levels of at least one neuronal plasticity protein selected from the group consisting of NR2A and/or NR2B subunits NMDA receptors, GluR1 (glur1) subunit of AMPA receptors, Arc (arc), PSD95, CREB (creb): IEGs including arc, zif-268, and c-fos; NMDA receptor subunits including nr1, nr2a, nr2b, and nr2c; AMPA receptor subunit glur1; neurotrophic factors and their receptors including bdnf, nt3, nt5, and ntrk2; adenylate cyclases (adcy1 and adcy8); camk2a, akt1; ADAM-10, Synpo and homer-1.

In particular aspects of the methods, administration promotes modulating (e.g., downregulating expression, amount or activity levels of at least one protein selected from the group consisting of Gria2, Ppp1ca, Ppp2ca, and Ppp3ca, proteins encoded by genes known to support long-term depression.

Particular aspects of the methods comprise combination therapy, wherein at least one additional therapeutic agent is administered to the patient. In particular aspects of the methods, the at least one additional therapeutic agent is selected from the group consisting of: glatiramer acetate, interferon-β, mitoxantrone, natalizumab, inhibitors of MMPs including inhibitor of MMP-9 and MMP-2, short-acting β₂-agonists, long-acting β₂-agonists, anticholinergics, corticosteroids, systemic corticosteroids, mast cell stabilizers, leukotriene modifiers, methylxanthines, β₂-agonists, albuterol, levalbuterol, pirbuterol, artformoterol, formoterol, salmeterol, anticholinergics including ipratropium and tiotropium; corticosteroids including beclomethasone, budesonide, flunisolide, fluticasone, mometasone, triamcinolone, methyprednisolone, prednisolone, prednisone; leukotriene modifiers including montelukast, zafirlukast, and zileuton; mast cell stabilizers including cromolyn and nedocromil; methylxanthines including theophylline; combination drugs including ipratropium and albuterol, fluticasone and salmeterol, budesonide and formoterol; antihistamines including hydroxyzine, diphenhydramine, loratadine, cetirizine, and hydrocortisone; immune system modulating drugs including tacrolimus and pimecrolimus; cyclosporine; azathioprine; mycophenolatemofetil; and combinations thereof. In particular aspects of the methods, the at least one additional therapeutic agent is an anti-inflammatory agent.

In particular aspects of the methods, modulation of at least one of cellular membrane potential and cellular membrane conductivity comprises modulating at least one of cellular membrane structure or function comprising modulation of at least one of an amount, conformation, activity, ligand binding activity and/or a catalytic activity of a membrane associated protein. In particular aspects of the methods, the membrane associated protein comprises at least one selected from the group consisting of receptors, ion channel proteins, intracellular attachment proteins, cellular adhesion proteins, and integrins. In particular aspects of the methods, the receptor comprises a transmembrane receptor. In particular aspects of the methods, modulating cellular membrane conductivity comprises modulating whole-cell conductance. In particular aspects of the methods, modulating whole-cell conductance comprises modulating at least one voltage-dependent contribution of the whole-cell conductance.

In particular aspects of the methods, modulation of at least one of cellular membrane potential and cellular membrane conductivity comprises modulating a calcium dependent cellular messaging pathway or system. Particular aspects of the methods comprise modulating calcium influx through ionotropic glutamate receptors (e.g., comprises at least one NMDA and/or AMPA receptor).

In particular aspects of the methods, modulation of at least one of cellular membrane potential and cellular membrane conductivity comprises modulating intracellular signal transduction comprising modulation of phospholipase C activity.

In particular aspects of the methods, modulation of at least one of cellular membrane potential and cellular membrane conductivity comprises modulating intracellular signal transduction comprising modulation of adenylate cyclase (AC) activity.

Particular aspects of the methods comprise administration to a cell network or layer, and further comprising modulation of an intercellular junction therein.

In particular aspects of the methods, the solution comprises at least one of a form of solvated electrons, and electrokinetically modified or charged oxygen species. In particular aspects of the methods, the form of solvated electrons or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm. In particular aspects of the methods, the electrokinetically altered oxygenated aqueous fluid comprises solvated electrons stabilized, at least in part, by molecular oxygen.

In particular aspects of the methods, the ability of the solution to modulate of at least one of cellular membrane potential and cellular membrane conductivity persists for at least two, at least three, at least four, at least five, at least 6, at least 12 months, or longer periods, in a closed gas-tight container.

In particular aspects of the methods, treating/administering comprises administration by at least one of topical, inhalation, intranasal, oral, intravenous (IV) and intraperitoneal (IP).

In particular aspects of the methods, the charge-stabilized oxygen-containing nanostructures are formed in a solution comprising at least one salt or ion from Tables 1 and 2 disclosed herein.

In particular aspects of the methods, the subject is a mammal, preferably a human.

Additional aspects provide a method for enhancing the synaptic maturation of neurons by enriching the density and size of dendritic spines, comprising administering to a neuron or subject in need thereof a therapeutically effective amount of an ionic aqueous solution of charge-stabilized oxygen-containing nanostructures having an average diameter of less than 100 nanometers sufficient for enhancing the synaptic maturation of neurons by enriching the density and size of dendritic spines. Particular embodiments comprise enhancing at least one of the length of primary axons, the number of collaterals, or the number of tertiary branches. In certain aspects, the ionic aqueous solution comprises dissolved oxygen in an amount of at least 8 ppm, at least 15, ppm, at least 25 ppm, at least 30 ppm, at least 40 ppm, at least 50 ppm, or at least 60 ppm oxygen at atmospheric pressure. In certain aspects, the percentage of dissolved oxygen molecules present in the solution as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%; 60%; 65%; 70%; 75%; 80%; 85%; 90%; and 95%. In certain aspects, the amount of dissolved oxygen present in charge-stabilized oxygen-containing nanostructures is at least 8 ppm, at least 15, ppm, at least 20 ppm, at least 25 ppm, at least 30 ppm, at least 40 ppm, at least 50 ppm, or at least 60 ppm oxygen at atmospheric pressure. In certain aspects, the majority of the dissolved oxygen is present in the charge-stabilized oxygen-containing nanostructures. In certain aspects, the charge-stabilized oxygen-containing nanostructures have an average diameter of less than a size selected from the group consisting of: 90 nm; 80 nm; 70 nm; 60 nm; 50 nm; 40 nm; 30 nm; 20 nm; 10 nm; and less than 5 nm. In certain aspects, the ionic aqueous solution comprises a saline solution. In certain aspects, the solution is superoxygenated. In certain aspects, the neurons are hippocampal neurons. Certain aspects comprise administration to neurons ex vivo, in vivo or in vitro.

In certain aspects, the charge-stabilized oxygen-containing nanostructures comprise charge-stabilized oxygen-containing nanobubbles having an average diameter of less than 100 nanometers.

Further aspects comprise methods for maintaining, growing or enhancing the synaptic maturation of neurons in culture.

Yet further aspects relate to optimizing or enhancing neuronal synaptic transmission, and/or for enhancing intracellular oxygen delivery or utilization (particularly in neurons), and methods for enhancing ATP synthesis (e.g., at presynaptic and/or postsynaptic terminals).

Determining the biological variables that control both electrical and chemical synaptic transmission between nerve cells, or between nerve terminals and muscular or glandular systems, has been a very significant area of physiological exploration over the decades. Chemical synaptic transmission has had the added attraction of addressing both the transmission gain of the event, as well as the excitatory or inhibitory nature of the junction and its activity-dependent potentiation or depression.

Provided are methods for optimizing or enhancing neurotransmission (neuronal synaptic transmission), comprising administrating an electrokinetically-altered ionic aqueous solution comprising charge-stabilized oxygen-containing nanostructures (e.g., oxygen-containing nanobubbles) having an average diameter of less than 100 nm in an amount and for a time period sufficient for modulating at least one presynaptic and/or postsynaptic response.

Additional aspects provide a method for optimizing or enhancing neurotransmission, comprising contacting neurons with, or administrating to a subject having neurons, an electrokinetically-altered ionic aqueous solution comprising charge-stabilized oxygen-containing nanostructures having an average diameter of less than 100 nm in an amount and for a time period sufficient for enhancing intracellular oxygen delivery or utilization, wherein a method for optimizing neuronal synaptic transmission is afforded.

Further aspects provide a method for enhancing intracellular oxygen delivery or utilization, comprising contacting cells with, or administrating to a subject having cells, an electrokinetically-altered ionic aqueous solution comprising charge-stabilized oxygen-containing nanostructures having an average diameter of less than 100 nm in an amount and for a time period sufficient for enhancing intracellular oxygen delivery or utilization in the cells.

For the above methods for optimizing or enhancing neurotransmission, representative presynaptic and/or postsynaptic response include, but are not limited to, for example, at least one of: increased of spontaneous transmitter release; modification of noise kinetics; increase in a postsynaptic response (e.g., absent an increase in presynaptic ICa⁺⁺ amplitude); decrease in synaptic vesicle density and/or number at active zones; increase in the number of clathrin-coated vesicles, and/or large endosome like vesicles near junctional sites; increase in ATP synthesis (e.g., at the presynaptic and postsynaptic terminals); or enhanced recovery of postsynaptic spike generation.

In particular aspects, the electrokinetically-altered ionic aqueous solutions optimize synaptic transmission without producing over abnormal over-release effects.

In particular aspects, the effect of artificial seawater (ASW) based on RNS60, a physically modified isotonic saline that has been electrokinetically altered to include charge-stabilized oxygen containing nanobubbles, has been shown to enhance and/or optimize neurotransmission.

### BRIEF DESCRIPTION OF THE DRAWINGS

FIGS. 1A through 1H show the effects of RNS60, PNS60, and NS on NMDA and AMPA-dependent calcium influx in cultured mouse hippocampal neurons.

FIGS. 2A through 2K show the effects of RNS60 in the expression of plasticity-associated proteins in mouse hippocampal neurons.

FIGS. 3A through 3Dviii show the effects of RNS60 on the expression of plasticity-associated genes in cultured mouse hippocampal neurons.

FIGS. 4A through 4D show the role of PI3K pathway in RNS60-mediated upregulation of plasticity-associated genes in mouse hippocampal neurons.

FIGS. 5A through 5D show that activation of PI3K regulates both NMDA- and AMPA-sensitive calcium influx in RNS60-treated mouse hippocampal neurons.

FIGS. 6A through 6J show the effect of RNS60 on the expression of plasticity-associated molecules in vivo in the hippocampus of 5XFAD transgenic animals.

FIGS. 7A through 7K show the effect of RNS60, NS, PNS60, and RNS10.3 on the number, size, and maturation of dendritic spines in hippocampal neurons.

FIGS. 8A through 8F show that RNS60 stimulates the length, and collaterals of primary axon in cultured hippocampal neurons.

FIGS. 9A, 9B(i)-9B(iii) and 9C-9E show activation of PI3K regulates morphological plasticity in RNS60-treated mouse hippocampal neurons.

FIG. 10 shows, according to particular exemplary aspects, an example of increased evoked transmitter release in a hypoxic synapse following electrical stimulation of the presynaptic terminal.

FIGS. 11A-11E show, according to particular exemplary aspects, high-frequency stimulation in Control and RNS60 ASW.

FIGS. 12A-12C show, according to particular exemplary aspects, synaptic noise recorded in Control ASW and RNS60 ASW.

FIGS. 13A-13E show, according to particular exemplary aspects, a voltage clamp study indicating that RNS60 increases transmitter release without modifying calcium current or its relationship with transmitter release.

FIGS. 14A-14F show, according to particular exemplary aspects, direct determination of increased ATP synthesis at the presynaptic and postsynaptic terminals using Luciferin/Luciferase light emission.

FIG. 15 shows, according to particular exemplary aspects, reduction of spontaneous synaptic release following oligomycin administration. Plot of noise amplitude as a function of frequency (note double log coordinates). Red is Control ASW, green is 7 min after addition of oligomycin and blue is 22 min after oligomycin administration and 12 min after changing superfusion to RNS60 ASW. Black is extracellular recording.

FIGS. 16A-16C show, according to particular exemplary aspects, electronmicrographs of a synaptic junction following RNS60 ASW superfusion.

FIGS. 17A and 17B show, according to particular exemplary aspects, statistical determination of synaptic vesicle numbers in synapses superfused with RNS60 ASW. FIG. 8A shows a plot of the number of CCV as a function of size. FIG. 8B shows the number of large vesicles as a function of size.

FIGS. 18A-18C show, according to particular exemplary aspects, the ultrastructure of squid giant synapse active zones following oligomycin injection.

FIGS. 19A-19C show, according to particular exemplary aspects, the effect of RNS60 and olygomycin on synaptic vesicle numbers.

### DETAILED DESCRIPTION OF THE INVENTION

### Upregulating/Enhancing Hippocampal Plasticity and Hippocampus-Dependent Learning and Memory

Certain embodiments disclosed herein relate to providing compositions and methods for upregulating hippocampal plasticity and hippocampus-dependent learning and memory, comprising administering, to a subject (e.g., a mammal or human) in need thereof, a therapeutic composition comprising an electrokinetically-altered, gas-enriched (e.g., oxygen enriched) aqueous fluid.

Particular aspects provide a method for enhancing hippocampal plasticity and hippocampus-dependent learning and memory, comprising administering to a subject in need thereof a therapeutically effective amount of an electrokinetically altered aqueous fluid comprising an ionic aqueous solution of charge-stabilized oxygen-containing nanostructures having an average diameter of less than about 100 nanometers and stably configured in the ionic aqueous fluid in an amount sufficient for enhancing hippocampal plasticity and hippocampus-dependent learning and memory to provide a method for enhancing hippocampal plasticity and hippocampus-dependent learning and memory in the subject.

Increased calcium influx through ionotropic glutamate receptors and the upregulation of plasticity-associated molecules in hippocampal neurons are two important events in the process of hippocampus-dependent spatial learning and memory. Here we have undertaken an innovative approach to upregulate hippocampal plasticity. Applicants' RNS60 fluid, for example, is an isotonic saline solution generated by subjecting normal saline to a patented type of Taylor-Couette-Poiseuille (TCP) flow under elevated oxygen pressure (see, e.g., Applicants' issued U.S. Pat. Nos. 7,832,920, 7,919,534, 8,410,182, 8,445,546, 8,449,172, and 8,470,893, all incorporated herein by reference in their respective entireties).

RNS60, but neither NS (normal saline) nor PNS60 (saline containing excess oxygen without TCP modification) stimulates the NMDA- and AMPA-sensitive calcium influx in cultured hippocampal neurons. Using mRNA-based targeted gene array, real-time PCR, and immunoblot and immunofluorescence analysis, we further demonstrate that RNS60 stimulates the upregulation of many plasticity-associated proteins in cultured hippocampal neurons. Finally, RNS60 treatment increased plasticity-associated proteins and calcium influx in the hippocampus of 5XFAD transgenic mouse model of Alzheimer's disease (AD). These results describe a novel property of RNS60 in stimulating hippocampal plasticity, which may be helpful in treating AD and other dementias.

According to particular aspects, the disclosed electrokinetically-altered fluids (e.g., RNS60) control or modulate (e.g., increase or enhance) the synaptic plasticity of hippocampal neurons by inducing calcium influx via NMDA- and AMPA-sensitive ionotropic glutamate receptors. RNS60, but neither NS nor PNS, stimulates the expression of NR2A, NR2B subunits NMDA and GluR1 subunit of AMPA receptors along with other plasticity-associated molecules including Arc, PSD95, and CREB.

It is believed that plasticity decreases in various conditions including, but not limited to, old age and in patients with AD. Therefore, exploring ways to boost plasticity generally, including in conditions of learning disorders and in AD or aging is an important area of research. Although there are other drugs and approaches for improving brain function, here we introduce a simple saline-based agent to augment plasticity. Upon subjecting normal saline to Taylor-Couette-Poiseuille (TCP) turbulence in the presence of elevated oxygen pressure, Revalesio Corporation (Tacoma, Wash.) has generated RNS60, which does not contain any active pharmaceutical ingredient (19, 20). Due to TCP turbulence, RNS60 contains charge-stabilized nanostructures consisting of, e.g., an oxygen nanobubble core surrounded by an electrical double-layer at the liquid/gas interface (19, 20).

Here we delineate the first evidence that ionic fluid or saline generated due to TCP turbulence is capable of improving plasticity in cultured hippocampal neurons and in vivo (e.g., in the hippocampus of 5XFAD transgenic mice).

Our conclusion is based on the following:

First, as shown in Example 7, we observed that RNS60 induced the number, size, and maturation of dendritic spines in cultured hippocampal neurons, indicating a beneficial role of RNS60 in regulating the synaptic efficacy of neurons;

Second, as shown in Example 7, RNS60 increased the axonal length and collaterals in neurons further corroborating the role of RNS60 in stimulating the morphological plasticity of neurons.

Third, as shown in working Example 3, RNS60 did not alter the calcium dependent excitability of hippocampal neurons, but rather stimulated inbound calcium currents in these neurons through ionotropic glutamate receptor. This indicates that RNS60 modulates plasticity-related activities.

Fourth, as shown in working Example 4, RNS60 induced the expression of a broad spectrum of plasticity-associated molecules in hippocampal neurons.

Fifth, as shown in working Example 4, RNS60 augmented the levels of several genes, proteins of which stimulate signaling pathways (adenylate cyclase, CAM kinase II and Akt) for the activation of CREB, the master regulator of plasticity.

Sixth, as shown in working Example 4, proteins encoded by several genes such as Gria2, Ppp1ca, Ppp2ca, and Ppp3ca are known to support long-term depression (35). It is interesting to see that RNS60 down-regulated the expression of Gria2, Ppp1ca, Ppp2ca, and Ppp3ca in hippocampal neurons.

Seventh, as shown in working Example 6, RNS60 treatment increased the expression of plasticity-associated molecules and augmented calcium influx in vivo in the hippocampus of 5XFAD transgenic mice. These results indicates that RNS60 provides as a therapeutic agent in boosting plasticity in patients in need thereof, including subject with learning and/or memory disorders, and including subjects with neuronal injury, and those with AD and other dementias.

A growing body of evidence suggests that the excessive activation of glutamate-operated NMDA receptors in postsynaptic neurons is the primary factor of progressive neuronal loss in AD (28). Different noncompetitive and uncompetitive NMDA receptor blockers are being used for the treatment of AD (36). However prolonged use of these drugs eventually destroys the normal excitability of these receptors, which is essential for the viability of these neurons. Moreover, these specific inhibitors of NMDA receptors generate a wide range of side effects including chest pain, nausea, increased heart rate, breathing trouble, lowered urination, and different digestive disorders because of their poor metabolic clearance among older populations (37, 38). In contrast, RNS60 for example, produces almost no side effects as chemically it is identical to isotonic saline with additional oxygen.

As presently disclosed in working Examples 2 through 6, RNS60 treatment generated high amplitude NMDA-dependent calcium oscillations both in cell culture and in vivo experiments. Since high amplitude calcium wave corresponds to the excitability of ionotropic receptors, if follows that RNS60 does not alter the normal excitability of NMDA receptors. Moreover, RNS60 induced the expression many growth supportive molecules including CREB, BDNF and NTRs, which are required for the survival of neurons; synaptic proteins including PSD95, ADAM-10, and Synpo, which are required for the maintenance of synaptic structure; receptor proteins including NR2A, GluR1, and NR2B, which are needed for calcium excitability of the postsynaptic neurons; and IEGs such as c-FOS, Arc, Homer 1, and Zif-268 essential for neuroplasticity, leading to memory consolidation (39-41).

Signaling mechanisms leading to plasticity are becoming clear. It has been found that master regulator cAMP response element-binding (CREB) plays an important role in plasticity and promoters of different plasticity-associated genes harbor multiple cAMP response elements (CRE) (42-45). Applicants have demonstrated that RNS60 induces the activation of CREB in microglial cells via type IA phosphatidylinositol 3-kinase (PI3K) in microglial cells. PI3K is a key signaling molecule implicated in the regulation of a broad array of biological responses including cell survival (34). For class IA PI3K, the p85 regulatory subunit acts as an interface by interacting with the IRS-1 through its SH2 domain and thus recruits the p110 catalytic subunit (p110α/β) to the cell membrane, which in turn activates downstream signaling molecules like Akt/protein kinase B and p70 ribosomal S6 kinase (34). On the other hand, for class IB PI3K, p110γ is activated by the engagement of G-protein coupled receptors. The p110γ then catalyzes the reaction to release phosphatidylinositol (3,4,5)-triphosphate as the second messenger, using phosphatidylinositol (4,5)-bisphosphate as the substrate, and activates downstream signaling molecules (33).

Herein we demonstrate, in working Example 5, that RNS60 induces the activation of both the subunits of type IA PI-3K (p110α and p110β) without modulating type IB PI-3K p110γ in primary hippocampal neurons, indicating the specific activation of type IA p110α/β PI3K in neurons. Furthermore, abrogation of RNS60-mediated upregulation of NR2A and GluR1 and stimulation of calcium influx in hippocampal neurons by inhibitors of PI3K indicates that RNS60 increases NMDA- and AMPA-sensitive calcium current via PI3K.

According to particular aspects, applicants herein demonstrate, for the first time, that RNS60 treatment upregulates plasticity-associated molecules and calcium influx in cultured hippocampal neurons and in vivo (e.g., in the hippocampus of 5XFAD mice). These results demonstrate and confirm a new hippocampal neuron plasticity boosting property of applicants' fluids (e.g., RNS60) and provide a new use for applicants' modified saline for stimulating synaptic plasticity in all types of subjects as disclosed herein.

### Optimizing Neuronal Synaptic Transmission

According to particular exemplary aspects, RNS60, a physically modified saline containing charge-stabilized oxygen-containing nanostructures (e.g., charge-stabilized oxygen-containing nanobubbles), has significant function-optimizing properties for optimizing neuronal synaptic transmission.

According to particular aspects, RNS60 represents a class of bioactive agents relating to the physical structure of water and an increased oxygen caring ability (in the form of charge-stabilized oxygen-containing nanostructures, e.g., charge-stabilized oxygen-containing nanobubbles having an average diameter less than 100 nm), with no added chemical molecules and yet has proven cytoprotective and anti-inflammatory effects in different models of neurodegeneration through direct effects on glial cells as well as modulation of T cell subsets (Khasnavis S. 2012; Mondal, S, 2012). Without being bound by mechanism, and together with the results described herein, this suggests that RNS60 exerts pleiotropic effects that are not based on interaction with a specific receptor, but rather that RNS60 is a facilitator of physiological function that require a different appellative. Functionally, as shown herein, RNS60 is able to optimize synaptic transmission without affecting normal function, and without any deleterious side effects (as has been demonstrated in previous studies in other systems including human use where no deleterious effects have been seen).

Preferred embodiments. Particular aspects provide a method for optimizing neurotransmission, comprising contacting neurons with, or administrating to a subject having neurons, an electrokinetically-altered ionic aqueous solution comprising charge-stabilized oxygen-containing nanostructures having an average diameter of less than 100 nm in an amount and for a time period sufficient for modulating at least one presynaptic and/or postsynaptic response, wherein a method for optimizing neuronal synaptic transmission is afforded. In certain aspects, modulating at least one presynaptic and/or postsynaptic response comprises an increase of spontaneous transmitter release. In certain aspects, modulating at least one presynaptic and/or postsynaptic response comprises a modification of noise kinetics. In certain aspects, modulating at least one presynaptic and/or postsynaptic response comprises an increase in a postsynaptic response (e.g., without an increase in presynaptic ICa⁺⁺ amplitude). In certain aspects, modulating at least one presynaptic and/or postsynaptic response comprises a decrease in synaptic vesicle density and/or number at active zones, and may further comprise an increase in the number of clathrin-coated vesicles, and large endosome like vesicles in the vicinity of the junctional sites. In certain aspects, modulating at least one presynaptic and/or postsynaptic response comprises a marked increase in ATP synthesis leading to synaptic transmission optimization. In certain aspects, modulating at least one presynaptic and/or postsynaptic response comprises an enhanced or more vigorous recovery of postsynaptic spike generation. In certain aspects, modulating at least one presynaptic and/or postsynaptic response comprises increased ATP synthesis at the presynaptic and postsynaptic terminals. In particular embodiments the charge-stabilized oxygen-containing nanostructures having an average diameter of less than 100 nm comprise charge-stabilized oxygen-containing nanobubbles having an average diameter of less than 100 nm.

Additional aspect provide a method for optimizing neurotransmission, comprising contacting neurons with, or administrating to a subject having neurons, an electrokinetically-altered ionic aqueous solution comprising charge-stabilized oxygen-containing nanostructures having an average diameter of less than 100 nm in an amount and for a time period sufficient for enhancing intracellular oxygen delivery or utilization, wherein a method for optimizing neuronal synaptic transmission is afforded. In certain aspects, optimizing neuronal synaptic transmission comprises an increase of spontaneous transmitter release. In certain aspects, optimizing neuronal synaptic transmission comprises a modification of noise kinetics. In certain aspects, optimizing neuronal synaptic transmission comprises an increase in a postsynaptic response (e.g., without an increase in presynaptic ICa⁺⁺ amplitude). In certain aspects, optimizing neuronal synaptic transmission comprises a decrease in synaptic vesicle density and/or number at active zones, and may further comprise an increase in the number of clathrin-coated vesicles, and large endosome like vesicles in the vicinity of the junctional sites. In certain aspects, optimizing neuronal synaptic transmission comprises a marked increase in ATP synthesis. In certain aspects, optimizing neuronal synaptic transmission comprises an enhanced or more vigorous recovery of postsynaptic spike generation. In certain aspects, optzing neuronal synaptic transmission comprises increased ATP synthesis at the presynaptic and postsynaptic terminals. In particular embodiments the charge-stabilized oxygen-containing nanostructures having an average diameter of less than 100 nm comprise charge-stabilized oxygen-containing nanobubbles having an average diameter of less than 100 nm.

Further aspect provide a method for enhancing intracellular oxygen delivery or utilization, comprising contacting cells with, or administrating to a subject having cells, an electrokinetically-altered ionic aqueous solution comprising charge-stabilized oxygen-containing nanostructures having an average diameter of less than 100 nm in an amount and for a time period sufficient for enhancing intracellular oxygen delivery or utilization in the cells. In particular aspects, the cells are nerve cells (e.g., mammalian, human or other; any organism or animal comprising neurons and neuronal transmission). In particular aspects, enhancing intracellular oxygen delivery or utilization provides for optimizing neuronal synaptic transmission. In particular aspects, optimizing neuronal synaptic transmission comprises an increase of spontaneous transmitter release. In particular aspects, optimizing neuronal synaptic transmission comprises a modification of noise kinetics. In particular aspects, optimizing neuronal synaptic transmission comprises an increase in a postsynaptic response (e.g., without an increase in presynaptic ICa⁺⁺ amplitude). In particular aspects, optimizing neuronal synaptic transmission comprises a decrease in synaptic vesicle density and/or number at active zones. Particular aspects may further comprise an increase in the number of clathrin-coated vesicles, and large endosome like vesicles in the vicinity of the junctional sites. In particular aspects, optimizing neuronal synaptic transmission comprises a marked increase in ATP synthesis. In particular aspects, optimizing neuronal synaptic transmission comprises an enhanced or more vigorous recovery of postsynaptic spike generation. In particular aspects, optimizing neuronal synaptic transmission comprises increased ATP synthesis at the presynaptic and postsynaptic terminals. In particular embodiments the charge-stabilized oxygen-containing nanostructures having an average diameter of less than 100 nm comprise charge-stabilized oxygen-containing nanobubbles having an average diameter of less than 100 nm.

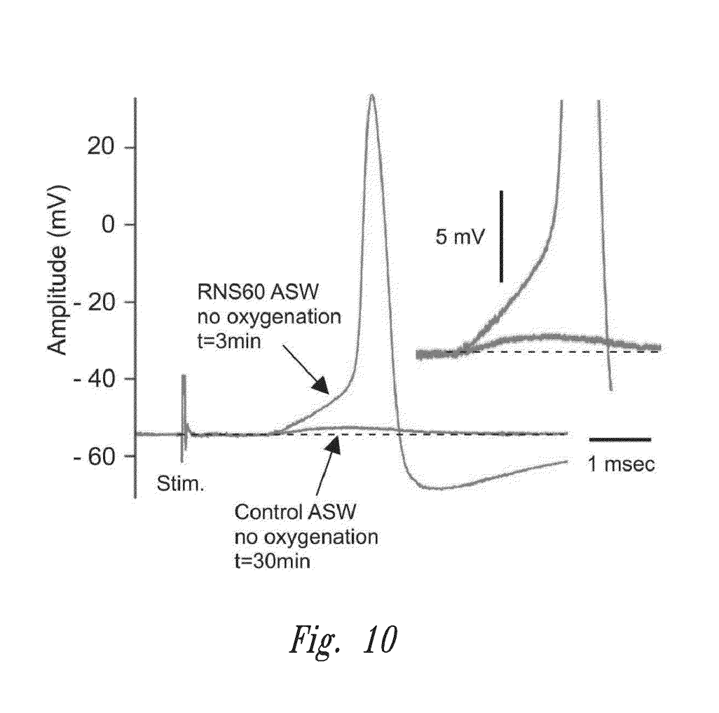

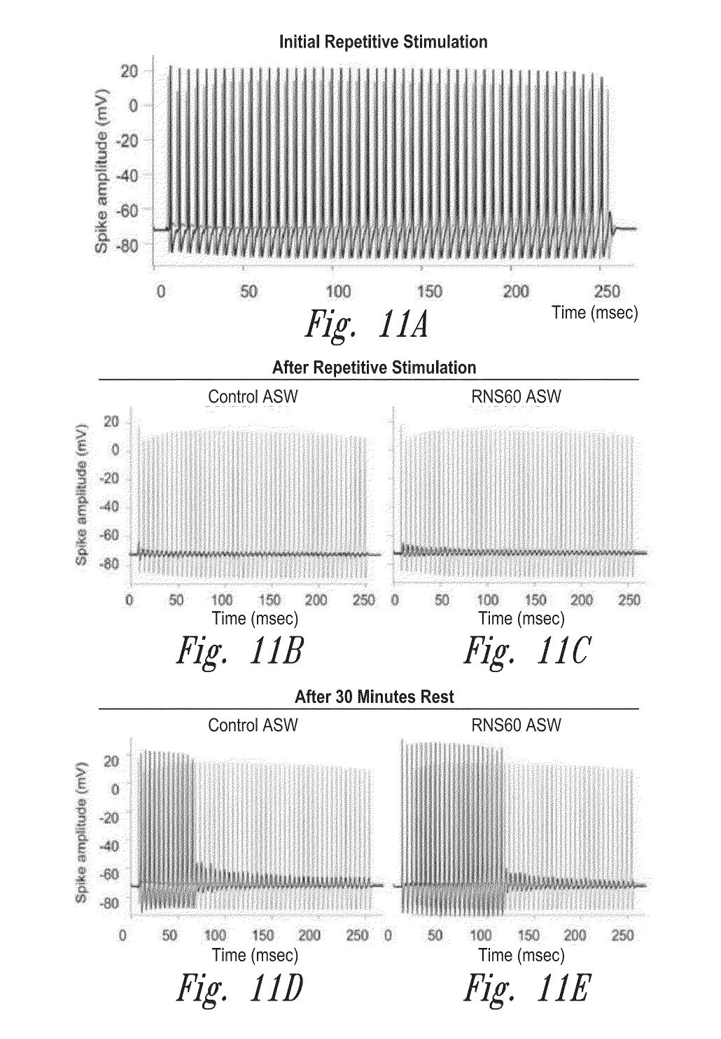

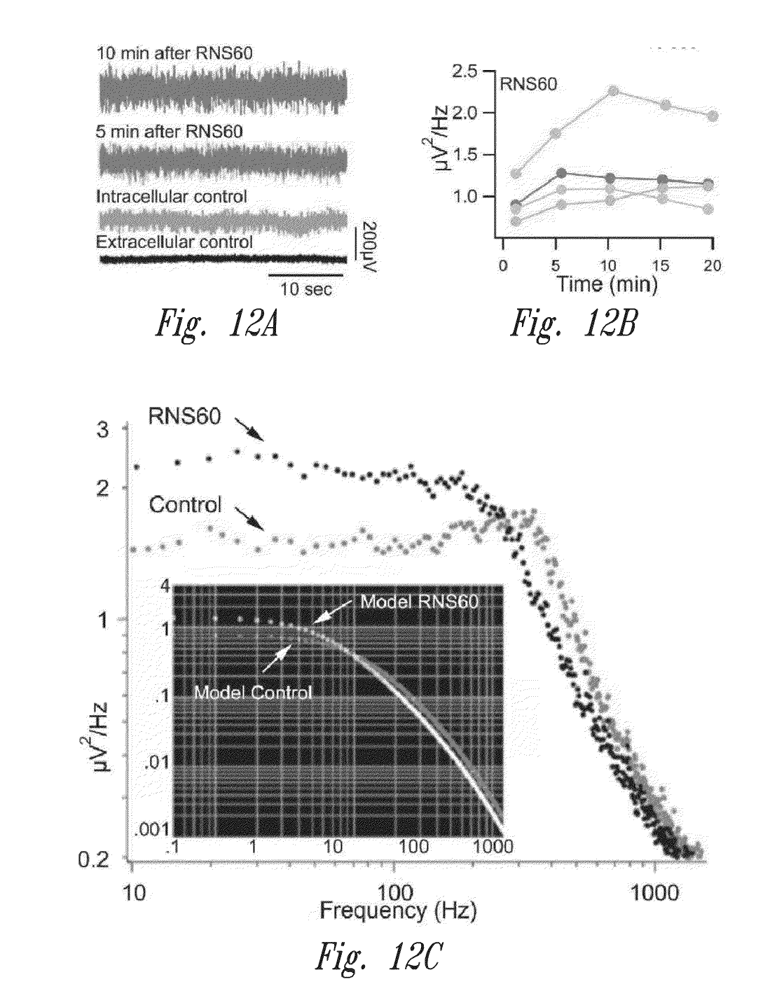

Consistent with the above, the disclosed results concerning single spike synaptic transmission (FIG. 10; working Example 9), as well as the response to repetitive presynaptic terminal activation (FIG. 11; working Example 10) indicate that the ability of RNS60 to maintain and optimize synaptic transmission within normal parameters is not accompanied by abnormal responses indicating the absence of overdose or side effects. This conclusion is also supported by the increase in spontaneous release that reaches a maximum level following a single superfusion of RNS60 that is maintained for a period of 30 minutes and decays slowly after superfusion with Control ASW (FIG. 12; working Example 11). Similar results were found with the increase in spontaneous transmitter release (FIG. 12; working Example 11).

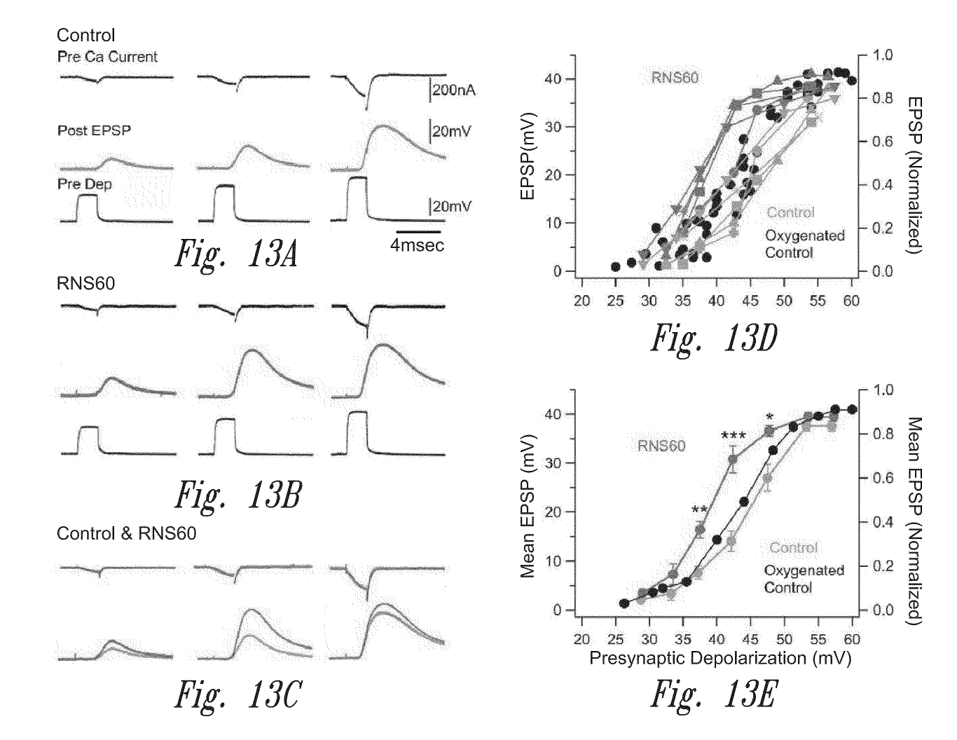

With respect to the mechanism of action of RNS60 in the claimed methods, the possibility that it could be modifying channel kinetics, and in particular calcium currents, was rendered unlikely by the voltage clamp results which indicate that synaptic optimization is not correlated with any change in the time course or amplitude of the inward calcium current responsible for the transmitter release (FIG. 13; working Example 12).

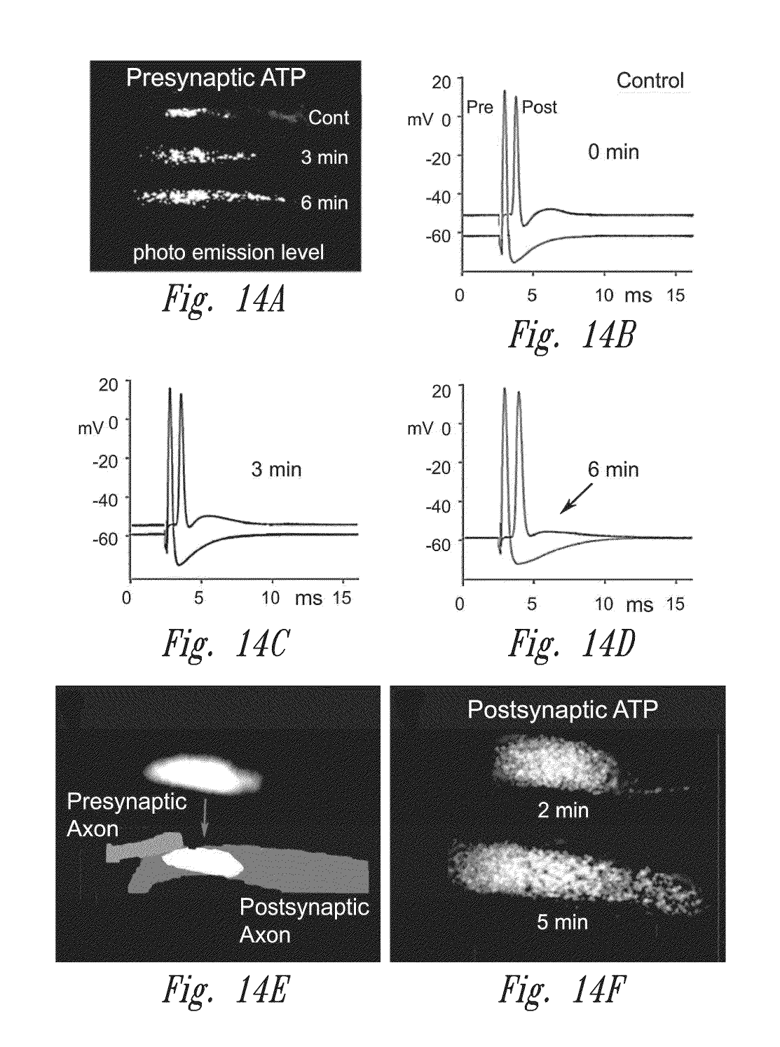

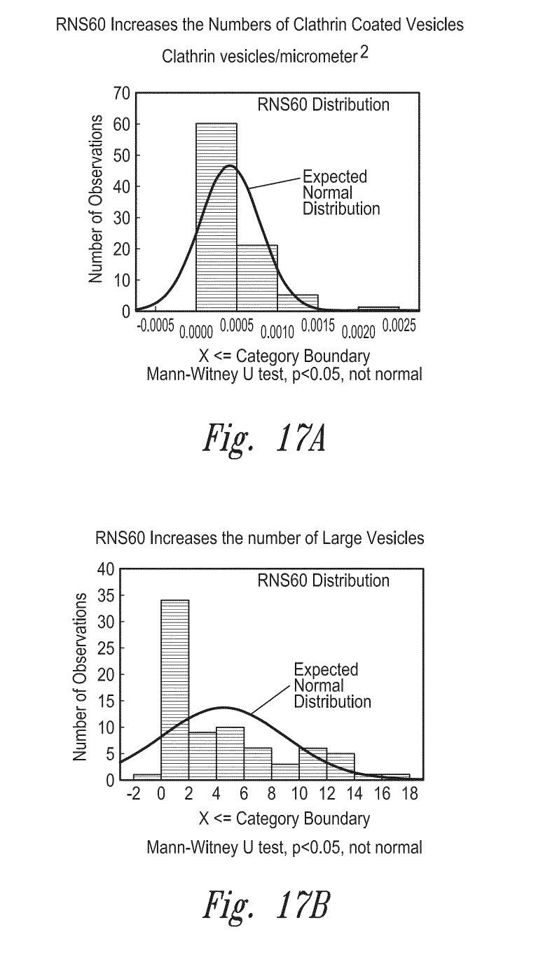

Without being bound by mechanism, RNS60 likely changes available energy level, via ATP increase and that such event is accompanied by an increase in synaptic transmission effectiveness (FIG. 14; working Example 13). An additional unexpected finding was that of the noise frequency change in the presence of RNS60 (FIG. 12C; working Example 11). The fact that at the level of spontaneous release there is a clear change in the noise profile, seen as a reduction of high frequency noise and an increase of low frequency noise (FIG. 12C; working Example 11), appears to correlate with the change in the synaptic vesicle size distribution (FIG. 17; working Example 15). Without being bound by mechanism, the transmitter delivery kinetics may be different between normal vesicular profiles and that of the larger endosome related vesicles. The latter may have a slower release kinetics that may explain the change in noise frequency towards lower frequency with an accompanying noise level amplitude increase.

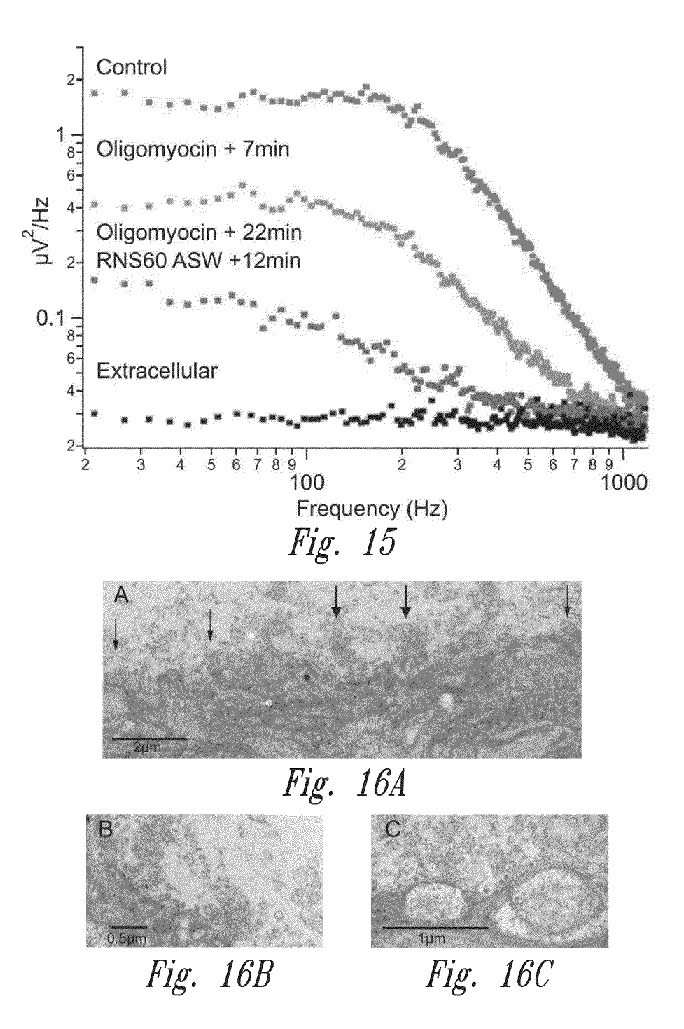

From a morphological perspective, it is known that increased expression of the brain vesicular monoamine transporter VMAT2 regulates vesicle phenotype and quantal size (Pothos F N et. al, 2000). As shown in RNS60 superfused terminals (FIG. 16 A; working Example 15), large vesicles with different shapes and sizes are observed in the analyzed terminals. These structures are never observed in the current control synapses (FIG. 15B; working Example 14) or in terminals studied in former experiments (Heuser, J. E. & Reese, T. S., 1973).

Neurotransmitter release requires a well-known set of steps concerning synaptic vesicle exo- and endocytosis (Heuser, J. E. and Reese T. S., 1973). It has been shown in previous work that dinamin/synaptophysin complex disruption results in a decrease of transmitter release, resulting from a depletion of synaptic vesicle recycling (Daly C., et al. 2000). It has also been observed that, under these conditions, the number of CCVs actually increased suggesting the existence of another vesicle endocytosis mechanism with a faster time course than the classical clathrin pathway (Daly et al. 2000). This finding was further corroborated by the injection of Rabfilin 3A and/or one of its fragments which affect the distribution of membranes of the endocytotic pathway in the squid presynaptic terminal in a multifunctional fashion (Burns M E et al., 1998). This is consistent with previous observations following different domains manipulation of the synaptic vesicle protein synaptotagmin (Mikoshiba K. et al. 1995; Fukuda M. et al, 1995).

Although the synaptic hyperactivity demonstrated herein after RNS60 administration is accompanied by a significant decrease in synaptic vesicles numbers at the active zone, the presynaptic terminal area at the vicinity of the active zone showed a large number of CCVs, the amount of membrane retrieved by CCVs may not be sufficient to maintain of the large amount of transmitter release observed during the augmented synaptic release described here. However, the large number of endosomal vesicles (up to 300 nm in diameter) that were imaged in the immediate vicinity of the active zone could be part of the enhanced synaptic transmitter released observed under these conditions. This was supported by the presence of such “larger vesicles” at the active zone intermingled with usual synaptic vesicle profiles (FIG. 17; working Example 15). Since such vesicles appear throughout the active zone vicinity, it suggests that the endocytotic mechanism responsible for their presence may be independent of the clathrin or caveolin pathway (reviewed by Mayor S. and Pagano R. E. 2007).

The fact that both spontaneous release levels as well as the amplitude of the evoked synaptic potentials are increased significantly indicates that while the probability of release of regular sized vesicles may be slightly decreased, the release of the larger vesicular component may actually be increased. Such a change in the distribution of vesicular size, favoring the larger endosomal vesicular profiles over the smaller clathrin related vesicles, confirms a similar morphological analysis of vesicular size distribution following high level synaptic activation (Hayashi M et al 2008, as reviewed by Saheki, Y and De Camili P. 2012).

This change in vesicular size distribution may provide a possible explanation for the fact that the nature of the spontaneous synaptic noise was modified after RNS 60 administration, as shown in FIG. 12 and as discussed in the description of synaptic noise and its relation to time course of synaptic miniature potentials and vesicular size (working Example 11).

Mitochondria are energy-supplier organelles, strikingly abundant in chemical synapses (Palay, S I 1956, Talbot J. D. et al., 2003). In squid the presynaptic terminal mitochondria lies in close juxtaposition to presynaptic calcium channels (Pivovarova N B. et al., 1999). Energy supply to neurons in the form of oxygen and glucose and its final product—mitochondrial generated ATP, is largely used for reversing the ion influxes underlying synaptic and action potentials (Attwell D. and Laughlin S B. 2001). Here Applicants tested whether inhibition of mitochondria ATP with oligomycin, modified the effect of RNS60 on synaptic transmission.

Mitochondria may be blocked with drugs that do not alter mitochondrial membrane potential (Ψₘ), such as oligomycin or with depolarizing Ψₘ inhibitors. Ru360, an inhibitor of the mitochondrial uniporter was not used because in some terminals Ru360 appears to inhibit Ca²⁺ influx across the plasma membrane (David G. 1999). The use of CCCP or Antimycin A1 was also avoided as these are also Ψₘ depolarizing agents, and because both of them can potentially affect transmitter release from presynaptic terminals, since these agents block mitochondrial calcium uptake.

Concomitant application of RNS60 and the complex V mitochondrial blocker (olygomicin) failed to induce increments in spontaneous release as determined by synaptic noise power spectrum analysis (FIG. 15; working Example 14). These experiments suggest that RNS60 mechanism of action is dependent on mitochondrial ATP production, potentially by providing, or facilitating provision of oxygen in a more efficient manner.

Concerning the mechanism of action of RNS60, it may be significant that a block of mitochondrial ATP synthesis results in an inactivation of the RNS60 effect on synaptic transmission. These findings further indicate that the reduction of ATP synthesis is accompanied by a lack of response of synaptic release mechanism by RNS60. These findings indicate that RNS60 likely does not operate directly on the vesicular release mechanism, but rather indirectly via an increased synthesis of ATP by the mitochondrial system. This has been shown to have a significant effect on both the availability of vesicular organelles and on their movement on to the active zone at the presynaptic compartment in the synaptic junction region (Ivanikov M V. et al. 2010).

According to particular aspects, therefore, which respect to optimizing neurotransmission, RNS60 is an ATP synthesis optimizer via facilitation of oxygen transport into the mitochondrial system, with minimal increase in intracellular free radical level.

### Electrokinetically-Generated Fluids:

“Electrokinetically generated fluid,” as used herein, refers to Applicants' inventive electrokinetically-generated fluids generated, for purposes of the working Examples herein, by the exemplary Mixing Device described in detail in Applicants' issued patents (see, e.g., Applicants' issued U.S. Pat. Nos. 7,832,920, 7,919,534, 8,410,182, 8,445,546, 8,449,172, and 8,470,893, all incorporated herein by reference in their respective entireties). The electrokinetic fluids, as demonstrated by the data disclosed and presented herein, represent novel and fundamentally distinct fluids relative to prior art non-electrokinetic fluids, including relative to prior art oxygenated non-electrokinetic fluids (e.g., pressure pot oxygenated fluids and the like). As disclosed in various aspects herein, the electrokinetically-generated fluids have unique and novel physical and biological properties including, but not limited to the following:

In particular aspects, the electrokinetically altered aqueous fluid comprise an ionic aqueous solution of charge-stabilized oxygen-containing nanostructures substantially having an average diameter of less than about 100 nanometers and stably configured in the ionic aqueous fluid in an amount sufficient to provide, upon contact of a living cell by the fluid, modulation of at least one of cellular membrane potential and cellular membrane conductivity.

In preferred aspects, RNS60 is a physically modified normal saline (0.9%) solution generated by using a rotor/stator device, which incorporates controlled turbulence and Taylor-Couette-Poiseuille (TCP) flow under high oxygen pressure (see Applicants U.S. Pat. Nos. 7,832,920, 7,919,534, 8,410,182, 8,445,546, 8,449,172, and 8,470,893, all incorporated herein by reference in their entireties for their teachings encompassing Applicants' device, methods for making the fluids, and the fluids per se).

In particular aspects, electrokinetically-generated fluids refers to fluids generated in the presence of hydrodynamically-induced, localized (e.g., non-uniform with respect to the overall fluid volume) electrokinetic effects (e.g., voltage/current pulses), such as device feature-localized effects as described herein. In particular aspects said hydrodynamically-induced, localized electrokinetic effects are in combination with surface-related double layer and/or streaming current effects as disclosed and discussed herein.

In particular aspects the administered inventive electrokinetically-altered fluids comprise charge-stabilized oxygen-containing nanostructures in an amount sufficient to provide modulation of at least one of cellular membrane potential and cellular membrane conductivity. In certain embodiments, the electrokinetically-altered fluids are superoxygenated (e.g., RNS-20, RNS-40 and RNS-60, comprising 20 ppm, 40 ppm and 60 ppm dissolved oxygen, respectively, in standard saline). In particular embodiments, the electrokinetically-altered fluids are not-superoxygenated (e.g., RNS-10 or Solas, comprising 10 ppm (e.g., approx. ambient levels of dissolved oxygen in standard saline)). In certain aspects, the salinity, sterility, pH, etc., of the inventive electrokinetically-altered fluids is established at the time of electrokinetic production of the fluid, and the sterile fluids are administered by an appropriate route. Alternatively, at least one of the salinity, sterility, pH, etc., of the fluids is appropriately adjusted (e.g., using sterile saline or appropriate diluents) to be physiologically compatible with the route of administration prior to administration of the fluid. Preferably, and diluents and/or saline solutions and/or buffer compositions used to adjust at least one of the salinity, sterility, pH, etc., of the fluids are also electrokinetic fluids, or are otherwise compatible.

In particular aspects, the inventive electrokinetically-altered fluids comprise saline (e.g., one or more dissolved salt(s); e.g., alkali metal based salts (Li+, Na+, K+, Rb+, Cs+, etc.), alkaline earth based salts (e.g., Mg++, Ca++), etc., or transition metal-based positive ions (e.g., Cr, Fe, Co, Ni, Cu, Zn, etc.), in each case along with any suitable anion components, including, but not limited to F-, Cl-, Br-, I-, PO4-, SO4-, and nitrogen-based anions. Particular aspects comprise mixed salt based electrokinetic fluids (e.g., Na+, K+, Ca++, Mg++, transition metal ion(s), etc.) in various combinations and concentrations, and optionally with mixtures of couterions. In particular aspects, the inventive electrokinetically-altered fluids comprise standard saline (e.g., approx. 0.9% NaCl, or about 0.15 M NaCl). In particular aspects, the inventive electrokinetically-altered fluids comprise saline at a concentration of at least 0.0002 M, at least 0.0003 M, at least 0.001 M, at least 0.005 M, at least 0.01 M, at least 0.015 M, at least 0.1 M, at least 0.15 M, or at least 0.2 M. In particular aspects, the conductivity of the inventive electrokinetically-altered fluids is at least 10 μS/cm, at least 40 μS/cm, at least 80 μS/cm, at least 100 μS/cm, at least 150 μS/cm, at least 200 μS/cm, at least 300 μS/cm, or at least 500 μS/cm, at least 1 mS/cm, at least 5, mS/cm, 10 mS/cm, at least 40 mS/cm, at least 80 mS/cm, at least 100 mS/cm, at least 150 mS/cm, at least 200 mS/cm, at least 300 mS/cm, or at least 500 mS/cm. In particular aspects, any salt may be used in preparing the inventive electrokinetically-altered fluids, provided that they allow for formation of biologically active salt-stabilized nanostructures (e.g., salt-stabilized oxygen-containing nanostructures) as disclosed herein.

According to particular aspects, the biological effects of the inventive fluid compositions comprising charge-stabilized gas-containing nanostructures can be modulated (e.g., increased, decreased, tuned, etc.) by altering the ionic components of the fluids, and/or by altering the gas component of the fluid.

According to particular aspects, the biological effects of the inventive fluid compositions comprising charge-stabilized gas-containing nanostructures can be modulated (e.g., increased, decreased, tuned, etc.) by altering the gas component of the fluid. In preferred aspects, oxygen is used in preparing the inventive electrokinetic fluids. In additional aspects mixtures of oxygen along with at least one other gas selected from Nitrogen, Oxygen, Argon, Carbon dioxide, Neon, Helium, krypton, hydrogen and Xenon. As described above, the ions may also be varied, including along with varying the gas constitutent(s).

Given the teachings and assay systems disclosed herein (e.g., cell-based cytokine assays, patch-clamp assays, etc.) one of skill in the art will readily be able to select appropriate salts and concentrations thereof to achieve the biological activities disclosed herein.

TABLE 1
Exemplary cations and anions.

| | | |
|---|---|---|
| Name | Formula | Other name(s) |

Common Cations:

| | | |
|---|---|---|
| Aluminum | Al⁺³ | |
| Ammonium | NH₄⁺ | |
| Barium | Ba⁺² | |
| Calcium | Ca⁺² | |
| Chromium (II) | Cr⁺² | Chromous |
| Chromium (III) | Cr⁺³ | Chromic |
| Copper (I) | Cu⁺ | Cuprous |
| Copper (II) | Cu⁺² | Cupric |
| Iron (II) | Fe⁺² | Ferrous |
| Iron (III) | Fe⁺³ | Ferric |
| Hydrogen | H⁺ | |
| Hydronium | H₃O⁺ | |
| Lead (II) | Pb⁺² | |
| Lithium | Li⁺ | |
| Magnesium | Mg⁺² | |
| Manganese (II) | Mn⁺² | Manganous |
| Manganese (III) | Mn⁺³ | Manganic |
| Mercury (I) | Hg₂⁺² | Mercurous |
| Mercury (II) | Hg⁺² | Mercuric |
| Nitronium | NO₂⁺ | |
| Potassium | K⁺ | |
| Silver | Ag⁺ | |
| Sodium | Na⁺ | |
| Strontium | Sr⁺² | |
| Tin (II) | Sn⁺² | Stannous |
| Tin (IV) | Sn⁺⁴ | Stannic |
| Zinc | Zn⁺² | |

Common Anions:

| | | | |
|---|---|---|---|
| Simple ions: | | | |
| Hydride | H⁻ | Oxide | O²⁻ |
| Fluoride | F⁻ | Sulfide | S²⁻ |
| Chloride | Cl⁻ | Nitride | N³⁻ |
| Bromide | Br⁻ | | |
| Iodide | I⁻ | | |
| Oxoanions: | | | |
| Arsenate | AsO₄³⁻ | Phosphate | PO₄³⁻ |
| Arsenite | AsO₃³⁻ | Hydrogen phosphate | HPO₄²⁻ |
| | | Dihydrogen | H₂PO₄⁻ |
| | | phosphate | |
| Sulfate | SO₄²⁻ | Nitrate | NO₃⁻ |
| Hydrogen sulfate | HSO₄⁻ | Nitrite | NO₂⁻ |
| Thiosulfate | S₂O₃²⁻ | | |
| Sulfite | SO₃²⁻ | | |
| Perchlorate | ClO₄⁻ | Iodate | IO₃⁻ |
| Chlorate | ClO₃⁻ | Bromate | BrO₃⁻ |
| Chlorite | ClO₂⁻ | | |
| Hypochlorite | OCl⁻ | Hypobromite | OBr⁻ |
| Carbonate | CO₃²⁻ | Chromate | CrO₄²⁻ |
| Hydrogen carbonate | HCO₃⁻ | Dichromate | Cr₂O₇²⁻ |
| or Bicarbonate | | | |
| Anions from Organic Acids: | | | |
| Acetate | CH₃COO⁻ | formate | HCOO⁻ |
| Others: | | | |
| Cyanide | CN⁻ | Amide | NH₂⁻ |
| Cyanate | OCN⁻ | Peroxide | O₂²⁻ |
| Thiocyanate | SCN⁻ | Oxalate | C₂O₄²⁻ |
| Hydroxide | OH⁻ | Permanganate | MnO₄⁻ |

TABLE 2
Exemplary cations and anions.

| | | |
|---|---|---|
| Formula | Charge | Name |

Monoatomic Cations

| | | |
|---|---|---|
| H⁺ | 1+ | hydrogen ion |
| Li⁺ | 1+ | lithium ion |
| Na⁺ | 1+ | sodium ion |
| K⁺ | 1+ | potassium ion |
| Cs⁺ | 1+ | cesium ion |
| Ag⁺ | 1+ | silver ion |
| Mg²⁺ | 2+ | magnesium ion |
| Ca²⁺ | 2+ | calcium ion |
| Sr²⁺ | 2+ | strontium ion |
| Ba²⁺ | 2+ | barium ion |
| Zn²⁺ | 2+ | zinc ion |
| Cd²⁺ | 2+ | cadmium ion |
| Al³⁺ | 3+ | aluminum ion |

Polyatomic Cations

| | | |
|---|---|---|
| NH₄⁺ | 1+ | ammonium ion |
| H₃O⁺ | 1+ | hydronium ion |

Multivalent Cations

| | | |
|---|---|---|
| Cr²⁺ | 2 | chromium (II) or chromous ion |
| Cr³⁺ | 3 | chromium (III)or chromic ion |
| Mn²⁺ | 2 | manganese (II) or manganous ion |
| Mn⁴⁺ | 4 | manganese (IV) ion |
| Fe²⁺ | 2 | iron (II) or ferrous ion |
| Fe³⁺ | 3 | iron (III) or ferric ion |
| Co²⁺ | 2 | cobalt (II) or cobaltous ion |
| Co³⁺ | 3 | cobalt (II) or cobaltic ion |
| Ni²⁺ | 2 | nickel (II) or nickelous ion |
| Ni³⁺ | 3 | nickel (III) or nickelic ion |
| Cu⁺ | 1 | copper (I) or cuprous ion |
| Cu²⁺ | 2 | copper (II) or cupric ion |
| Sn²⁺ | 2 | tin (II) or atannous ion |
| Sn⁴⁺ | 4 | tin (IV) or atannic ion |
| Pb²⁺ | 2 | lead (II) or plumbous ion |
| Pb⁴⁺ | 4 | lead (IV) or plumbic ion |

Monoatomic Anions

| | | |
|---|---|---|
| H⁻ | 1− | hydride ion |
| F⁻ | 1− | fluoride ion |
| Cl⁻ | 1− | chloride ion |
| Br⁻ | 1− | bromide ion |
| I⁻ | 1− | iodide ion |
| O²⁻ | 2− | oxide ion |
| S²⁻ | 2− | sulfide ion |
| N³⁻ | 3− | nitride ion |

Polyatomic Anions

| | | |
|---|---|---|
| OH⁻ | 1− | hydroxide ion |
| CN⁻ | 1− | cyanide ion |
| SCN⁻ | 1− | thiocyanate ion |
| C₂H₃O₂⁻ | 1− | acetate ion |
| ClO⁻ | 1− | hypochlorite ion |
| ClO₂⁻ | 1− | chlorite ion |
| ClO₃⁻ | 1− | chlorate ion |
| ClO₄⁻ | 1− | perchlorate ion |
| NO₂⁻ | 1− | nitrite ion |
| NO₃⁻ | 1− | nitrate ion |
| MnO₄²⁻ | 2− | permanganate ion |
| CO₃²⁻ | 2− | carbonate ion |
| C₂O₄²⁻ | 2− | oxalate ion |
| CrO₄²⁻ | 2− | chromate ion |
| Cr₂O₇²⁻ | 2− | dichromate ion |
| SO₃²⁻ | 2− | sulfite ion |
| SO₄²⁻ | 2− | sulfate ion |
| PO₃³⁻ | 3− | phosphite ion |
| PO₄³⁻ | 3− | phosphate ion |

The present disclosure sets forth novel gas-enriched fluids, including, but not limited to gas-enriched ionic aqueous solutions, aqueous saline solutions (e.g., standard aqueous saline solutions, and other saline solutions as discussed herein and as would be recognized in the art, including any physiological compatible saline solutions), cell culture media (e.g., minimal medium, and other culture media) useful in the treatment of diabetes or diabetes related disorders. A medium, or media, is termed “minimal” if it only contains the nutrients essential for growth. For prokaryotic host cells, a minimal media typically includes a source of carbon, nitrogen, phosphorus, magnesium, and trace amounts of iron and calcium. (Gunsalus and Stanter, The Bacteria, V. 1, Ch. 1 Acad. Press Inc., N.Y. (1960)). Most minimal media use glucose as a carbon source, ammonia as a nitrogen source, and orthophosphate (e.g., PO₄) as the phosphorus source. The media components can be varied or supplemented according to the specific prokaryotic or eukaryotic organism(s) grown, in order to encourage optimal growth without inhibiting target protein production. (Thompson et al., Biotech. and Bioeng. 27: 818-824 (1985)).

In particular aspects, the electrokinetically altered aqueous fluids are suitable to modulate ¹³C-NMR line-widths of reporter solutes (e.g., Trehelose) dissolved therein. NMR line-width effects are in indirect method of measuring, for example, solute ‘tumbling’ in a test fluid as described herein in particular working Examples.

In particular aspects, the electrokinetically altered aqueous fluids are characterized by at least one of: distinctive square wave voltametry peak differences at any one of −0.14V, −0.47V, −1.02V and −1.36V; polarographic peaks at −0.9 volts; and an absence of polarographic peaks at −0.19 and −0.3 volts, which are unique to the electrokinetically generated fluids as disclosed herein in particular working Examples.

In particular aspects, the electrokinetically altered aqueous fluids are suitable to alter cellular membrane conductivity (e.g., a voltage-dependent contribution of the whole-cell conductance as measure in patch clamp studies disclosed herein).

In particular aspects, the electrokinetically altered aqueous fluids are oxygenated, wherein the oxygen in the fluid is present in an amount of at least 15, ppm, at least 25 ppm, at least 30 ppm, at least 40 ppm, at least 50 ppm, or at least 60 ppm dissolved oxygen at atmospheric pressure. In particular aspects, the electrokinetically altered aqueous fluids have less than 15 ppm, less that 10 ppm of dissolved oxygen at atmospheric pressure, or approximately ambient oxygen levels.

In particular aspects, the electrokinetically altered aqueous fluids are oxygenated, wherein the oxygen in the fluid is present in an amount between approximately 8 ppm and approximately 15 ppm, and in this case is sometimes referred to herein as “Solas.”

In particular aspects, the electrokinetically altered aqueous fluid comprises at least one of solvated electrons (e.g., stabilized by molecular oxygen), and electrokinetically modified and/or charged oxygen species, and wherein in certain embodiments the solvated electrons and/or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm.

In particular aspects, the electrokinetically altered aqueous fluids are characterized by differential (e.g., increased or decreased) permittivity relative to control, non-electrokinetically altered fluids. In preferred aspects, the electrokinetically altered aqueous fluids are characterized by differential, increased permittivity relative to control, non-electrokinetically altered fluids. Permittivity (∈) (farads per meter) is a measure of the ability of a material to be polarized by an electric field and thereby reduce the total electric field inside the material. Thus, permittivity relates to a material's ability to transmit (or “permit”) an electric field. Capacitance (C) (farad; coulomb per volt), a closely related property, is a measure of the ability of a material to hold charge if a voltage is applied across it (e.g., best modeled by a dielectric layer sandwiched between two parallel conductive plates). If a voltage V is applied across a capacitor of capacitance C, then the charge Q that it can hold is directly proportional to the applied voltage V, with the capacitance C as the proportionality constant. Thus, Q=CV, or C=Q/V. The capacitance of a capacitor depends on the permittivity ∈ of the dielectric layer, as well as the area A of the capacitor and the separation distance d between the two conductive plates. Permittivity and capacitance are mathematically related as follows: C=∈(A/d). When the dielectric used is vacuum, then the capacitance Co=∈o (A/d), where ∈o is the permittivity of vacuum (8.85×10⁻¹² F/m). The dielectric constant (k), or relative permittivity of a material is the ratio of its permittivity ∈ to the permittivity of vacuum ∈o, so k=∈/∈o (the dielectric constant of vacuum is 1). A low-k dielectric is a dielectric that has a low permittivity, or low ability to polarize and hold charge. A high-k dielectric, on the other hand, has a high permittivity. Because high-k dielectrics are good at holding charge, they are the preferred dielectric for capacitors. High-k dielectrics are also used in memory cells that store digital data in the form of charge.

In particular aspects, the electrokinetically altered aqueous fluids are suitable to alter cellular membrane structure or function (e.g., altering of a conformation, ligand binding activity, or a catalytic activity of a membrane associated protein) sufficient to provide for modulation of intracellular signal transduction, wherein in particular aspects, the membrane associated protein comprises at least one selected from the group consisting of receptors, transmembrane receptors (e.g., G-Protein Coupled Receptor (GPCR), TSLP receptor, beta 2 adrenergic receptor, bradykinin receptor, etc.), ion channel proteins, intracellular attachment proteins, cellular adhesion proteins, and integrins. In certain aspects, the effected G-Protein Coupled Receptor (GPCR) interacts with a G protein a subunit (e.g., Gαₛ, Gαᵢ, Gα_{q}, and Gα₁₂).

In particular aspects, the electrokinetically altered aqueous fluids are suitable to modulate intracellular signal transduction, comprising modulation of a calcium dependent cellular messaging pathway or system (e.g., modulation of phospholipase C activity, or modulation of adenylate cyclase (AC) activity).

In particular aspects, the electrokinetically altered aqueous fluids are characterized by various biological activities (e.g., regulation of cytokines, receptors, enzymes and other proteins and intracellular signaling pathways) described in the working Examples and elsewhere herein.

In particular aspects, the electrokinetically altered aqueous fluids display synergy with glatiramer acetate interferon-β, mitoxantrone, and/or natalizumab. In particular aspects, the electrokinetically altered aqueous fluids reduce DEP-induced TSLP receptor expression in bronchial epithelial cells (BEC).

In particular aspects, the electrokinetically altered aqueous fluids inhibit the DEP-induced cell surface-bound MMP9 levels in bronchial epithelial cells (BEC).

In particular aspects, the biological effects of the electrokinetically altered aqueous fluids are inhibited by diphtheria toxin, indicating that beta blockade, GPCR blockade and Ca channel blockade affects the activity of the electrokinetically altered aqueous fluids (e.g., on regulatory T cell function).

In particular aspects, the physical and biological effects (e.g., the ability to alter cellular membrane structure or function sufficient to provide for modulation of intracellular signal transduction) of the electrokinetically altered aqueous fluids persists for at least two, at least three, at least four, at least five, at least 6 months, or longer periods, in a closed container (e.g., closed gas-tight container at atmospheric pressure; and preferable at 4 degrees C.).

According to particular aspects, the charge-stabilized oxygen containing nanostructures (nanobubbles) having an average diameter of less than 100 nm of the electrokinetically altered aqueous fluids persist for at least two, at least three, at least four, at least five, at least 6 months, or longer periods, in a closed container (e.g., closed gas-tight container at atmospheric pressure; and preferable at 4 degrees C.), which accounts for, and correlates with the stability of the biological activity of the fluid.

Therefore, further aspects provide said electrokinetically-generated solutions and methods of producing an electrokinetically altered oxygenated aqueous fluid or solution, comprising: providing a flow of a fluid material between two spaced surfaces in relative motion and defining a mixing volume therebetween, wherein the dwell time of a single pass of the flowing fluid material within and through the mixing volume is greater than 0.06 seconds or greater than 0.1 seconds; and introducing oxygen (O₂) into the flowing fluid material within the mixing volume under conditions suitable to dissolve at least 20 ppm, at least 25 ppm, at least 30, at least 40, at least 50, or at least 60 ppm oxygen into the material, and electrokinetically alter the fluid or solution. In certain aspects, the oxygen is infused into the material in less than 100 milliseconds, less than 200 milliseconds, less than 300 milliseconds, or less than 400 milliseconds. In particular embodiments, the ratio of surface area to the volume is at least 12, at least 20, at least 30, at least 40, or at least 50.

Yet further aspects, provide a method of producing an electrokinetically altered oxygenated aqueous fluid or solution, comprising: providing a flow of a fluid material between two spaced surfaces defining a mixing volume therebetween; and introducing oxygen into the flowing material within the mixing volume under conditions suitable to infuse at least 20 ppm, at least 25 ppm, at least 30, at least 40, at least 50, or at least 60 ppm oxygen into the material in less than 100 milliseconds, less than 200 milliseconds, less than 300 milliseconds, or less than 400 milliseconds. In certain aspects, the dwell time of the flowing material within the mixing volume is greater than 0.06 seconds or greater than 0.1 seconds. In particular embodiments, the ratio of surface area to the volume is at least 12, at least 20, at least 30, at least 40, or at least 50.

Additional embodiments provide a method of producing an electrokinetically altered oxygenated aqueous fluid or solution, comprising use of a mixing device for creating an output mixture by mixing a first material and a second material, the device comprising: a first chamber configured to receive the first material from a source of the first material; a stator; a rotor having an axis of rotation, the rotor being disposed inside the stator and configured to rotate about the axis of rotation therein, at least one of the rotor and stator having a plurality of through-holes; a mixing chamber defined between the rotor and the stator, the mixing chamber being in fluid communication with the first chamber and configured to receive the first material therefrom, and the second material being provided to the mixing chamber via the plurality of through-holes formed in the one of the rotor and stator; a second chamber in fluid communication with the mixing chamber and configured to receive the output material therefrom; and a first internal pump housed inside the first chamber, the first internal pump being configured to pump the first material from the first chamber into the mixing chamber. In certain aspects, the first internal pump is configured to impart a circumferential velocity into the first material before it enters the mixing chamber.

Further embodiments provide a method of producing an electrokinetically altered oxygenated aqueous fluid or solution, comprising use of a mixing device for creating an output mixture by mixing a first material and a second material, the device comprising: a stator; a rotor having an axis of rotation, the rotor being disposed inside the stator and configured to rotate about the axis of rotation therein; a mixing chamber defined between the rotor and the stator, the mixing chamber having an open first end through which the first material enters the mixing chamber and an open second end through which the output material exits the mixing chamber, the second material entering the mixing chamber through at least one of the rotor and the stator; a first chamber in communication with at least a majority portion of the open first end of the mixing chamber; and a second chamber in communication with the open second end of the mixing chamber.

Additional aspects provide an electrokinetically altered oxygenated aqueous fluid or solution made according to any of the above methods. In particular aspects the administered inventive electrokinetically-altered fluids comprise charge-stabilized oxygen-containing nanostructures in an amount sufficient to provide modulation of at least one of cellular membrane potential and cellular membrane conductivity. In certain embodiments, the electrokinetically-altered fluids are superoxygenated (e.g., RNS-20, RNS-40 and RNS-60, comprising 20 ppm, 40 ppm and 60 ppm dissolved oxygen, respectively, in standard saline). In particular embodiments, the electrokinetically-altered fluids are not-superoxygenated (e.g., RNS-10 or Solas, comprising 10 ppm (e.g., approx. ambient levels of dissolved oxygen in standard saline). In certain aspects, the salinity, sterility, pH, etc., of the inventive electrokinetically-altered fluids is established at the time of electrokinetic production of the fluid, and the sterile fluids are administered by an appropriate route. Alternatively, at least one of the salinity, sterility, pH, etc., of the fluids is appropriately adjusted (e.g., using sterile saline or appropriate diluents) to be physiologically compatible with the route of administration prior to administration of the fluid. Preferably, and diluents and/or saline solutions and/or buffer compositions used to adjust at least one of the salinity, sterility, pH, etc., of the fluids are also electrokinetic fluids, or are otherwise compatible therewith.

The present disclosure sets forth novel gas-enriched fluids, including, but not limited to gas-enriched ionic aqueous solutions, aqueous saline solutions (e.g., standard aqueous saline solutions, and other saline solutions as discussed herein and as would be recognized in the art, including any physiological compatible saline solutions), cell culture media (e.g., minimal medium, and other culture media).

According to particular aspects of the methods and fluids above, the charge-stabilized oxygen-containing nanostructures comprise charge-stabilized oxygen-containing nanobubbles predominantly having an average diameter less than 100 nm. According to particular aspects, the charge-stabilized oxygen-containing nanobubbles are stable to persist in solution for at least months in a closed container at atmospheric pressure.

### Methods of Treatment

The term “treating” or “administering” refers to, and includes, reversing, alleviating, inhibiting the progress of, or preventing a disease, disorder or condition, or one or more symptoms thereof; and “treatment” and “therapeutically” refer to the act of treating, as defined herein.

A “therapeutically effective amount” is any amount of any of the compounds utilized in the course of practicing the invention provided herein that is sufficient to reverse, alleviate, inhibit the progress of, or prevent a disease, disorder or condition, or one or more symptoms thereof.

Certain embodiments herein relate to therapeutic compositions and methods of treatment for a subject by enhancing hippocampal plasticity and hippocampal-mediated learning and memory, as disclosed herein.

### Combination Therapy:

Additional aspects provide the herein disclosed inventive methods, further comprising combination therapy, wherein at least one additional therapeutic agent is administered to the patient. In certain aspects, the at least one additional therapeutic agent is and anti-inflammatory agent, as disclosed herein.

### Exemplary Relevant Molecular Interactions:

Conventionally, quantum properties are thought to belong to elementary particles of less than 10⁻¹⁰ meters, while the macroscopic world of our everyday life is referred to as classical, in that it behaves according to Newton's laws of motion.

Recently, molecules have been described as forming clusters that increase in size with dilution. These clusters measure several micrometers in diameter, and have been reported to increase in size non-linearly with dilution. Quantum coherent domains measuring 100 nanometers in diameter have been postulated to arise in pure water, and collective vibrations of water molecules in the coherent domain may eventually become phase locked to electromagnetic field fluctuations, providing for stable oscillations in water, providing a form of ‘memory’ in the form of excitation of long lasting coherent oscillations specific to dissolved substances in the water that change the collective structure of the water, which may in turn determine the specific coherent oscillations that develop. Where these oscillations become stabilized by magnetic field phase coupling, the water, upon dilution may still carry ‘seed’ coherent oscillations. As a cluster of molecules increases in size, its electromagnetic signature is correspondingly amplified, reinforcing the coherent oscillations carried by the water.

Despite variations in the cluster size of dissolved molecules and detailed microscopic structure of the water, a specificity of coherent oscillations may nonetheless exist. One model for considering changes in properties of water is based on considerations involved in crystallization.

A protonated water cluster typically takes the form of H⁺(H₂0)ₙ. Some protonated water clusters occur naturally, such as in the ionosphere. Without being bound by any particular theory, and according to particular aspects, other types of water clusters or structures (nanoclusters, nanocages, nanobubbles) are possible, including nanostructures comprising oxygen (and possibly stabilized electrons imparted to the inventive output materials). Oxygen atoms may be caught in the resulting structures. The chemistry of the semi-bound nanocage or nanobubble allows the oxygen and/or stabilized electrons to remain dissolved for extended periods of time. Other atoms or molecules, such as medicinal compounds, can be combined for sustained delivery purposes. The specific chemistry of the solution material and dissolved compounds depend on the interactions of those materials.

As described previously in Applicants' WO 2009/055729, “Double Layer Effect,” “Dwell Time,” “Rate of Infusion,” and “Bubble size Measurements,” the electrokinetic mixing device creates, in a matter of milliseconds, a unique non-linear fluid dynamic interaction of the first material and the second material with complex, dynamic turbulence providing complex mixing in contact with an effectively enormous surface area (including those of the device and of the exceptionally small gas bubbles; nanobubbles of less than 100 nm) that provides for the novel therapeutic effects described herein. Additionally, feature-localized electrokinetic effects (voltage/current) were demonstrated using a specially designed mixing device comprising insulated rotor and stator features (also see, e.g., Applicants' issued U.S. Pat. Nos. 7,832,920, 7,919,534, 8,410,182, 8,445,546, 8,449,172, and 8,470,893, all incorporated herein by reference in their respective entireties).

As well-recognized in the art, charge redistributions and/or solvated electrons are known to be highly unstable in aqueous solution. According to particular aspects, Applicants' electrokinetic effects (e.g., charge redistributions, including, in particular aspects, solvated electrons) are surprisingly stabilized within the output material (e.g., saline solutions, ionic solutions). In fact, as described herein, the stability of the properties and biological activity of the inventive electrokinetic fluids (e.g., RNS-60 or Solas (processed through device but with no added Oxygen) can be maintained for months in a gas-tight container, indicating involvement of dissolved gas (e.g., oxygen) in helping to generate and/or maintain, and/or mediate the properties and activities of the inventive solutions. Significantly, the charge redistributions and/or solvated electrons are stably configured in the inventive electrokinetic ionic aqueous fluids in an amount sufficient to provide, upon contact with a living cell (e.g., mammalian cell) by the fluid, modulation of at least one of cellular membrane potential and cellular membrane conductivity (see, e.g., cellular patch clamp working Example 23 from WO 2009/055729 and as disclosed herein).

As described herein under “Molecular Interactions,” to account for the stability and biological compatibility of the inventive electrokinetic fluids (e.g., electrokinetic saline solutions), Applicants have proposed that interactions between the water molecules and the molecules of the substances (e.g., oxygen) dissolved in the water change the collective structure of the water and provide for nanoscale structures (e.g., nanobubbles), including nanostructure (e.g., nanobubbles) comprising oxygen and/or stabilized electrons imparted to the inventive output materials. Without being bound by mechanism, the configuration of the nanostructures (e.g., nanobubbles) in particular aspects is such that they: comprise (at least for formation and/or stability and/or biological activity) dissolved gas (e.g., oxygen); enable the electrokinetic fluids (e.g., RNS-60 or Solas saline fluids) to modulate (e.g., impart or receive) charges and/or charge effects upon contact with a cell membrane or related constituent thereof; and in particular aspects provide for stabilization (e.g., carrying, harboring, trapping) solvated electrons in a biologically-relevant form.

According to particular aspects, and as supported by the present disclosure, in ionic or saline (e.g., standard saline, NaCl) solutions, the inventive nanostructures comprise charge stabilized nanostructures (e.g., nanobubbles) (e.g., average diameter less that 100 nm) that may comprise at least one dissolved gas molecule (e.g., oxygen) within a charge-stabilized hydration shell. According to additional aspects, the charge-stabilized hydration shell may comprise a cage or void harboring the at least one dissolved gas molecule (e.g., oxygen). According to further aspects, by virtue of the provision of suitable charge-stabilized hydration shells, the charge-stabilized nanostructure and/or charge-stabilized oxygen-containing nanostructures may additionally comprise a solvated electron (e.g., stabilized solvated electron).

According to particular aspects of the present invention, Applicants' novel electrokinetic fluids comprise a novel, biologically active form of charge-stabilized oxygen-containing nanostructures (e.g., nanobubbles), and may further comprise novel arrays, clusters or associations of such structures (e.g., of such nanobubbles).

According to a charge-stabilized microbubble model, the short-range molecular order of the water structure is destroyed by the presence of a gas molecule (e.g., a dissolved gas molecule initially complexed with a nonadsorptive ion provides a short-range order defect), providing for condensation of ionic droplets, wherein the defect is surrounded by first and second coordination spheres of water molecules, which are alternately filled by adsorptive ions (e.g., acquisition of a ‘screening shell of Na⁺ ions to form an electrical double layer) and nonadsorptive ions (e.g., Cl⁻ ions occupying the second coordination sphere) occupying six and 12 vacancies, respectively, in the coordination spheres. In under-saturated ionic solutions (e.g., undersaturated saline solutions), this hydrated ‘nucleus’ remains stable until the first and second spheres are filled by six adsorptive and five nonadsorptive ions, respectively, and then undergoes Coulomb explosion creating an internal void containing the gas molecule, wherein the adsorptive ions (e.g., Na⁺ ions) are adsorbed to the surface of the resulting void, while the nonadsorptive ions (or some portion thereof) diffuse into the solution (Bunkin et al., supra). In this model, the void in the nanostructure is prevented from collapsing by Coulombic repulsion between the ions (e.g., Na⁺ ions) adsorbed to its surface. The stability of the void-containing nanostructures is postulated to be due to the selective adsorption of dissolved ions with like charges onto the void/bubble surface and diffusive equilibrium between the dissolved gas and the gas inside the bubble, where the negative (outward electrostatic pressure exerted by the resulting electrical double layer provides stable compensation for surface tension, and the gas pressure inside the bubble is balanced by the ambient pressure. According to the model, formation of such microbubbles requires an ionic component, and in certain aspects collision-mediated associations between particles may provide for formation of larger order clusters (arrays) (Id).

The charge-stabilized microbubble model suggests that the particles can be gas microbubbles, but contemplates only spontaneous formation of such structures in ionic solution in equilibrium with ambient air, is uncharacterized and silent as to whether oxygen is capable of forming such structures, and is likewise silent as to whether solvated electrons might be associated and/or stabilized by such structures.

According to particular aspects, the inventive electrokinetic fluids comprising charge-stabilized nanostructures and/or charge-stabilized oxygen-containing nanostructures are novel and fundamentally distinct from the postulated non-electrokinetic, atmospheric charge-stabilized microbubble structures according to the microbubble model. Significantly, this conclusion is unavoidable, deriving, at least in part, from the fact that control saline solutions do not have the biological properties disclosed herein, whereas Applicants' charge-stabilized nanostructures provide a novel, biologically active form of charge-stabilized oxygen-containing nanostructures.

According to particular aspects of the present invention, Applicants' novel electrokinetic device and methods provide for novel electrokinetically-altered fluids comprising significant quantities of charge-stabilized nanostructures in excess of any amount that may or may not spontaneously occur in ionic fluids in equilibrium with air, or in any non-electrokinetically generated fluids. In particular aspects, the charge-stabilized nanostructures comprise charge-stabilized oxygen-containing nanostructures. In additional aspects, the charge-stabilized nanostructures are all, or substantially all charge-stabilized oxygen-containing nanostructures, or the charge-stabilized oxygen-containing nanostructures the major charge-stabilized gas-containing nanostructure species in the electrokinetic fluid.

According to yet further aspects, the charge-stabilized nanostructures and/or the charge-stabilized oxygen-containing nanostructures may comprise or harbor a solvated electron, and thereby provide a novel stabilized solvated electron carrier. In particular aspects, the charge-stabilized nanostructures and/or the charge-stabilized oxygen-containing nanostructures provide a novel type of electride (or inverted electride), which in contrast to conventional solute electrides having a single organically coordinated cation, rather have a plurality of cations stably arrayed about a void or a void containing an oxygen atom, wherein the arrayed sodium ions are coordinated by water hydration shells, rather than by organic molecules. According to particular aspects, a solvated electron may be accommodated by the hydration shell of water molecules, or preferably accommodated within the nanostructure void distributed over all the cations. In certain aspects, the inventive nanostructures provide a novel ‘super electride’ structure in solution by not only providing for distribution/stabilization of the solvated electron over multiple arrayed sodium cations, but also providing for association or partial association of the solvated electron with the caged oxygen molecule(s) in the void—the solvated electron distributing over an array of sodium atoms and at least one oxygen atom. According to particular aspects, therefore, ‘solvated electrons’ as presently disclosed in association with the inventive electrokinetic fluids, may not be solvated in the traditional model comprising direct hydration by water molecules. Alternatively, in limited analogy with dried electride salts, solvated electrons in the inventive electrokinetic fluids may be distributed over multiple charge-stabilized nanostructures to provide a ‘lattice glue’ to stabilize higher order arrays in aqueous solution.

In particular aspects, the inventive charge-stabilized nanostructures and/or the charge-stabilized oxygen-containing nanostructures are capable of interacting with cellular membranes or constituents thereof, or proteins, etc., to mediate biological activities. In particular aspects, the inventive charge-stabilized nanostructures and/or the charge-stabilized oxygen-containing nanostructures harboring a solvated electron are capable of interacting with cellular membranes or constituents thereof, or proteins, etc., to mediate biological activities.

In particular aspects, the inventive charge-stabilized nanostructures and/or the charge-stabilized oxygen-containing nanostructures interact with cellular membranes or constituents thereof, or proteins, etc., as a charge and/or charge effect donor (delivery) and/or as a charge and/or charge effect recipient to mediate biological activities. In particular aspects, the inventive charge-stabilized nanostructures and/or the charge-stabilized oxygen-containing nanostructures harboring a solvated electron interact with cellular membranes as a charge and/or charge effect donor and/or as a charge and/or charge effect recipient to mediate biological activities.

In particular aspects, the inventive charge-stabilized nanostructures and/or the charge-stabilized oxygen-containing nanostructures are consistent with, and account for the observed stability and biological properties of the inventive electrokinetic fluids.

In particular aspects, the charge-stabilized oxygen-containing nanostructures substantially comprise, take the form of, or can give rise to, charge-stabilized oxygen-containing nanobubbles. In particular aspects, charge-stabilized oxygen-containing clusters provide for formation of relatively larger arrays of charge-stabilized oxygen-containing nanostructures, and/or charge-stabilized oxygen-containing nanobubbles or arrays thereof. In particular aspects, the charge-stabilized oxygen-containing nanostructures can provide for formation of hydrophobic nanobubbles upon contact with a hydrophobic surface.

In particular aspects, the charge-stabilized oxygen-containing nanostructures substantially comprise at least one oxygen molecule. In certain aspects, the charge-stabilized oxygen-containing nanostructures substantially comprise at least 1, at least 2, at least 3, at least 4, at least 5, at least 10 at least 15, at least 20, at least 50, at least 100, or greater oxygen molecules. In particular aspects, charge-stabilized oxygen-containing nanostructures comprise or give rise to nanobubbles (e.g., hydrophobid nanobubbles) of about 20 nm×1.5 nm, comprise about 12 oxygen molecules (e.g., based on the size of an oxygen molecule (approx 0.3 nm by 0.4 nm), assumption of an ideal gas and application of n=PV/RT, where P=1 atm, R=0.082 057 l·atm/mol·K; T=295K; V=pr²h=4.7×10⁻²² L, where r=10×10⁻⁹ m, h=1.5×10⁻⁹ m, and n=1.95×10⁻²² moles).

In certain aspects, the percentage of oxygen molecules present in the fluid that are in such nanostructures, or arrays thereof, having a charge-stabilized configuration in the ionic aqueous fluid is a percentage amount selected from the group consisting of greater than: 0.1%, 1%; 2%; 5%; 10%; 15%; 20%; 25%; 30%; 35%; 40%; 45%; 50%; 55%; 60%; 65%; 70%; 75%; 80%; 85%; 90%; and greater than 95%. Preferably, this percentage is greater than about 5%, greater than about 10%, greater than about 15% f, or greater than about 20%. In additional aspects, the substantial size of the charge-stabilized oxygen-containing nanostructures, or arrays thereof, having a charge-stabilized configuration in the ionic aqueous fluid is a size selected from the group consisting of less than: 100 nm; 90 nm; 80 nm; 70 nm; 60 nm; 50 nm; 40 nm; 30 nm; 20 nm; 10 nm; 5 nm; 4 nm; 3 nm; 2 nm; and 1 nm. Preferably, this size is less than about 50 nm, less than about 40 nm, less than about 30 nm, less than about 20 nm, or less than about 10 nm.

In certain aspects, the inventive electrokinetic fluids comprise solvated electrons. In further aspects, the inventive electrokinetic fluids comprises charge-stabilized nanostructures and/or charge-stabilized oxygen-containing nanostructures, and/or arrays thereof, which comprise at least one of: solvated electron(s); and unique charge distributions (polar, symmetric, asymmetric charge distribution). In certain aspects, the charge-stabilized nanostructures and/or charge-stabilized oxygen-containing nanostructures, and/or arrays thereof, have paramagnetic properties.

By contrast, relative to the inventive electrokinetic fluids, control pressure pot oxygenated fluids (non-electrokinetic fluids) and the like do not comprise such electrokinetically generated charge-stabilized biologically-active nanostructures and/or biologically-active charge-stabilized oxygen-containing nanostructures and/or arrays thereof, capable of modulation of at least one of cellular membrane potential and cellular membrane conductivity.

### Routes and Forms of Administration

In particular exemplary embodiments, the gas-enriched fluid of the present invention may function as a therapeutic composition alone or in combination with another therapeutic agent such that the therapeutic composition enhances hippocampal plasticity and hippocampal-mediated learning and memory. The therapeutic compositions of the present invention include compositions that are able to be administered to a subject in need thereof. In certain embodiments, the therapeutic composition formulation may also comprise at least one additional agent selected from the group consisting of: carriers, adjuvants, emulsifying agents, suspending agents, sweeteners, flavorings, perfumes, and binding agents.

As used herein, “pharmaceutically acceptable carrier” and “carrier” generally refer to a non-toxic, inert solid, semi-solid or liquid filler, diluent, encapsulating material or formulation auxiliary of any type. Some non-limiting examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. In particular aspects, such carriers and excipients may be gas-enriched fluids or solutions of the present invention.

The pharmaceutically acceptable carriers described herein, for example, vehicles, adjuvants, excipients, or diluents, are well known to those who are skilled in the art. Typically, the pharmaceutically acceptable carrier is chemically inert to the therapeutic agents and has no detrimental side effects or toxicity under the conditions of use. The pharmaceutically acceptable carriers can include polymers and polymer matrices, nanoparticles, microbubbles, and the like.

In addition to the therapeutic gas-enriched fluid of the present invention, the therapeutic composition may further comprise inert diluents such as additional non-gas-enriched water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. As is appreciated by those of ordinary skill, a novel and improved formulation of a particular therapeutic composition, a novel gas-enriched therapeutic fluid, and a novel method of delivering the novel gas-enriched therapeutic fluid may be obtained by replacing one or more inert diluents with a gas-enriched fluid of identical, similar, or different composition. For example, conventional water may be replaced or supplemented by a gas-enriched fluid produced by mixing oxygen into water or deionized water to provide gas-enriched fluid.

In certain embodiments, the inventive gas-enriched fluid may be combined with one or more therapeutic agents and/or used alone. In particular embodiments, incorporating the gas-enriched fluid may include replacing one or more solutions known in the art, such as deionized water, saline solution, and the like with one or more gas-enriched fluid, thereby providing an improved therapeutic composition for delivery to the subject.

Certain embodiments provide for therapeutic compositions comprising a gas-enriched fluid of the present invention, a pharmaceutical composition or other therapeutic agent or a pharmaceutically acceptable salt or solvate thereof, and at least one pharmaceutical carrier or diluent. These pharmaceutical compositions may be used in the prophylaxis and treatment of the foregoing diseases or conditions and in therapies as mentioned above. Preferably, the carrier must be pharmaceutically acceptable and must be compatible with, i.e. not have a deleterious effect upon, the other ingredients in the composition. The carrier may be a solid or liquid and is preferably formulated as a unit dose formulation, for example, a tablet that may contain from 0.05 to 95% by weight of the active ingredient.

Possible administration routes include oral, sublingual, buccal, parenteral (for example subcutaneous, intramuscular, intra-arterial, intraperitoneally, intracisternally, intravesically, intrathecally, or intravenous), rectal, topical including transdermal, intravaginal, intraoccular, intraotical, intranasal, inhalation, and injection or insertion of implantable devices or materials.

### Administration Routes

Most suitable means of administration for a particular subject will depend on the nature and severity of the disease or condition being treated or the nature of the therapy being used, as well as the nature of the therapeutic composition or additional therapeutic agent. In certain embodiments, oral or topical administration is preferred.

Formulations suitable for oral administration may be provided as discrete units, such as tablets, capsules, cachets, syrups, elixirs, chewing gum, “lollipop” formulations, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Additional formulations suitable for oral administration may be provided to include fine particle dusts or mists which may be generated by means of various types of metered dose pressurized aerosols, atomizers, nebulisers, or insufflators. In particular, powders or other compounds of therapeutic agents may be dissolved or suspended in a gas-enriched fluid of the present invention.

Formulations suitable for transmucosal methods, such as by sublingual or buccal administration include lozenges patches, tablets, and the like comprising the active compound and, typically a flavored base, such as sugar and acacia or tragacanth and pastilles comprising the active compound in an inert base, such as gelatin and glycerine or sucrose acacia.

Formulations suitable for parenteral administration typically comprise sterile aqueous solutions containing a predetermined concentration of the active gas-enriched fluid and possibly another therapeutic agent; the solution is preferably isotonic with the blood of the intended recipient. Additional formulations suitable for parenteral administration include formulations containing physiologically suitable co-solvents and/or complexing agents such as surfactants and cyclodextrins. Oil-in-water emulsions may also be suitable for formulations for parenteral administration of the gas-enriched fluid. Although such solutions are preferably administered intravenously, they may also be administered by subcutaneous or intramuscular injection.

Formulations suitable for urethral, rectal or vaginal administration include gels, creams, lotions, aqueous or oily suspensions, dispersible powders or granules, emulsions, dissolvable solid materials, douches, and the like. The formulations are preferably provided as unit-dose suppositories comprising the active ingredient in one or more solid carriers forming the suppository base, for example, cocoa butter. Alternatively, colonic washes with the gas-enriched fluids of the present invention may be formulated for colonic or rectal administration.

Formulations suitable for topical, intraoccular, intraotic, or intranasal application include ointments, creams, pastes, lotions, pastes, gels (such as hydrogels), sprays, dispersible powders and granules, emulsions, sprays or aerosols using flowing propellants (such as liposomal sprays, nasal drops, nasal sprays, and the like) and oils. Suitable carriers for such formulations include petroleum jelly, lanolin, polyethyleneglycols, alcohols, and combinations thereof. Nasal or intranasal delivery may include metered doses of any of these formulations or others. Likewise, intraotic or intraocular may include drops, ointments, irritation fluids and the like.

Formulations of the invention may be prepared by any suitable method, typically by uniformly and intimately admixing the gas-enriched fluid optionally with an active compound with liquids or finely divided solid carriers or both, in the required proportions and then, if necessary, shaping the resulting mixture into the desired shape.

For example a tablet may be prepared by compressing an intimate mixture comprising a powder or granules of the active ingredient and one or more optional ingredients, such as a binder, lubricant, inert diluent, or surface active dispersing agent, or by molding an intimate mixture of powdered active ingredient and a gas-enriched fluid of the present invention.

Suitable formulations for administration by inhalation include fine particle dusts or mists which may be generated by means of various types of metered dose pressurized aerosols, atomizers, nebulisers, or insufflators. In particular, powders or other compounds of therapeutic agents may be dissolved or suspended in a gas-enriched fluid of the present invention.

For pulmonary administration via the mouth, the particle size of the powder or droplets is typically in the range 0.5-10 μM, preferably 1-5 μM, to ensure delivery into the bronchial tree. For nasal administration, a particle size in the range 10-500 μM is preferred to ensure retention in the nasal cavity.

Metered dose inhalers are pressurized aerosol dispensers, typically containing a suspension or solution formulation of a therapeutic agent in a liquefied propellant. In certain embodiments, as disclosed herein, the gas-enriched fluids of the present invention may be used in addition to or instead of the standard liquefied propellant. During use, these devices discharge the formulation through a valve adapted to deliver a metered volume, typically from 10 to 150 μL, to produce a fine particle spray containing the therapeutic agent and the gas-enriched fluid. Suitable propellants include certain chlorofluorocarbon compounds, for example, dichlorodifluoromethane, trichlorofluoromethane, dichlorotetrafluoroethane and mixtures thereof.

The formulation may additionally contain one or more co-solvents, for example, ethanol surfactants, such as oleic acid or sorbitan trioleate, anti-oxidants and suitable flavoring agents. Nebulisers are commercially available devices that transform solutions or suspensions of the active ingredient into a therapeutic aerosol mist either by means of acceleration of a compressed gas (typically air or oxygen) through a narrow venturi orifice, or by means of ultrasonic agitation. Suitable formulations for use in nebulisers consist of another therapeutic agent in a gas-enriched fluid and comprising up to 40% w/w of the formulation, preferably less than 20% w/w. In addition, other carriers may be utilized, such as distilled water, sterile water, or a dilute aqueous alcohol solution, preferably made isotonic with body fluids by the addition of salts, such as sodium chloride. Optional additives include preservatives, especially if the formulation is not prepared sterile, and may include methyl hydroxy-benzoate, anti-oxidants, flavoring agents, volatile oils, buffering agents and surfactants.

Suitable formulations for administration by insufflation include finely comminuted powders that may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

In addition to the ingredients specifically mentioned above, the formulations of the present invention may include other agents known to those skilled in the art, having regard for the type of formulation in issue. For example, formulations suitable for oral administration may include flavoring agents and formulations suitable for intranasal administration may include perfumes.

The therapeutic compositions of the invention can be administered by any conventional method available for use in conjunction with pharmaceutical drugs, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg. According to certain aspects daily dosage of active ingredient may be 0.001 liters to 10 liters, with the preferred dose being from about 0.01 liters to 1 liter.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

Ointments, pastes, foams, occlusions, creams and gels also can contain excipients, such as starch, tragacanth, cellulose derivatives, silicones, bentonites, silica acid, and talc, or mixtures thereof. Powders and sprays also can contain excipients such as lactose, talc, silica acid, aluminum hydroxide, and calcium silicates, or mixtures of these substances. Solutions of nanocrystalline antimicrobial metals can be converted into aerosols or sprays by any of the known means routinely used for making aerosol pharmaceuticals. In general, such methods comprise pressurizing or providing a means for pressurizing a container of the solution, usually with an inert carrier gas, and passing the pressurized gas through a small orifice. Sprays can additionally contain customary propellants, such as nitrogen, carbon dioxide, and other inert gases. In addition, microspheres or nanoparticles may be employed with the gas-enriched therapeutic compositions or fluids of the present invention in any of the routes required to administer the therapeutic compounds to a subject.

The injection-use formulations can be presented in unit-dose or multi-dose sealed containers, such as ampules and vials, and can be stored in a freeze-dried (lyophilized) condition requiring only the addition of the sterile liquid excipient, or gas-enriched fluid, immediately prior to use. Extemporaneous injection solutions and suspensions can be prepared from sterile powders, granules, and tablets. The requirements for effective pharmaceutical carriers for injectable compositions are well known to those of ordinary skill in the art. See, for example, Pharmaceutics and Pharmacy Practice, J. B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising a gas-enriched fluid of the invention and optionally, an additional therapeutic and a flavor, usually sucrose and acacia or tragacanth; pastilles comprising a gas-enriched fluid and optional additional therapeutic agent in an inert base, such as gelatin and glycerin, or sucrose and acacia; and mouth washes or oral rinses comprising a gas-enriched fluid and optional additional therapeutic agent in a suitable liquid carrier; as well as creams, emulsions, gels and the like.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to a subject, especially an animal, particularly a human, in the context of the present invention should be sufficient to effect a therapeutic response in the animal over a reasonable time frame. One skilled in the art will recognize that dosage will depend upon a variety of factors including the condition of the animal, the body weight of the animal, as well as the condition being treated. A suitable dose is that which will result in a concentration of the therapeutic composition in a subject that is known to affect the desired response.

The size of the dose also will be determined by the route, timing and frequency of administration as well as the existence, nature, and extent of any adverse side effects that might accompany the administration of the therapeutic composition and the desired physiological effect.

It will be appreciated that the compounds of the combination may be administered: (1) simultaneously by combination of the compounds in a co-formulation or (2) by alternation, i.e., delivering the compounds serially, sequentially, in parallel or simultaneously in separate pharmaceutical formulations. In alternation therapy, the delay in administering the second, and optionally a third active ingredient, should not be such as to lose the benefit of a synergistic therapeutic effect of the combination of the active ingredients. According to certain embodiments by either method of administration (1) or (2), ideally the combination should be administered to achieve the most efficacious results. In certain embodiments by either method of administration (1) or (2), ideally the combination should be administered to achieve peak plasma concentrations of each of the active ingredients. A one pill once-per-day regimen by administration of a combination co-formulation may be feasible for some patients suffering from inflammatory neurodegenerative diseases. According to certain embodiments effective peak plasma concentrations of the active ingredients of the combination will be in the range of approximately 0.001 to 100 μM. Optimal peak plasma concentrations may be achieved by a formulation and dosing regimen prescribed for a particular patient. It will also be understood that the inventive fluids and glatiramer acetate, interferon-beta, mitoxantrone, and/or natalizumab or the physiologically functional derivatives of any thereof, whether presented simultaneously or sequentially, may be administered individually, in multiples, or in any combination thereof. In general, during alternation therapy (2), an effective dosage of each compound is administered serially, where in co-formulation therapy (1), effective dosages of two or more compounds are administered together.

The combinations of the invention may conveniently be presented as a pharmaceutical formulation in a unitary dosage form. A convenient unitary dosage formulation contains the active ingredients in any amount from 1 mg to 1 g each, for example but not limited to, 10 mg to 300 mg. The synergistic effects of the inventive fluid in combination with glatiramer acetate, interferon-beta, mitoxantrone, and/or natalizumab may be realized over a wide ratio, for example 1:50 to 50:1 (inventive fluid: glatiramer acetate, interferon-beta, mitoxantrone, and/or natalizumab). In one embodiment the ratio may range from about 1:10 to 10:1. In another embodiment, the weight/weight ratio of inventive fluid to glatiramer acetate, interferon-beta, mitoxantrone, and/or natalizumab in a co-formulated combination dosage form, such as a pill, tablet, caplet or capsule will be about 1, i.e., an approximately equal amount of inventive fluid and glatiramer acetate, interferon-beta, mitoxantrone, and/or natalizumab. In other exemplary co-formulations, there may be more or less inventive fluid and glatiramer acetate, interferon-beta, mitoxantrone, and/or natalizumab. In one embodiment, each compound will be employed in the combination in an amount at which it exhibits anti-inflammatory activity when used alone. Other ratios and amounts of the compounds of said combinations are contemplated within the scope of the invention.

A unitary dosage form may further comprise inventive fluid and glatiramer acetate, interferon-beta, mitoxantrone, and/or natalizumab, or physiologically functional derivatives of either thereof, and a pharmaceutically acceptable carrier.

It will be appreciated by those skilled in the art that the amount of active ingredients in the combinations of the invention required for use in treatment will vary according to a variety of factors, including the nature of the condition being treated and the age and condition of the patient, and will ultimately be at the discretion of the attending physician or health care practitioner. The factors to be considered include the route of administration and nature of the formulation, the animal's body weight, age and general condition and the nature and severity of the disease to be treated.

It is also possible to combine any two of the active ingredients in a unitary dosage form for simultaneous or sequential administration with a third active ingredient. The three-part combination may be administered simultaneously or sequentially. When administered sequentially, the combination may be administered in two or three administrations. According to certain embodiments the three-part combination of inventive fluid and glatiramer acetate, interferon-beta, mitoxantrone, and/or natalizumab may be administered in any order.

The following examples are meant to be illustrative only and not limiting in any way.

### EXAMPLES

### Example 1

### The Electrokinetically-Altered Fluid Solutions were Determined to Comprise Nanobubbles Having an Average Diameter Less than 100 Nanometers

Experiments were performed with a gas-enriched fluid by using the diffuser of the present invention in order to determine a gas microbubble size limit. The microbubble size limit was established by passing the gas enriched fluid through 0.22 and 0.1 micron filters. In performing these tests, a volume of fluid passed through the diffuser of the present invention and generated a gas-enriched fluid. Sixty milliliters of this fluid was drained into a 60 ml syringe. The dissolved oxygen level of the fluid within the syringe was then measured by Winkler titration. The fluid within the syringe was injected through a 0.22 micron Millipore Millex GP50 filter and into a 50 ml beaker. The dissolved oxygen rate of the material in the 50 ml beaker was then measured. The experiment was performed three times to achieve the results illustrated in Table 3 below.

TABLE 3

| | DO AFTER 0.22 MICRON |
|---|---|
| DO IN SYRINGE | FILTER |
| 42.1 ppm | 39.7 ppm |
| 43.4 ppm | 42.0 ppm |
| 43.5 ppm | 39.5 ppm |

As can be seen, the dissolved oxygen levels that were measured within the syringe and the dissolved oxygen levels within the 50 ml beaker were not significantly changed by passing the diffused material through a 0.22 micron filter, which implies that the microbubbles of dissolved gas within the fluid are not larger than 0.22 microns.

A second test was performed in which a batch of saline solution was enriched with the diffuser of the present invention and a sample of the output solution was collected in an unfiltered state. The dissolved oxygen level of the unfiltered sample was 44.7 ppm. A 0.1 micron filter was used to filter the oxygen-enriched solution from the diffuser of the present invention and two additional samples were taken. For the first sample, the dissolved oxygen level was 43.4 ppm. For the second sample, the dissolved oxygen level was 41.4 ppm. Finally, the filter was removed and a final sample was taken from the unfiltered solution. In this case, the final sample had a dissolved oxygen level of 45.4 ppm. These results were consistent with those in which the Millipore 0.22 micron filter was used. Thus, the majority of the gas bubbles or microbubbles within the saline solution are less than 0.1 microns in size (i.e., less than 100 nanometers in diameter; that is, the majority of the gas bubbles are nanobubbles having an average diameter of less than 100 nanometers).

These results were found to be applicable to ionic aqueous (e.g., water) or saline solutions, and have been confirmed with additional methods (e.g., AFM, nanopipette based experiments).

### Example 2

### Materials and Methods

Reagents:

Neurobasal medium and B27 supplement were purchased from Invitrogen (Carlsbad, Calif.). Other cell culture materials (Hank's balanced salt solution, 0.05% trypsin and antibiotic-antimycotic) were purchased from Mediatech (Washington, D.C.). 5XFAD transgenic mice were purchased from Jackson Laboratory, genotyped and maintained in our animal care facility. Super array kit for analyzing mouse plasticity genes was purchased from SAbiosciences. Primary antibodies, their sources and concentrations used are listed in Table 4. Alexa-fluor antibodies used in immunostaining were obtained from Jackson ImmunoResearch and IR-dye-labeled reagents used for immunoblotting were from Li-Cor Biosciences.

TABLE 4
Antibodies, sources, applications, and dilutions used.

| Antibody | Manufacturer | Catalog# | Host | Application | Dilution/Amount |
|---|---|---|---|---|---|
| NR2A | Cell Signaling | 4205 | Rabbit | WB, ICC/IF | WB 1:500 |
| | | | | | IF 1:100 |
| GLUR1 | Cell Signaling | 8850 | Rabbit | WB, ICC/IF | WB 1:500 |
| | | | | | IF 1:100 |
| β-actin | Abcam | Ab6276 | Mouse | WB | 1:6000 |
| CREB | Cell Signaling | 9197S | Rabbit | WB | 1:500 |
| PSD95 | Abcam | Ab2723 | Mouse | WB, ICC/IF | WB 1:1000 |
| | | | | | IF 1:100 |
| PI3 Kinase | Cell Signaling | 4249S | Rabbit | WB | 1:1000 |
| p110α | | | | | |
| PI3 Kinase | Santa Cruz | sc-7175 | Rabbit | WB | 1:200 |
| p110β | Biotechnology | | | | |
| PI3 Kinase | Santa Cruz | Sc-166365 | Mouse | WB | 1:200 |
| p110γ | Biotechnology | | | | |

WB, Western blot;
ICC, immunocytochemistry;
IHC, immunohistochemistry;
IF, immunofluorescence;
ChIP, chromatin immunoprecipitation.

Animals:

B6SJL-Tg(APPSwFILon,PSEN1*M146L*L286V)6799Vas/J transgenic (5XFAD) mice were purchased from Jackson Laboratories (Bar Harbor, Me.). Male 5XFAD and non-transgenic mice were used for experimentation. Animals were maintained, and experiments were conducted in accordance with National Institutes of Health guidelines and were approved bar the Rush University Medical Center Institutional Animal Care and Use Committee, Antibodies against NR2A (#4205), GluR1 (#8850), and CREB (#9197) were purchased from cell signaling and Arg3.1 antibody was purchased from Abcam (ab23382). Super array kit for analyzing mouse plasticity genes was purchased from SAbiosciences (PAMM-126Z).

Preparation of RNS60:

RNS60 was generated at Revalesio (Tacoma, Wash.) using Taylor-Couette-Poiseuille (TCP) flow as previously described (19, 20). Briefly, sodium chloride (0.9%) for irrigation, USP pH 5.6 (4.5-7.0, Hospira), was processed at 4° C. and a flow rate of 32 mL/s under 1 atm of oxygen back-pressure (7.8 mL/s gas flow rate), while maintaining a rotor speed of 3,450 rpm. Chemically, RNS60 contains water, sodium chloride, 50-60 parts/million oxygen, but no added active pharmaceutical ingredients.

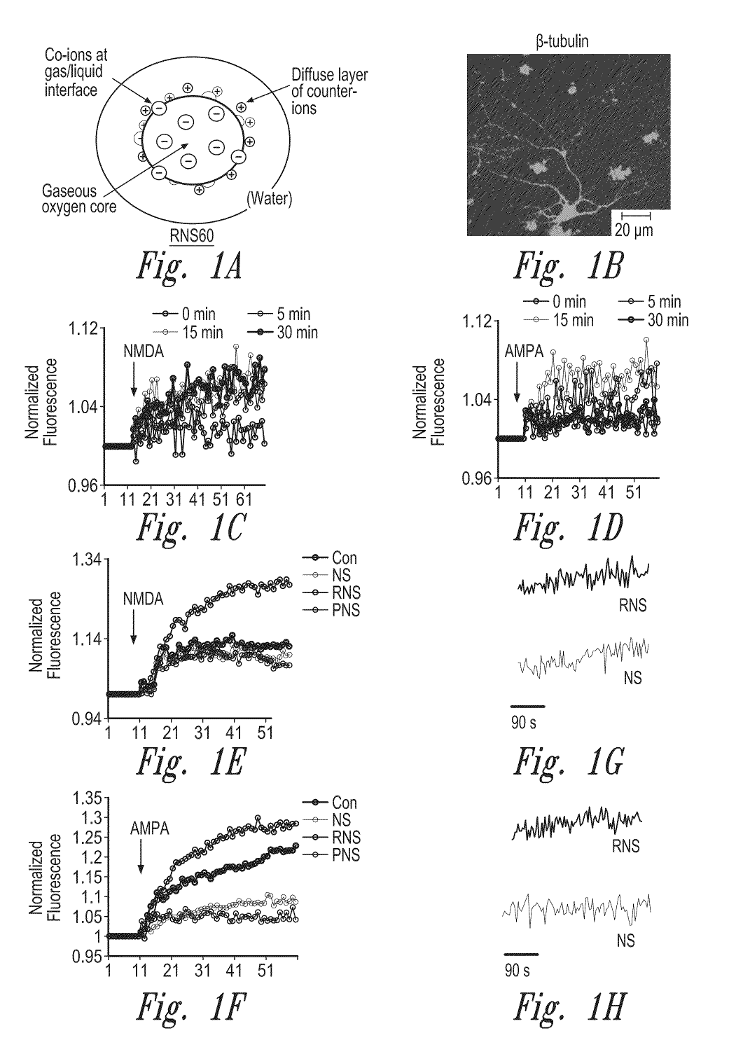

The following controls for RNS60 were also used in this study: a) NS, normal saline from the same manufacturing batch. This saline contacted the same device surfaces as RNS60 and was bottled in the same way and b) PNS60, saline with same oxygen content (55±5 ppm) that was prepared inside of the same device but was not processed with TCP flow. Careful analysis demonstrated that all three fluids were chemically identical (19). Liquid chromatography quadrupole time-of-flight mass spectrometric analysis also showed no difference between RNS60 and other control solutions (19). On the other hand, by using atomic force microscopy, we studied nanobubble nucleation in RNS60 and other saline solutions and observed that RNS60 displays a unique surface nanobubble nucleation profile relative to that of control saline solutions (19). This same relative pattern of nucleation nanobubble number and size was observed when positive potentials were applied to AFM surfaces with the same control solutions, suggesting the involvement of charge in stabilization of nanobubbles in RNS60 (FIG. 1A).

Isolation and Maintenance of Mouse Hippocampal Neurons:

Hippocampal neurons were isolated from fetuses (E18) of pregnant female Ppara null and strain-matched wild-type littermate mice as described by us (21, 22). Briefly, dissection and isolation procedures were performed in an ice-cold, sucrose buffer solution (sucrose 0.32 M, Tris 0.025 M; pH 7.4). The skin and the skull were carefully removed from the brain by scissors followed by peeling off the meninges by a pair of fine tweezers. A fine incision was made in the middle line around the circle of Willis and medial temporal lobe was opened up. Hippocampus was isolated as a thin slice of tissue located near the cortical edge of medial temporal lobe. Hippocampal tissues isolated from all fetal pups (n>10) were combined together and homogenized with 1 ml of Trypsin for 5 min at 37° C. followed by neutralization of trypsin (21, 22). The single cell suspension of hippocampal tissue was plated in the poly-D-lysine pre-coated 75 mm flask. Five minutes after plating, the supernatants were carefully removed and replaced with complete neurobasal media. The next day, 10 μM AraC was added to remove glial contamination in the neuronal culture. The pure cultures of hippocampal neurons were allowed to differentiate fully for 9-10 days before treatment (FIG. 1B).

Measurement of Spine Density and Size:

For counting spine density, E18 hippocampal neurons were stained with Alexa-647 conjugated phalloidin (Cat#A22287) together with MAP2. Only densely stained neurons were selected for the counting. Each cell was magnified at 400× magnification using Olympus BX-51 fluorescence microscope and the total length of the dendrite was measured. The number of spines on all the dendrites counted under oil immersion. As some of the spines were hidden under the dendrite, only those spines that protruded laterally from the shafts of the dendrites into the surrounding area of clear neuropil were selected for the counting. The spine density of a pyramidal neuron was calculated by dividing the total number of spines on a neuron by the total length of its dendrites, and was expressed as the number of spines/10 μM dendrite. The size of the dendritic spines was measured by calculating the ratio of mean fluorescent intensity (MFI) of the spine head and MFI of the dendritic shaft.

Measurement of Axonal Length and the Number of Collaterals:

The length of the primary axon and the number of axonal collaterals were measured by tracing of MAP-2 stained neurons in INKSCAPE™ software tracing tools. All images were scaled under same color intensities. For calculating the number of collaterals, images were magnified at 100× magnification and then the number of collaterals was measured for each 100 μM long axon.

Calcium Influx Assay in Primary Mouse Hippocampal Neurons:

Cultured hippocampal neurons were loaded with Fluo4-fluorescence conjugated calcium buffer (Invitrogen Molecular Probes, Cat# F10471, F10472, F10473) and incubated at 37° C. for 60 mins following manufacture's protocol. After that, fluorescence excitation and emission spectra were recorded in a Perking Elmer Victror X2 Luminescence Spectrometer in the presence of 50 μM of NMDA and 50 μM of AMPA solutions. The recording was performed with 300 repeats at 0.1 ms intervals.

Calcium Influx Assay in Mouse Hippocampal Slices:

Male C57BL/6 animals (n=5) were anesthetized, rapidly perfused with ice cold sterile PBS, decapitated, and finally the whole brain was taken out of the cranium carefully. Dorsoventral slices of the hippocampus were made in TPI PELCO 101 Vibratome series 1000 semi-automatic tissue sectioning system at a thickness of 100 micron. The slice chamber of vibratome machine was filled with cutting solution (sucrose 24.56 g, dextrose 0.9008 g, ascobate 0.0881 g, sodium pyruvate 0.1650 g, and myo-inositol 0.2703 g in 500 mL distilled water) and continuously bubbled with 5% CO₂ and 95% O₂ gas mixture. The whole chamber was kept ice cold during slicing period. Slices were then carefully transferred in Fluo-4 dye containing reaction buffer. The reaction buffer was made prior to the making of brain slices using 10 mL of artificial CSF (119 mM NaCl, 26.2 mM NaHCO₃, 2.5 mM KCl, 1 mM NaH₂PO₄, 1.3 mM MgCl₂, 10 mM glucose, bubbled with 5% CO₂ and 95% O₂ followed by the addition of 2.5 mM CaCl₂) added to one bottle of Fluo-4 dye (Cat# F10471), and 250 mM probenecid. Before transferring slices, a flat bottom 96 well plate (BD Falcon; Cat #323519) was loaded with 50 μL of reaction buffer per well, covered with aluminum foil, and kept in a dark place. Each individual slice was placed in each well loaded with reaction buffer. After transferring slices, the whole plate was re-wrapped with aluminum foil and kept at 37° C. incubator for 20 mins followed by calcium assay in Victor X2 instrument as discussed above.

Immunofluorescence Analysis:

Immunofluorescence analysis was performed as described earlier (23, 24). Briefly, cells cultured in 8-well chamber slides (Lab-Tek II) were fixed with 4% paraformaldehyde for 20 min followed by treatment with cold ethanol (−20° C.) for 5 min and 2 rinses in PBS. Samples were blocked with 3% BSA in PBST for 30 min and incubated in PBST containing 1% BSA and rabbit anti-NR2A (1:100), anti-GluR1 (1:100), anti-PSD95 (1:100) and anti-CREB (1:100). After three washes in PBST (15 min each), slides were further incubated with cy2- and cy5-conjugated secondary antibodies (Jackson ImmunoResearch Laboratories, Inc.). For negative controls, a set of culture slides was incubated under similar conditions without the primary antibodies. The samples were mounted and observed under an Olympus IX81 fluorescent microscope. For tissue staining, brains kept in 4% paraformaldehyde were sectioned in cryostat machine with 30 μm thickness followed by the immunostaining as described before (25).

Cellular Membrane Extraction:

Neuronal membranes were isolated to determine the recruitment of various membrane associated proteins to the membrane. Cells were washed with PBS and scraped in phenol-red-free HBSS to 5 mL ultracentrifuge tubes. The solution was then diluted with 100 mM sodium bicarbonate buffer (pH 11.5) and spun in an ultracentrifuge at 40,000 rpm for 1 hr at 4° C. The resultant supernatant was aspirated and the pellet was immersed in double-distilled H₂0 and SDS and stored at −80° C. overnight. The following day, the pellet was resuspended by repeated grinding and boiling.

Immunoblot Analysis:

Immunoblot analysis was carried out as described earlier (26). Briefly, neuronal cell homogenates were electrophoresed, proteins were transferred onto a nitrocellulose membrane, and protein band was visualized with Odyssey infrared scanner after immunolabeling with primary antibodies followed by infra-red fluorophore-tagged secondary antibody (Invitrogen, Carlsbad, Calif.).

Semi-Quantitative RT-PCR:

Total RNA was isolated from mouse primary hippocampal neurons using Ultra spec-II RNA reagent (Biotecx Laboratories, Inc.) following manufacturer's protocol. To remove any contaminating genomic DNA, total RNA was digested with DNase. Semi quantitative RT-PCR was carried out as described earlier (27) using a RT-PCR kit from Clontech. Briefly, 1 μg of total RNA was reverse-transcribed using oligo(dT)₁₂₋₁₈ as primer and MMLV reverse transcriptase (Clontech) in a 20-μl reaction mixture. The resulting cDNA was appropriately-diluted, and diluted cDNA was amplified using following primers:

nr-2a (mouse):
Sense:
(SEQ ID NO: 1)
5′-GAGGCTGTGGCTCAGATGCTGGATT-3′;
Anti-sense:
(SEQ ID NO: 2)
5′-GGCCCGGCTTGAGGT TTCAGAAAT G-3′;
glur1 (mouse):
Sense:
(SEQ ID NO: 3)
5′-AATGGTGGTACGACAAGGGC-3′;
and
Anti-sense:
(SEQ ID NO: 4)
5′-GGATTGCATGGACTTGGGGA-3′.

Amplified products were electrophoresed on a 1.8% agarose gels and visualized by ethidium bromide staining.

Real-Time PCR Analysis:

It was performed using the ABI-Prism7700 sequence detection system (Applied Biosystems) as described earlier (25, 26) using primers and FAM-labeled probes from Applied Biosystems. The mRNA expressions of respective genes were normalized to the level of GAPDH mRNA. Data were processed by the ABI Sequence Detection System 1.6 software and analyzed by ANOVA.

PCR Super Array Analyses of Plasticity-Associated Genes:

The Mouse Synaptic Plasticity RT² Profiler™ PCR Array (SA Biosciences; Cat #PAMM-126Z) profiles the expression of 84 key genes central to synaptic alterations during learning and memory. Briefly, mouse hippocampal neurons were treated with 10% (v/v) RNS60 and NS for 24 h, followed by isolation of total RNS using Qiagen RNA isolation kit and synthesis of cDNA as described previously (25, 26). Next, cDNA samples were diluted by 100 times and then 2 μl of diluted cCNA was added in each well of 96 well array plate, followed by the amplification of cDNA using SYBR green technology in ABI-Prism7700™ sequence detection system. The resulting Ct value was normalized with housekeeping gene GAPDH and then plotted in heat map explore software.

### Example 3

### RNS60, but Neither NS Nor PNS60, Stimulated Inward Calcium Currents in Cultured Hippocampal Neurons in the Presence of NMDA or AMPA

Inbound calcium currents through NMDA and AMPA receptors have been shown in the art to be associated with the plasticity in hippocampal neurons. In this example, the effect of Applicants' electrokinetically-altered fluid (e.g., RNS60) on calcium influx in cultured mouse hippocampal neurons was determined.

Since the activation of ionotropic glutamate receptors is a very rapid and transient process, calcium influx during short time periods of RNS60 incubation was first measured. Interestingly, no strong induction was observed in either NMDA- (FIG. 1C) or in AMPA- (FIG. 1D) dependent calcium influx after 5, 15, and 30 minutes of incubation with RNS60, even though in all cases, RNS60 showed high amplitude oscillations indicating that the excitability of ionotropic glutamate receptors was not altered.

Next, the effect of RNS60 on NMDA and AMPA-dependent calcium influx was examined in cultured hippocampal neurons after 24 hrs of incubation. Interestingly, RNS60, but neither NS nor PNS60, significantly stimulated calcium influx in the presence of NMDA (FIG. 1E) or AMPA (FIG. 1F). Moreover, prolonged incubation of hippocampal neurons with RNS60 resulted in high frequency calcium influx in the presence of NMDA (FIG. 1G) or AMPA (FIG. 1H) indicating, in particular aspects, that RNS60, but not NS, is a very potent agent in inducing postsynaptic membrane depolarization, which eventually leads to the formation of LTP (20) in hippocampal neurons.

Specifically, FIGS. 1A through 1H show the effect of RNS60, PNS60, and NS on NMDA and AMPA-dependent calcium influx in cultured mouse hippocampal neurons.

Mouse hippocampal neurons were treated with 10% (v/v) RNS60 for 5, 15, and 30 mins under serum free condition followed by treatment with 50 μM NMDA and AMPA as described under materials methods section. (A) Normalized fluorescence value of NMDA-dependent and (B) AMPA-dependent calcium influx monitored for 300 repeats over 90 sec period of time in cultured hippocampal neurons. Next, NMDA-dependent (C) and AMPA-dependent (D) calcium influx in primary neurons after 24 h of RNS60, NS, and PNS60 treatment was analyzed. The result is mean of three independent experiments. Oscillograms of (E) NMDA-driven and (F) AMPA-driven calcium currents in RNS60 and NS-treated primary neuronal cultures. Results are mean±SD of three independent experiments.

### Example 4

### RNS60 was Shown to have an Effect on the Expression of Plasticity-Associated Molecules in Hippocampal Neurons

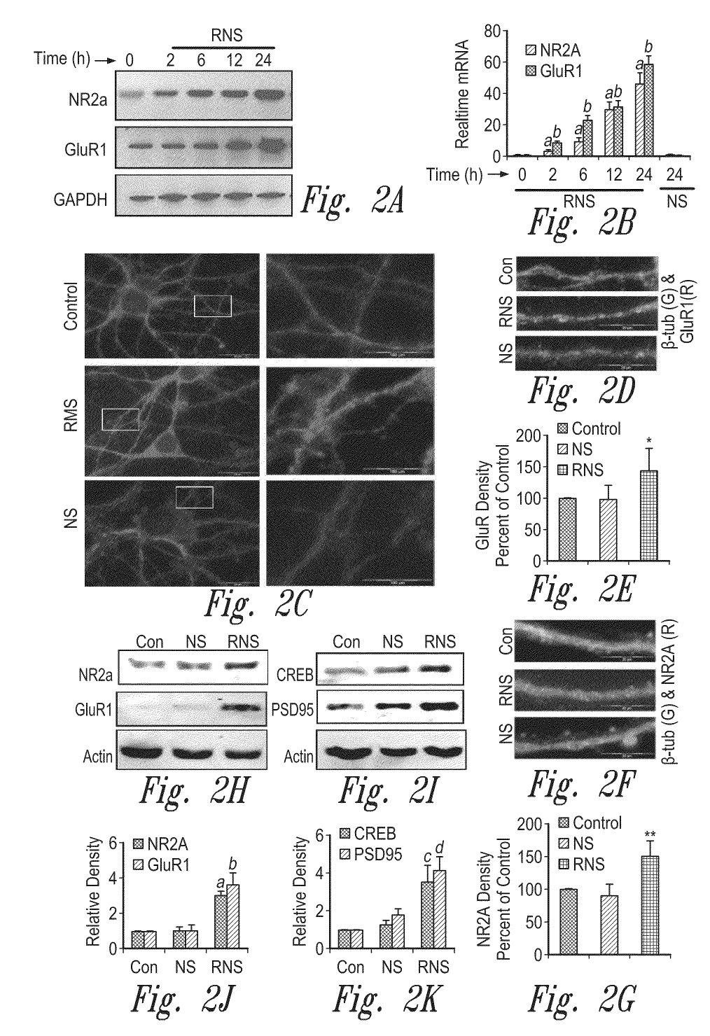

Since RNS60 failed to induce the activation of ionotropic calcium channels in neurons after a short-term incubation, it can be assumed that it is not involved in the transient phosphorylation of NMDA and AMPA receptors subunits. On the other hand, after 24 h of incubation, RNS60 induced NMDA- and AMPA-dependent calcium influx. Therefore, the effect of RNS60 on the expression of plasticity-associated genes in cultured hippocampal neurons was investigated. Time-dependent mRNA analysis shows that RNS60 was capable of increasing NR2A and GluR1 within 2 h of incubation (FIG. 2A-B). However, the level of upregulation of both NR2A and GluR1 increased with time until the duration (24 h) of the study (FIG. 2A-B). These mRNA expression studies were further corroborated with protein expression analysis of NR2A, GluR1, PSD95, and CREB in hippocampal neurons.

Specifically, FIGS. 2A through 2K show the effects of RNS60 in the expression of plasticity-associated proteins in mouse hippocampal neurons. (A) RT-PCR and (B) real-time PCR analyses of NR2A and GluR1 genes were performed in mouse primary hippocampal neurons at 0, 2, 6, 12, and 24 h of RNS60 (10% : v/v) treatment. (C) Immunofluorescene analysis of PSD95 in mouse hippocampal neurons after 24 hrs of RNS60 and NS treatment as described under materials and methods section. Right panels are magnified views of left panel pictures as shown in dotted boxes. (D) Dual immunofluorescence analysis of GluR1 (red) and beta tubulin (green) in mouse primary neurons treated with RNS60 and NS for 24 hrs. (E) Number of GluR1-immunoreactive spines were counted in 50 micron long neuritis of control, NS-, and RNS60-treated hippocampal neurons and then plotted in percent scale compared to control. Results are mean±SD of three independent results. ^{#}p<0.01 vs. control. (F) Double labeling of NR2A (red) and beta tubulin (green) in mouse hippocampal neurons treated with RNS60 and NS for 24 hrs. (G) Number of NR2A-immunoreactive spines was plotted as percent of control in control, NS-, and RNS60-treated neurons. Results are mean±SD of three independent results and ^{##}p<0.001 vs. control. Mouse primary neurons were treated with RNS60 and NS for 24 hrs followed by immunoblot analyses of NR2A and GluR1 (H); CREB and PSD-95 (I). (J and K) Representative histograms are relative densitometric plots of respective immunoblot analyses. ^{a}p<0.01 vs. control NR2A, ^{b}p<0.001 vs. control GluR1, ^{c}p<0.01 vs. control CREB, and ^{d}p<0.01 vs. control PSD95. Results are mean±SD of three independent experiments.

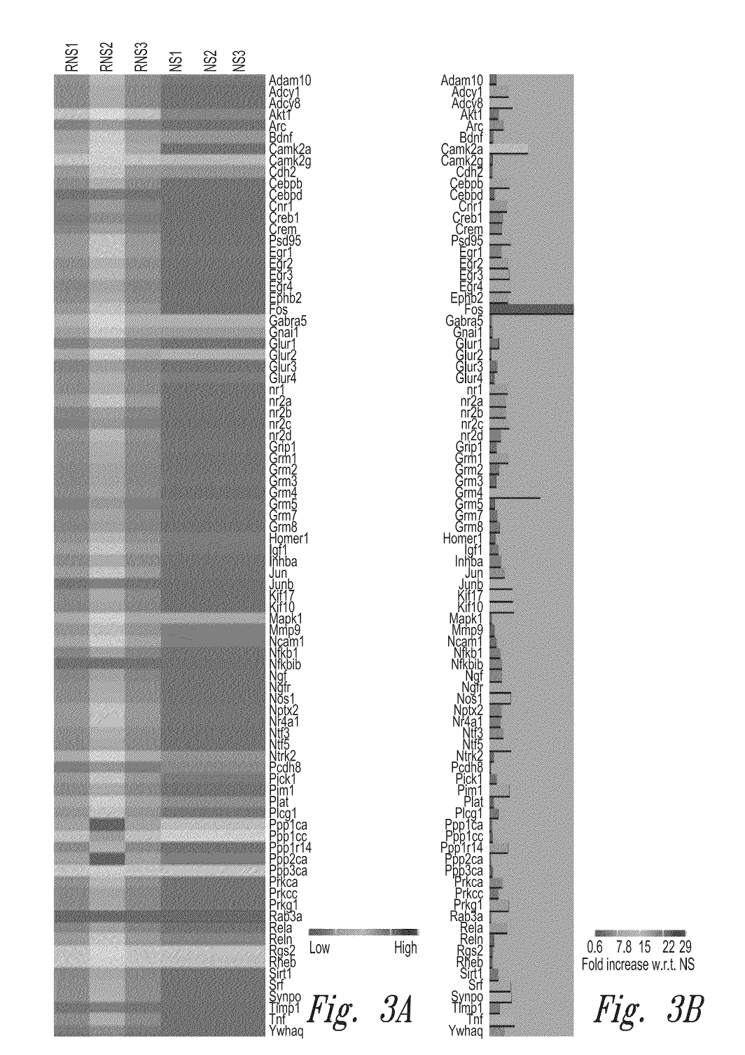

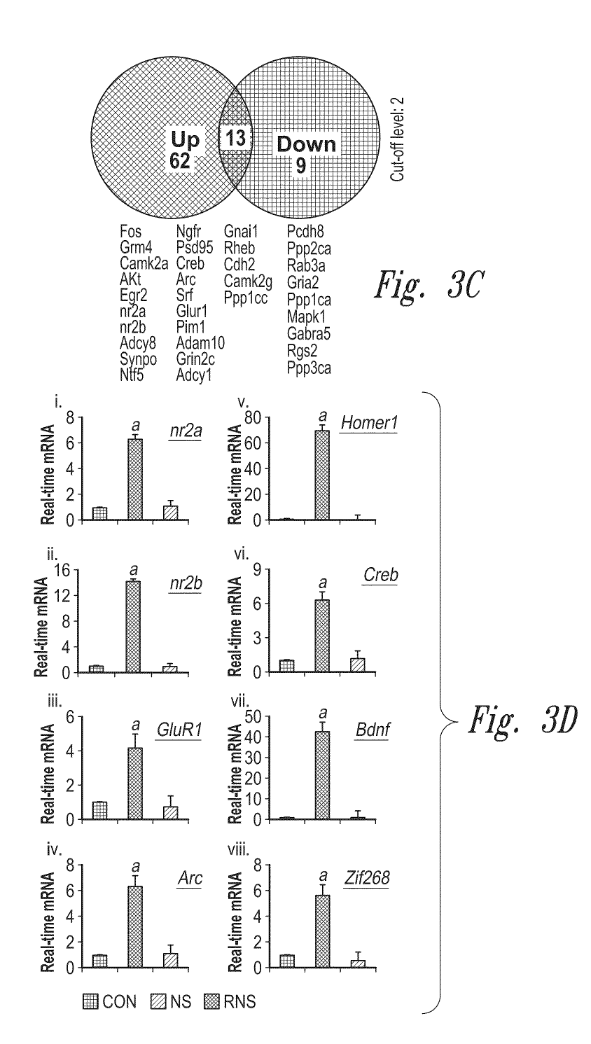

First, immunofluorescence analysis of PSD95 (FIG. 3C), GluR1 (FIG. 3D-E), and NR2A (FIG. 3F-G) was performed. RNS60 strongly upregulated the protein expression of PSD95, NR2A, and GluR1 in the projections of hippocampal neurons (FIG. 3C-G). Immunoblot analyses of NR2A and GluR1 (FIG. 3H-I) along with CREB and PSD95 (FIG. 3J-K) further confirmed that RNS60 significantly stimulated the expression of plasticity-related proteins in hippocampal neurons. These results were specific as NS had no effect on the expression of these plasticity-related proteins.

Plasticity is controlled by multiple proteins. Therefore, the question of whether RNS60 regulated only NR2A and GluR1 or other plasticity-associated hippocampal molecules are also controlled by RNS60 was examined. An mRNA-based super array analysis of plasticity-related genes in both RNS60- and NS-treated cultured hippocampal neurons was performed, and the results summarized in a heat-map presentation (FIG. 3A-B). Strikingly, 62 of 84 analyzed genes were upregulated, 9 genes were down-regulated, and 13 genes remained unaltered in RNS60-treated hippocampal neurons as compared to NS-treatment (FIG. 3C). Among the upregulated genes observed were: IEGs including arc, zif-268, and c-fos; synapse-associated genes including synpo, adam-10, and psd-95; and most interestingly genes encoding NMDA receptor subunits including nr1, nr2a, nr2b, and nr2c; genes of AMPA receptor subunit glur1; and genes for neurotrophic factors and their receptors including bdnf, nt3, nt5, and ntrk2. Furthermore, CREB is an important molecule for plasticity as it controls the transcription of various plasticity-related molecules (29, 30). It is interesting to see that RNS60 upregulates CREB as well as different signaling molecules that are involved in the activation of CREB. For example, the adenylate cyclase pathway is known to activate CREB via the cAMP-protein kinase A (PKA) pathway (31). RNS60 treatment increases the expression of genes encoding for different adenylate cyclases (adcy1 and adcy8) in mouse hippocampal neurons as compared to NS treatment (FIG. 3A-B). CREB is also activated by Ca²⁺/calmodulin-dependent protein kinase II (CAM kinase II) and Akt (31, 32). Accordingly, RNS60 also upregulated the expression of camk2a and akt1 (FIG. 3A-B). In contrast to the upregulation of plasticity-associated molecules, RNS60 treatment down-regulated the expression of Gria2, Ppp1ca, Ppp2ca, and Ppp3ca, proteins encoded by which genes are known to support long-term depression (FIG. 3A-C).

In order to validate some of the array-based mRNA results, quantitative real-time PCR analysis of eight randomly chosen genes from the list was performed, confirming that RNS60 indeed upregulated the mRNA expression of nr2a (FIG. 3Di), nr2b (FIG. 3Dii), glur1 (FIG. 3Diii), arc (FIG. 3Div), homer-1 (FIG. 3Dv), creb (FIG. 3Dvi), bdnf (FIG. 3Dvii), and zif-268 (FIG. 3Dviii) by several folds in hippocampal neurons as compared to untreated neurons. These results were RNS60-specific, as NS-treatment did not upregulate the expression of these genes (FIG. 3D).

Specifically, FIGS. 3A through 3Dviii show the effects of RNS60 on the expression of plasticity-associated genes in cultured mouse hippocampal neurons. Mouse primary neurons were treated with 10% RNS60 and NS for 24 h followed by the analyses of plasticity-associated gene expression from total mRNA by mRNA-based super array technology. (A) Heatmap expression profile of 84 plasticity-associated genes as derived from mRNA-based array. Red represents the minimum and blue represents the maximum level of expression. (B) The histogram summary of expression of all representative genes shown in the heatmap. (C) Venn diagram summarizes the list of genes upregulated, downregulated, and unaltered in RNS-treated water. (D) Realtime mRNA analyses of randomly selected eight different genes including NR2A (i), NR2B (ii), GluR1 (iii), Arc (iv), Homer1 (v), CREB (vi), BDNF (vii), and Zif-268 (viii) in RNS and NS-treated mouse hippocampal neurons under similar condition. Results are mean±SD of three independent experiments. ^{a}p<0.001 vs. control.

According to particular aspects of the present invention, therefore, taken together, these results indicate and confirm that RNS60 stimulates the expression of plasticity-associated proteins in hippocampal neuronal cultures.

### Example 5

### RNS60 Upregulated Plasticity-Associated Molecules and Stimulated Calcium Influx in Primary Mouse Hippocampal Neurons Via Phosphatidylinositol 3-Kinase (PI3K)

In this Example, mechanisms by which RNS60 increases plasticity in cultured hippocampal neurons was examined.

Applicants have observed that RNS60 activates PI3K in microglial cells (19). Because PI3K is linked to a diverse group of cellular functions, in this Example, the question of whether PI3K was involved in RNS60-mediated stimulation of plasticity in hippocampal neurons was examined. At first, the effect of RNS60 on PI3K activation in hippocampal neurons was tested. Class IA PI3K, which is regulated by receptor tyrosine kinases, consists of a heterodimer of a regulatory 85-kDa subunit and a catalytic 110-kDa subunit (p85:p110α/β/δ). Class IB PI3K, on the other hand, consists of a dimer of a 101-kDa regulatory subunit and a p110γ catalytic subunit (p101/p110γ). While in resting condition, subunits of PI3K are located mainly in cytoplasm, upon activation, these are translocated to the plasma membrane (33, 34). Therefore, the activation of class IA and IB PI3K by the recruitment of p110α, p110β and p110γ to the plasma membrane was examined.

Results.

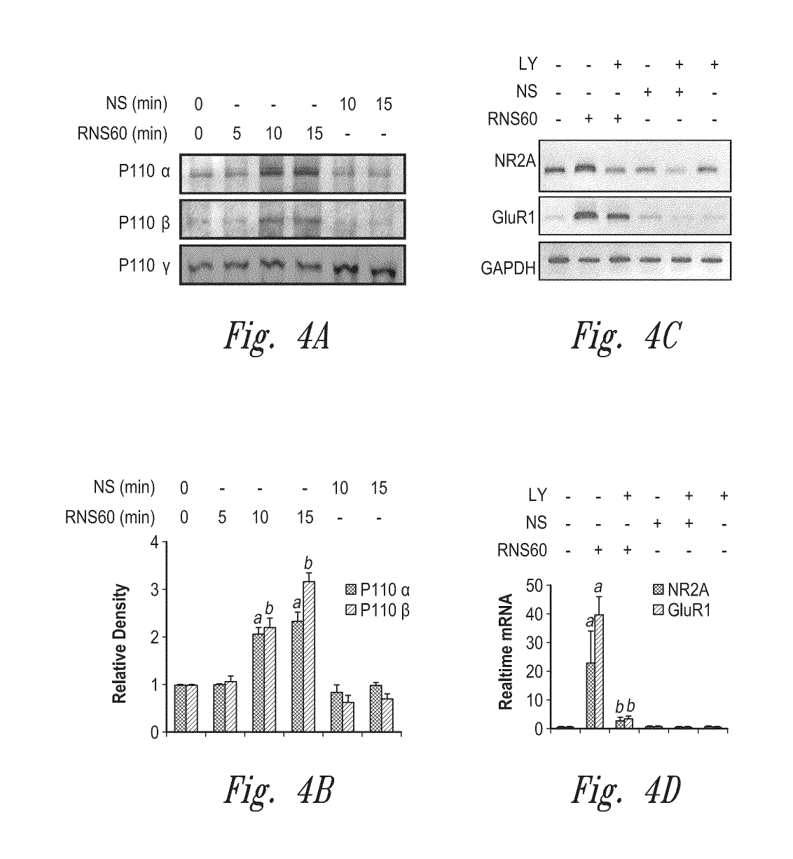

Western blotting of membrane fractions for p110 subunits suggests that RNS60 specifically induces the recruitment of p110α and p110β, but not p110γ, to the plasma membrane (FIG. 4A). Densitometric analysis of the p110α and p110β at different time points of RNS60 stimulation indicates significant activation of PI3K at 10 and 15 min (FIG. 4B). On the other hand, no activation of p110α and p110β PI3K at 5 min of RNS60 stimulation (FIG. 4A-B) was observed. Again these results were specific as NS remained unable to activate p110α and p110β PI3K at either 10 or 15 min of RNS60 stimulation. Together, these results suggest that RNS60 activates type IA PI3K p110α and p110β, but not type IB PI3K p110γ, in hippocampal neurons.

Next, to understand whether modulation of PI3K signaling pathway is involved in the RNS60-induced neuronal plasticity, primary mouse hippocampal neurons were pretreated with 2 μM PI3K inhibitor (LY294002) for 15 min, followed by stimulation with 10% RNS60 or NS. After 3 h of stimulation, mRNA expression of NR2A and GluR1 was monitored by RT-PCR and real-time PCR. In this instance as well, RNS60 treatment increased the expression of NR2A and GluR1 (FIG. 4C-D). However, LY294002 abrogated RNS60-mediated increase in NR2A and GluR1 expression in hippocampal neurons (FIG. 4C-D).

Specifically, FIGS. 4A through 4D show the role of PI3K pathway in RNS60-mediated upregulation of plasticity-associated genes in mouse hippocampal neurons. (A) Mouse hippocampal neurons were stimulated with RNS60 and NS for 5, 10, 15, and 30 minutes under serum-free condition followed by the immunoblot analyses of p110α, β, and γ in membrane fractions. (B) Relative densitometric analyses of p110α and β immunoblot in same treatment condition. Results are mean±SD of three independent experiments. ^{a}p<0.001 vs control p110; ^{b}p<0.001 vs control-p110. Cells pretreated with 2 μM LY294002 for 15 min were stimulated with 10% RNS60. After 3 h of stimulation, the mRNA expression of NR2A and GluR1 was analyzed by semi-quantitative RT-PCR (C) and real-time PCR (D). Results are mean±SD of three independent experiments. ^{a}p<0.001 vs control; ^{b}p<0.001 vs RNS60.

However, LY29402 inhibits the activation of both class 1A and 1B PI3K. Therefore, our next aim was to identify the specific class of PI3K that was involved in the RNS60-mediated upregulation of NR2A and GluR1 in hippocampal neurons. We used three different PI3K inhibitors: GDC-0941 (an inhibitor of p110α); TGX-221 (an inhibitor of p110β); and AS-605240 (an inhibitor of p110γ). Interestingly, the pretreatment of α and β suppressed the RNS60-stimulated expression of NR2A and GluR1 in cultured hippocampal neurons indicating that class 1A, not class 1B PI3K, is involved in the upregulation of plasticity-associated genes in RNS60-stimulated neurons.

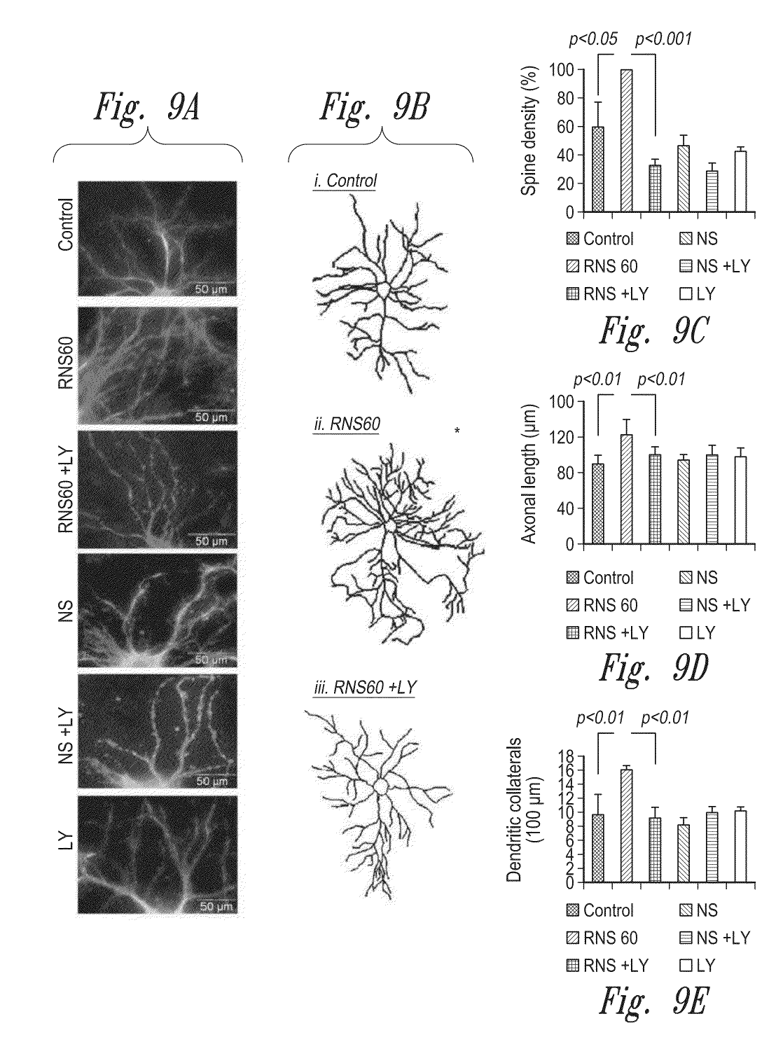

Since the reduced expression of NR2A and GluR1 is linked to the decreased spine density and axonal maturation of neurons, the role of PI3K pathway in RNS60-mediated increase in spine density and axonal morphologies in cultured hippocampal neurons was studied. Applicants observed that 15 mins. pretreatment with 2 μM LY29402 prior to RNS60 treatment significantly decreased the spine density in RNS60-treated, but not in NS-treated, hippocampal neurons (FIG. 9A). The effect was further quantified by counting spine density (FIG. 9C). Next, the effect of LY29402 on the axonal length and number of collaterals in RNS60-treated neurons was examined. Interestingly, LY29402 significantly attenuated the length of primary axon and number of collaterals in RNS60-treated neurons (FIG. 9Bi-iii), which was further quantified as shown in FIG. 9D-E.

Specifically, FIGS. 9A, 9B(i)-9B(iii) and 9C-9E show activation of PI3K regulates morphological plasticity in RNS60-treated mouse hippocampal neurons. (A) LY294002 pre-treated mouse hippocampal neurons were stimulated with RNS60 and NS for 48 hrs followed by double-immunostaining with MAP2 (green) and Phalloidin (red) to demonstrate the spine density. (B) Neurons were traced by Inkscape software after 48 hrs. of treatment with RNS and NS. (C) Spine density, axonal length, and dendritic branches were measured from 10 different neurons of each treatment group. *p<0.05 vs. control and **p<0.01 w.r. to spine density RNS60-treated neurons.

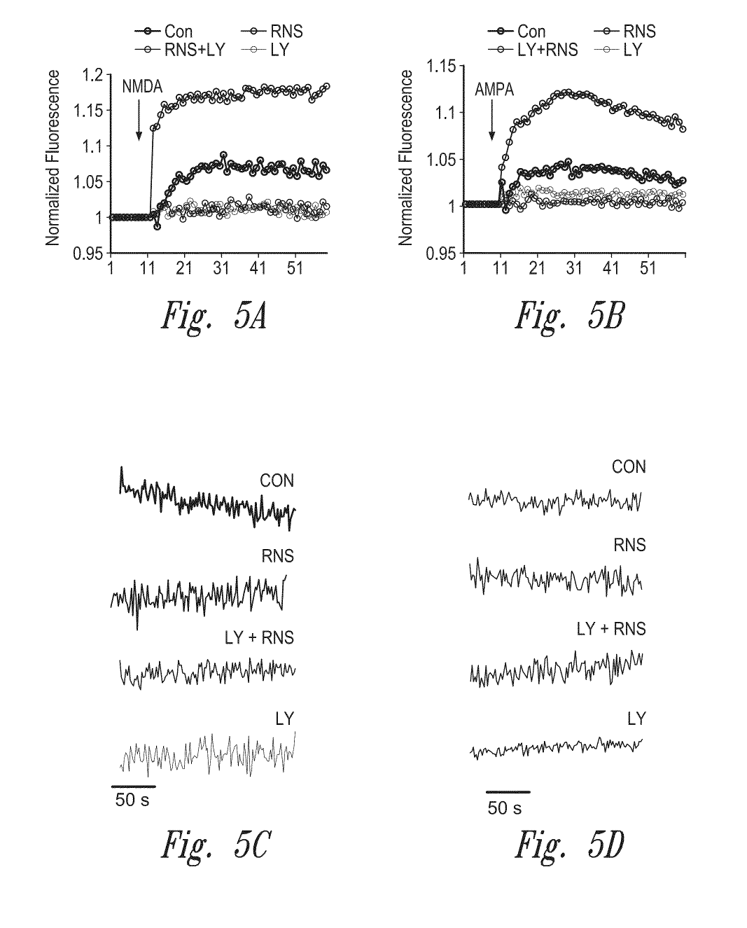

The critical event leading to the induction of long-term potentiation appears to be the influx of calcium ions into the postsynaptic spine. Therefore, the effect of LY294002 on RNS60-induced calcium influx was next examined. As shown above, RNS60 treatment stimulated calcium influx in the presence of either NMDA (FIG. 5A-B) or AMPA (FIG. 5C-D). However, LY294002 abated the stimulatory effect of RNS60 on NMDA- (FIG. 5A-B) and AMPA-induced (FIG. 5C-D) calcium influx.

Specifically, FIGS. 5A through 5D show that activation of PI3K regulates both NMDA- and AMPA-sensitive calcium influx in RNS60-treated mouse hippocampal neurons. Mouse hippocampal neurons pre-treated with 2 μM LY294002 for 15 mins were incubated with 10% (v/v) RNS60 for 24 h under serum free condition followed by the measurement of calcium influx in the presence of 50 μM NMDA (A) and AMPA (B). Representative images are (C) NMDA- and (D) AMPA-mediated oscillograms of calcium influx in control, RNS60-, (RNS60+LY)-, and LY-treated primary hippocampal neurons. Results are mean of three independent experiments.

According to particular aspects of the present invention, therefore, taken together, these results indicate and confirm that RNS60 stimulates plasticity in hippocampal neurons through the activation of the PI3K pathway.

### Example 6

### RNS60 Treatment Increased the Expression of Plasticity-Associated Proteins In Vivo in the Hippocampus of 5XFAD Transgenic Mice

In this Example, the effect of RNS60 treatment on the expression of these hippocampal proteins in 5XFAD mice, an accelerated model of AD, was investigated.

Strong down regulation of NMDA and AMPA receptor proteins and loss of calcium excitability in hippocampal neurons are often observed in AD brain. According to particular aspects, Applicants conceived that reversal of these cellular events may have implications for hippocampal plasticity and hippocampal-dependent learning and memory. Therefore, the effect of RNS60 treatment on the expression of these hippocampal proteins in 5XFAD mice, an accelerated model of AD was investigated.

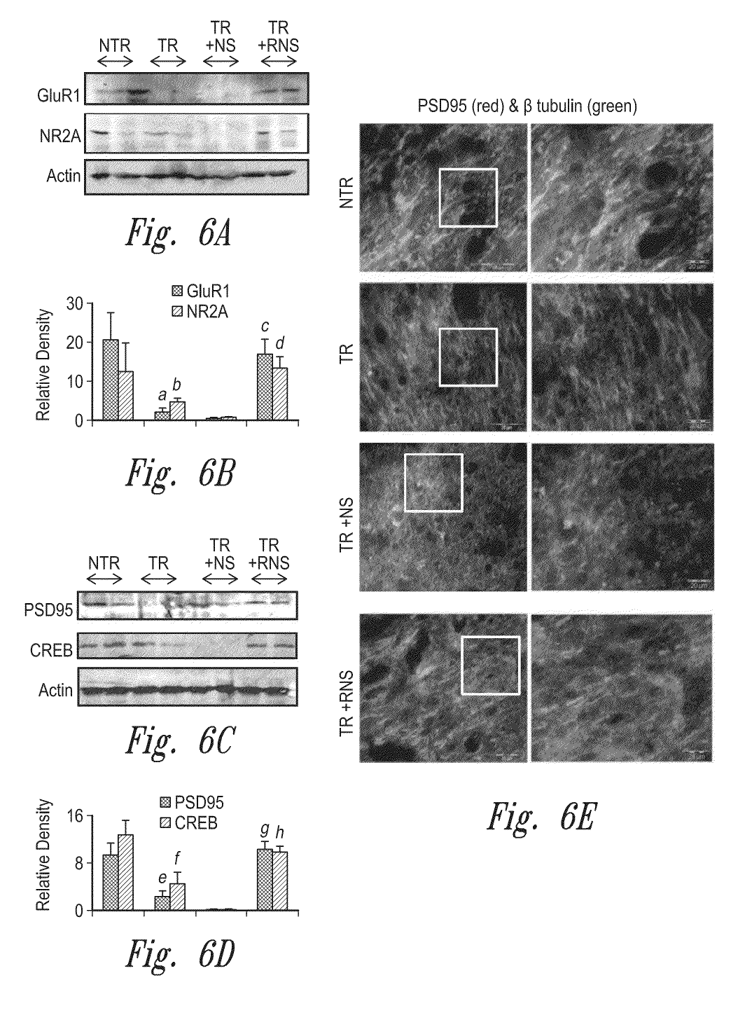

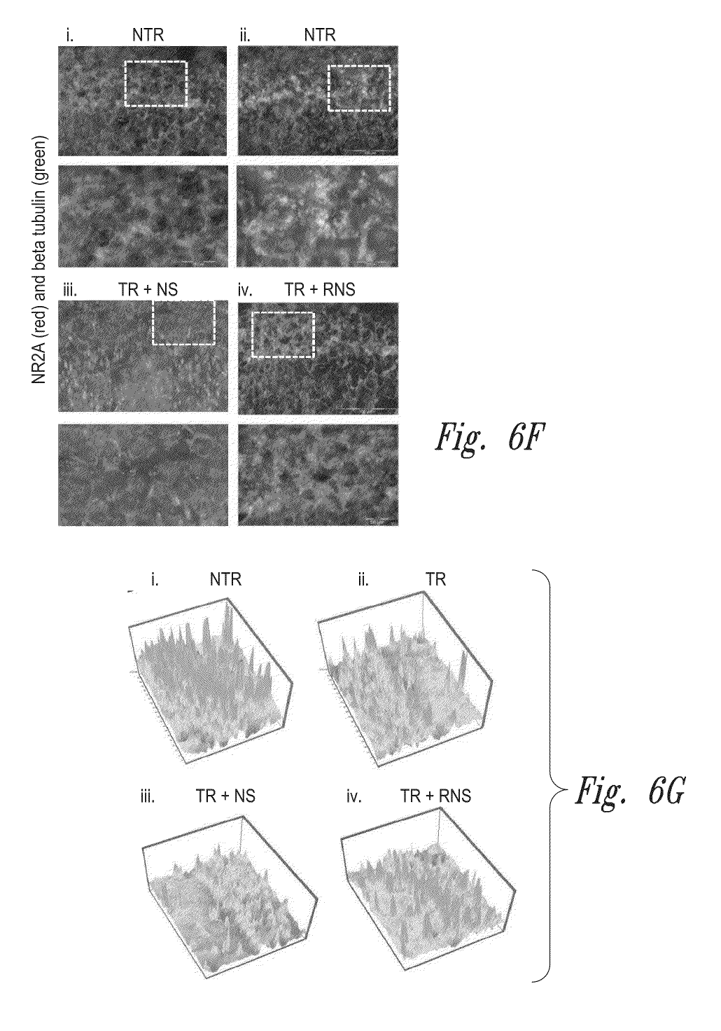

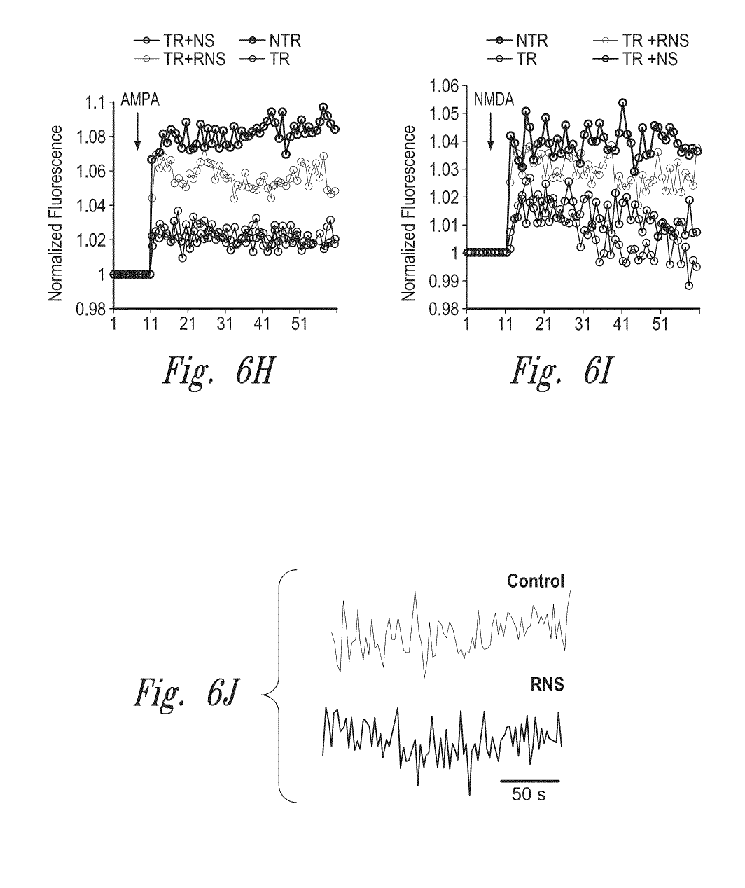

Immunoblot analyses of different hippocampal proteins in 5XFAD transgenic (TR) and age-matched non-transgenic (NTR) mice, as well as in transgenic animals treated with RNS60 (TR+RNS60) or NS (TR+NS) was first performed. Immunoblot analysis revealed a strong down-regulation of ionotropic glutamate receptor subunits including NR2A and GluR1 (FIG. 6A-B), and other plasticity-associated proteins including PSD-95 and CREB (FIG. 6C-D), in the hippocampus of TR mice as compared to NTR mice. This deficit was almost completely restored by the treatment with RNS60, while NS remain ineffective. Consistently, immunofluorescence analysis showed that RNS60 treatment significantly upregulated the expression of PSD95 (FIG. 6E) and NR2A (FIG. 6Fi-iv) in the hippocampus of TR animals. Of note, the number of signal hotspots in representative 3D intensity plot of RNS60-treated TR mice was similar to that of NTR mice (FIG. 6Gi-iv).

The question of whether, if similar to cultured neurons, calcium influx in hippocampal slices of adult mice could be recorded. Consistent with decreased expression of plasticity-associated molecules in hippocampus of TR mice as compared to NTR mice, AMPA- (FIG. 6H) and NMDA-dependent (FIG. 6I) calcium influx was less in hippocampal slices of TR mice as compared to NTR mice. However, AMPA- and NMDA-dependent calcium influx increased in hippocampal slices of TR mice after RNS60 treatment (FIG. 6H-I). Interestingly, the level of calcium influx in hippocampal slices of (TR+RNS) group was very much similar to those observed in hippocampal slices of the NTR group. As evident from FIG. 6J, RNS60 treatment evoked oscillatory amplitude in the hippocampus of TR mice to a level that is similar to untreated NTR mice.

Specifically, FIGS. 6A through 5J show the effect of RNS60 on the expression of plasticity-associated molecules in vivo in the hippocampus of 5XFAD transgenic animals. Five month old transgenic mice (n=5 per group) were injected i.p. with RNS60 and NS (300 μL/mouse/2 d) for 60 days. After that, animals were sacrificed and their hippocampi were analyzed for the expression of different plasticity-associated proteins. Immunoblot analyses of NR2A and GluR1 (A); PSD-95 and CREB (C) in the hippocampal extracts of NTR (non-transgenic), TR (transgenic), TR+RNS, and TR+NS animals. Relative densitometric analyses of GluR1 and NR2A (B) & PSD95 and CREB (D). Results are mean±SEM of five mice per group. ^{a}p<0.001 vs. control-GluR1; ^{b}p<0.005 vs. control-NR2A; ^{c}p<0.001 vs. TR-GluR1; ^{d}p<0.005 vs. TR-NR2A; ^{e}p<0.005 vs. control-PSD95; ^{f}p<0.001 vs. control-CREB; ^{g}p<0.001 vs. TR-PSD95; ^{h}p<0.005 vs. TR-CREB. (E) Hippocampi of NTR and TR animals fed with RNS60 and NS were stained with PSD95 (red) and beta-tubulin (green). Representative images showed the distribution of PSD95 in the presynaptic branches of CA1 nucleus. Right side panels are the magnified presentations of left side images boxed under dotted white line. (F) Double labeling of NR2A (red) and beta tubulin (green) in CA-1 hippocampus of NTR-(i) and TR-(ii) animals fed with RNS60-(iii) and NS-(iv). Bottom panels are magnified views of top panel images highlighted in dotted squares. (Gi-iv) The distribution of NR2A in the CA-1 nucleus was shown in a 3D contour diagram as signal hotspot in Image Dig software. Red, yellow, green, and blue colors indicate the region with less, moderate, high, and very high distribution of NR2A receptors respectively. (H) AMPA- and (I) NMDA-dependent calcium currents were measured in the hippocampal slices of NTR, TR, (TR+RNS60), and (TR+NS) animals as described under materials and methods. (J) Representative oscillograms of calcium currents in NTR and (TR+RNS60)-fed hippocampal slices.

### Example 7

### RNS60, but Neither NS, PNS60 Nor RNS 10.3, Induced Morphological Plasticity in Cultured Hippocampal Neurons

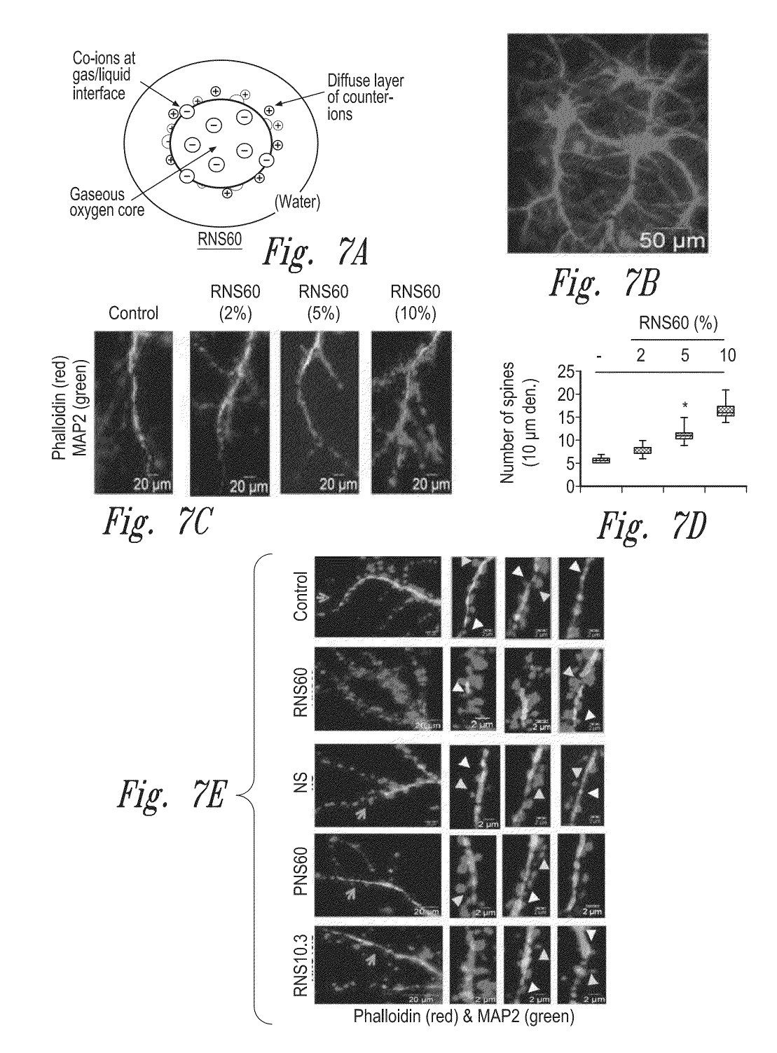

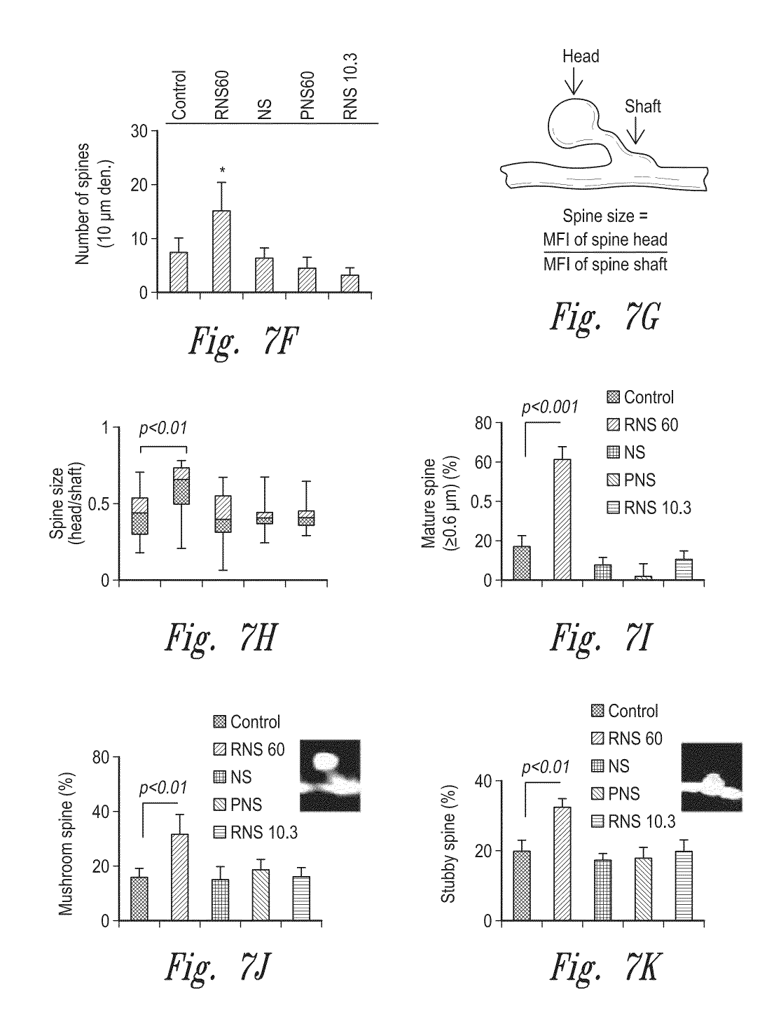

Since the formation and maturation of dendritic spines contribute directly to the long-term enhancement of synaptic efficacy of hippocampal neurons underlying the formation of learning and memory, the effect of RNS60 on the number, size, and maturation of dendritic spines was studied. First, the effect of 2%, 5% and 10% v/v RNS60 on the spine density was analyzed. Interestingly, RNS60 dose-dependently increased the density of dendritic spines in cultured hippocampal neurons (FIG. 7C-D). A detailed morphological analyses further revealed that RNS60, but not other salines such as NS, PNS, and RNS 10.3 (Solas), stimulated the number (FIG. 7E-F), size (FIG. 7G-H), and maturation (FIG. 7J-K) of dendritic spines in hippocampal neurons, indicating that RNS60 enhances the synaptic maturation of hippocampal neurons by enriching the density and size of dendritic spines.

Specifically, FIGS. 7A through 7K show the effect of RNS60, NS, PNS60, and RNS10.3 on the number, size, and maturation of dendritic spines in hippocampal neurons. A) Schematic representation of RNS60. Three weeks old hippocampal neuronal cultures (B) were treated with 2, 5, and 10% RNS60 for two days followed by the immunostaining with neuronal marker MAP2 (green) and Alexa-647 conjugated phalloidin (red) for spines (C). Boxplot analyses for quantifying the spine density in neurons by different doses of RNS60 (D). Control-, RNS60-, NS-, PNS60-, and RNS10.3-treated neurons were double-stained with MAP2 and Phalloidin after 48 h of incubation (E). Left side images are the larger view of dendrites and three right side images per group show the spine density of dendrites collected from three separate images from each group. The spine density (F) was measured from Phalloidin-stained neurons and plotted as a function of 10 μm long dendrites (G). The cartoon shows the strategy applied to measure the spine size. (H) Accordingly, spine size was calculated from 20 images of dendrites. (I) Spines with head to neck ratio of 0.6 were considered as matured spines and their number was counted and plotted. Number of mushroom (J) and stubby (K) spines were counted from 10 different images and plotted for control-, RNS60-, NS-, RNS10.3-, and PNS60-treated hippocampal neurons.

Different morphological changes in the axon of a pre-synaptic neuron including the length of primary axons, number of collaterals, and number of tertiary branches are also associated with the long-term synaptic facilitation (Hatada, et al., J. Neurosci 20, RC82).

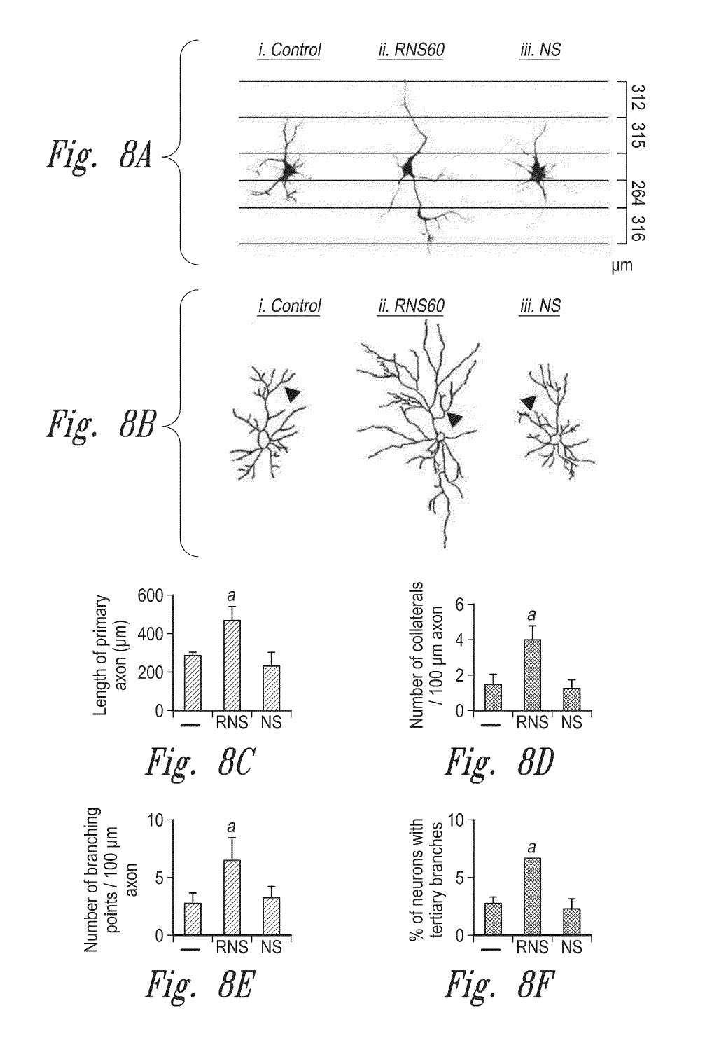

Therefore, the effect of RNS60 on the enlargement of primary axon, the formation of new collaterals, and the number of neurons with tertiary branches was analyzed. Interestingly, the tracing analyses (n=10 per group) clearly indicated that RNS60, but not NS, significantly stimulated the elongation of primary axons (FIGS. 8A and 8C), the number of collaterals (FIGS. 8B & 8D), and the number of neurons with tertiary branches (FIG. 8E-F), demonstrating that RNS60 stimulates the growth of axons, which in turn is related to the increased synaptic activity.

Specifically, FIGS. 8A through 8F show that RNS60 stimulates the length, and collaterals of primary axon in cultured hippocampal neurons. (A) Hippocampal neuronal cultures were treated with 10% RNS60 and NS for two days followed by the immunostaining with neuronal marker MAP2. After that neurons were traced in scalable vector graphics (SVG) software INKSCAPE™ for only primary axon (A) and for detailed branching (B). (C) The length of primary axon, Number of (D) collaterals per 100 μm axon, (E) branching points, and (F) tertiary branches (plotted in a percent scale to RNS60) were calculated from twenty images of each treatment group. ^{a}p<0.01 vs. control.

According to particular aspects of the present invention, therefore, taken together, these results indicate and confirm that RNS60 stimulates plasticity in hippocampal neurons in vivo, enhances the synaptic maturation of hippocampal neurons by enriching the density and size of dendritic spines, and enhances the length of primary axons, number of collaterals, and number of tertiary branches.

### Example 8

### Squid Giant Synapse Preparation, Solutions and Methods

All experiments were carried out at the Marine Biological Laboratory in Woods Hole, Mass. (MBL). As in previous research with this junction (Katz and Miledi 1967, 71, Llinas et al., 1976, 1981, Augustine and Charlton, 1986) one squid (Loligo paelli) stellate ganglion was rapidly removed from the mantle following decapitation and the stellate ganglion was dissected from the inner surface of the mantle under running seawater. Following isolation, the ganglion was placed in a recording chamber and submerged in artificial seawater (ASW). The ganglion was set in the chamber such that both the presynaptic and postsynaptic terminals could be directly visualized for microelectrode penetration. A total of 70 synapses were studied with the number of dissected preparation being close to one hundred fifty; some synapses dissected were not usable as clear anatomical and optimal transparency is required for experimental implementation stability.

RNS60.

RNS60 is a physically modified normal saline (0.9%) solution generated by using a rotor/stator device, which incorporates controlled turbulence and Taylor-Couette-Poiseuille (TCP) flow under high oxygen pressure (see Applicants U.S. Pat. Nos. 7,832,920, 7,919,534, 8,410,182, 8,445,546, 8,449,172, and 8,470,893, all incorporated herein by reference in their entireties for their teachings encompassing Applicants' device, methods for making the fluids, and the fluids per se). Briefly, for producing the RNS60 used in the working examples disclosed herein, sodium chloride (0.9%), USP pH 5.6 (4.5-7.0, Hospira), is processed using Applicants' patented device at 4° C. with a flow rate of 32 mL/s under 1 atm of oxygen backpressure (7.8 mL/s gas flow rate) while maintaining a rotor speed of 3,450 rpm. These conditions generate a strong shear layer at the interface between the vapor and liquid phases near the rotor cavities, which correlates with the generation of small bubbles from cavitation, shearing and other forces. The resulting fluid is immediately placed into glass bottles (KG-33 borosilicate glass, Kimble-Chase) and sealed using gray chlorobutyl rubber stoppers (USP class 6, West Pharmaceuticals) to maintain pressure and minimize leachables. When tested after 24 h, the oxygen content was 55±5 ppm (ambient temperature and pressure). Chemically, RNS60 contains water, sodium chloride, 50-60 parts/million oxygen, but no active pharmaceutical ingredients. The structure and activity of the fluids is stable for at least months or at least years at 4° C. in the closed containers at atmospheric pressure.

Superfusion Solutions.

Two standard and one physically modified artificial seawater (ASW) solutions were used in these experiments. Salts were added to 1 liter of distilled water or a 40 ml bottle of physically modified water such that the final salt composition and pH were identical in every case (423 mM NaCl, KCl 8.27 mM, CaCl2 10 mM, MgCl2 50 mM, buffered to 7.2 with HEPES, salinity 3.121%). ASW made with distilled water or physically modified saline was prepared each day and keep at 4° until the start of the experiment. At the start of an experiment, the control ASW and one 40 ml bottle of RNS60 ASW was removed from the refrigerator, brought to room temperature, and the oxygen content measured. Several synapses (5-15) were dissected and studied each day. All experiments were carried out at room temperature (15-18° C.) as is our standard practice.

The physically modified saline was RNS60 ASW, made using RNS60 that contains oxygenated nanobubbles prepared with TCP flow. The standard ASWs were: 1) Control ASW, made using distilled H2O with air diffusion oxygenation (without bubbling); and 2) NS30612 ASW made using unprocessed normal saline from the same source solution as used to make RNS60. RNS60 and NS30612 were a gift from Revalesio. Removal of the synapse from the squid was carried out under running seawater. All procedures before beginning the recording sessions, the fine dissection and synapse impalement, were carried out using standard ASW because of the large volume of ASW required. In our initial experiments synaptic transmission in NS30612 was found to be indistinguishable from that recorded in our standard control ASW (not shown); ASW was used as the initial step in all experiments.

Oxygen Content Measurement.

Oxygen measurement of each superperfusate was determined using a Unisense MicroOptode near infrared (NIR, 760-790 nm) sensing probe (400 μm) corrected for temperature and salinity. The mean and s.e.m. of the oxygen content of each of the ASWs measured over 10 min were: 1) Control ASW 268±0.26 μmol/l (8.57 ppm) 2) RNS60 ASW 878±0.8 μmol/l (28.1 ppm); 3) Normal Saline (NS) 266±0.18 μmol/l (8.5 ppm). The oxygen content of RNS60 ASW is quite stable. Over the period of a typical experiment, about 30 min, oxygen content of the RNS60 ASW decreased by about 8.7%.

General Electrophysiology.

Following stable presynaptic and postsynaptic microelectrode impalement and the demonstration of synaptic transmission following presynaptic electrical stimulation the experimental procedure was initiated. The postsynaptic electrodes were beveled to reduce their resistance (<1 MΩ) and thus improved the signal/noise ratio. To evaluate changes in the RC properties of the postsynaptic membrane, the decay constant of the falling phase of the EPSPs was estimated using a built in curve fit function for a decaying exponential (exp Xoffset, Igor Pro, Wavemetrics, Inc).

Evoked Synaptic Transmission.

Single glass microelectrodes were inserted into the largest (most distal) presynaptic terminal and the corresponding postsynaptic axon. Evoked presynaptic and postsynaptic action potentials were recorded following a standard protocol (Llinas R. et al 1981). The synapse was activated either by extracellular electrical stimulation of the presynaptic axon via an insulated silver wire electrode pair or by direct depolarizing the presynaptic terminal through an intracellular electrode. Nerve stimulation was delivered as single stimulus or a train (250 ms at 200 Hz delivered at 1 Hz).

Spontaneous Release as Determined by Fourier Analysis of Postsynaptic Noise Level.

Spontaneous transmitter release was recorded postsynaptically as noise fluctuation of the postsynaptic membrane potential at the synaptic junction (Lin et al., 1990). Synaptic noise measurements provided a second method to assess synaptic viability, and a probe to understand possible effects of RNS60 on spontaneous synaptic vesicular release kinetics. By combining electrophysiological and ultrastructural analysis, we further assessed vesicular recycling properties on the synapse. This combination together with the use of mitochondrial inhibitors, such as oligomycin, allowed us to study the mechanism of RNS60 action on ATP synthesis (Lardy et al., 1958).

Synaptic noise was recorded using a Neurodata Instrument amplifier (ER-91) with a Butterworth filter (0.1-1 kHz). Noise analysis was based on postsynaptic spontaneous unitary waveform determination via two exponential functions (Verveen and DeFelice, 1974), F(t)=a[/[e−t/τd_e−t/τr] where a is an amplitude scaling factor and τd and τr are the decay and rise time constants respectively.

The power spectrum derived from the unitary potentials is S(f)=2na²(τd−τr)²/[1+4π²f²τ² d)(1+4π²f2τ²r)] where n is the rate of unitary release f and a, τd and τr are the same as above. The change in spontaneous release was quantified by averaging noise amplitude in noise frequencies between 20 and 200 Hz.

Noise Model.

In order to address the noise fluctuation changes observed following RNS60 based ASW we implemented a numerical solution for the noise profile (Lin et al., 1990). As in previous studies (Lin et al 1990), the time constant for the miniature potential rise time was determined as having a 0.2 ms and the fall time as 1.5 ms. The noise results following RNS60 were found to have a rise time of 0.2 and a fall time of 2.5 msec. The parameters for the RNS60 noise profile were selected by goodness of fit.

Voltage Clamp.

The voltage clamp experiments followed a standard protocol (Llinas et al. 1981). Briefly, two glass micropipette electrodes were inserted into the largest (most distal) presynaptic terminal digit at the synaptic junction site and a third micropipette impaled the postsynaptic axon at the junction site (Llinas R. et al 1981). One of the presynaptic electrodes was used for microinjection supporting the voltage clamp current feedback, while the second monitored membrane potential. Presynaptic voltage was measured using an FET input operational amplifier (Analog Devices model 515, Analog Devices, Inc., Norwood, Mass.). Current was injected by means of a high-speed, high-voltage amplifier (Burr-Brown Corp, 3584JM). Total current was measured by means of a virtual ground circuit (Teledyne Philbrick 1439, Teledyne Philbrick, Dedham, Mass.). The indifferent electrode consisted of a large silver-silver chloride plate located across the bottom of the chamber. To eliminate polarization artifacts, current was measured using an Ag—AgCl agar virtual ground electrode placed in the bath adjacent to the synapse. In most cases the time to plateau of the voltage microelectrode signal ranged from 50 to 150 μs.

ATP Synthesis.

ATP synthesis was determined using luciferin/luciferase light emitting measurements (McElroy W. D. 1947). Luciferase was pressure-injected into either the presynaptic or the postsynaptic terminal. Luciferin was added to the superfusate. Light emission was monitored and imaged using a single photon counting video camera (Argos −100 Hamamatsu Photonix). Light magnitude was determined using fifteen-second time integration periods. Oligomycin (0.25 mg/ml) was injected presynaptically using 50-100 ms pressure pulses and visualized directly using the photon counting camera. The volume injected was in the range of 0.5 to 1 pl, i.e., about 5 to 10% of the presynaptic terminal volume (Ulnas R. et al. 1991) for a final concentration of 25.0 μg/ml, to block ATP synthesis.

Block of ATP Synthesis with Oligomycin.

Oligomycin (0.25 mg/ml) was injected presynaptically using 50-100 ms pressure pulses and visualized directly using the photon counting camera. The volume injected was in the range of 0.5 to 1 pl, i.e. about 5 to 10% of the presynaptic terminal. volume (Llinas et al., 1991) for a final concentration of 25.0 μg/ml, to block ATP synthesis.

Ultrastructural Studies.

At the end of the electrophysiological recordings the stellate ganglion was immediately removed from the recording chamber and fixed by immersion in glutaraldehyde. Only synapses showing perfect preservation were accepted for analysis. Ultrastructural analysis was thus carried out on 240 active zones (AZ) from 8 synaptic terminals, as summarized in Table 1. The tissue was postfixed in osmium tetroxide, stained in block with uranium acetate, dehydrated and embedded in resin (Embed 812, EM Sciences). Ultrathin sections were collected on Pioloform (Ted Pella, Redding, Calif.) and carbon-coated single sloth grids, and contrasted with uranyl acetate and lead citrate. Morphometry and quantitative analysis of the synaptic vesicles were performed with the Image J software (NIH, EUA). Electron micrographs were taken at an initial magnification of 20 or 30K. They were enlarged on a computer screen to a magnification of 50K for counting synaptic vesicles and to 75K for counting clathrin-coated vesicles (CCV). Synaptic vesicle density and the number of CCV at the synaptic active zones were determined as the number of vesicles per μm2.

Statistics; Morphology.

The synaptic vesicle density was analyzed by one-way ANOVA test (parametric test) followed by the Tukey test, and the CCV density was analyzed by the Mann-Whitney U test (non-parametric test). Both analyzes were realized in the Statistical Analysis System Software 10.0 (Statistical Analysis System Institute Inc., EUA). The data is presented as average±standard error).

Electrophysiology.

Analysis of the electrophysiological data was carried out in the SPSS environment (SPSS Statistics, IBM). Several measurements of each parameter were made for each experiment. Statistical analysis was carried out on the grand mean of the mean for each synapse. The t-test or independent samples ANOVA followed by the Tukey post-hoc test were used to determine significance. Three statistical thresholds are marked, P<0.05, P<0.01, P<0.001.

Database.

The data for this study were obtained from a total of 75 squid synapses yielding eighty-five experiments as summarized in Table 1, Synapses were included for analysis only if they had stable presynaptic and postsynaptic resting potentials and if the presynaptic and postsynaptic action potentials did not show signs of deterioration under control conditions.

TABLE 1
Summary of experiments comprising database for this study.

| | | | Control | *Oligomycin | |
|---|---|---|---|---|---|
| | | Control | PNS50 | Control | |
| Type of Experiment | Control | RNS60 | RNS60 | RNS60 | Total |
| Low oxygen content | — | 10 | — | — | 10 |
| Evoked release: Single | — | 5 | — | — | 5 |
| stimulus | | | | | |
| Evoked release: | 4 | 9 | 5 | 7 | 25 |
| Recuperation from | | | | | |
| repetitive stimulation | | | | | |
| Spontaneous Release | 5 | 6 | 5 | 9 | 25 |
| (noise and analysis) | | | | | |
| Presynaptic voltage | ref | 6 | — | — | 6 |
| clamp | | | | | |
| Intracellular ATP | — | 10 | — | — | 10 |
| generation | | | | | |
| (luciferin/luciferase) | | | | | |
| Total | 9 | 46 | 10 | 16 | 81 |

Oligomycin was injected into the synapse.

### Example 9

### Electrophysiological Studies were Performed, and Showed that RNS60 ASW Rescued Synaptic Transmission from Low Oxygen Block

Initial experiments tested the ability of presynaptic activation to generate a post synaptic response (Hagiwara S. and Tasaki, I, 1958 Takeuchi A. and Takeuchi N. 1962, and Kusano K. 1968) in the presence of physically modified ASW (RNS60 ASW) versus control ASW. In all of the synapses studied, superfusion with RNS60 ASW enhanced synaptic transmission. RNS60 ASW did not modify the resting membrane potential of the presynaptic membrane (Table 3). This was the case after intracellular injection of luciferase into the presynaptic terminal. RNS60 ASW did hyperpolarize the postsynaptic resting potential. This was most likely due to increased activity of the Na—K ATPase due to increased APT availability in the presence of RNS60. Membrane hyperpolarization was not seen when luciferase was injected into the postsynaptic terminal (Table 3).

RNS60 ASW Rescued Synaptic Transmission from Low Oxygen Block.

As originally demonstrated by Bryant S H. (1958) and Colton C A. et al (1992), when synapses are not properly oxygenated synaptic transmission fails within 30 min. This is due to transmitter depletion following hypoxia (Colton C A. et al., 1992). An initial set of experiments was, therefore, designed to determine if RNS60 could restore normal transmission in hypoxic synapses.

Initial experiments, providing a simple direct test of RNS60 ability to restore synaptic transmission relative to a Control low oxygen ASW, consisted of allowing postsynaptic amplitude to decline such that only small, subthreshold postsynaptic synaptic potentials could be elicited (FIG. 10, lower arrow). When the hypoxic synapse was superfused with RNS60 (RNS60 ASW) the postsynaptic potential rapidly increases in amplitude to the point that a postsynaptic spike could be easily evoked by each presynaptic stimulus. The action potential in FIG. 10 was recorded three minutes after changing to RNS60 (RNS60 ASW). Such recordings could be made with long-term superfusion of RNS60 (RNS60 ASW), up to several hours. This demonstrates that RNS60 (RNS60 ASW) can rapidly and effectively restore transmission after hypoxic failure and does not itself have a deleterious effect on the transmission event as seen with oxygenated ASW (Colton et al., 1992).

FIG. 10 shows, according to particular exemplary aspects, an example of increased evoked transmitter release in a hypoxic synapse following electrical stimulation of the presynaptic terminal. Note the small subthreshold synaptic potential after 30 minutes of hypoxia and the action potential elicited 3 minutes after superfusion with RNS60 ASW. Insert is an amplitude magnification (×3) showing detail of the EPSP onset indicating change in amplitude without a change in release latency. Time, amplitude and postsynaptic fiber resting potential are as indicated.

### Example 10

### RNS60 ASW Rescued Transmission from High Frequency Stimulation Synaptic Fatigue

Following the demonstration that no long-term changes occurred with superperfusion with RNS60 ASW, a study of transmitter depletion following repetitive stimulation was carried out. High frequency stimulation of the squid giant synapse leads to a reduction of synaptic vesicles and failure of postsynaptic spike generation that can be restored after a period of rest (Kusano K. and Landau 1975, Weight F F. and Erulkar S. D., 1976; Gillespie I. J., 1979).

A set of experiments was designed to determine if RNS60 altered the time course of recovery from such synaptic fatigue. Trains of 50 tetanic stimuli (at 200 Hz) were applied every second until synaptic failure (no postsynaptic spike) occurred. The synapse was then allowed to rest and the stimulus train was again applied. The number of spikes elicited during each train were used as an indication of synaptic failure or recovery, the latter providing a quantitative measure of intracellular transmitter replenishment. This protocol was followed in Control ASW and in RNS60 ASW as shown in the example illustrated in FIG. 11.

FIGS. 11A-11E show, according to particular exemplary aspects, high-frequency stimulation in Control and RNS60 ASW. FIG. 11A shows presynaptic (red) and postsynaptic (black) spikes generated by a repetitive presynaptic electrical stimulation at 200 Hz (note the last stimulus fails to generate a post synaptic spike). FIG. 11B shows failure of all postsynaptic spike generation after 100 consecutive trains repeated at 1 Hz in Control ASW. FIG. 11C shows same as in B, but recorded in RNS60 ASW. FIG. 11D shows partial recovery of postsynaptic spike generation after a 30 second rest period in Control ASW. FIG. 11E shows partial recovery after rest period in RNS60 ASW. Note in D and E that in the presence of RNS60 ASW there was a more vigorous recovery of postsynaptic spike generation after a similar 30 sec rest period than in Control ASW. Similar results were obtained in four other synapses utilizing the same stimulus paradigm.

In Control ASW, the squid giant synapse can follow transmission at a stimulation rate of 200 Hz. As shown in FIG. 11A, a 200 Hz stimulation train elicited a presynaptic action potential (black) and a postsynaptic action potential (red) for the first 49 of 50 stimuli. However, when such trains were delivered at 1 Hz, transmission failed in Control ASW (FIG. 11B) and in RNS60 ASW (FIG. 11C). A difference was seen in the time course of recovery in the Control and RNS60 ASW. In example in FIG. 11 in the Control ASW, after a 30 sec rest period the first 12 stimuli of the train elicited a postsynaptic spike after which only subthreshold EPSPs were elicited (FIG. 211). However, following RNS60 ASW, the first 22 stimuli elicited a postsynaptic spike (FIG. 11E).

As this simple test allowed a first approximation methodology to test recovery from hypoxia, two types of experiments were implemented: 1) Recovery from repetitive stimulation in non-artificially oxygenated (control) ASW, or 2) recovery in the presence of RNS60 ASW. The mean recovery in control ASW was 14±2.5% (n=4) and that in RNS60 ASW was 68±6.2% (n=9). Statistical analysis revealed that the type of ASW had a significant effect on recovery (T(1,12)=6.26, p<0.0001).

These findings indicate that there was also an increase in transmitter availability in addition to an increase in the amount of transmitter (as indicated by the increased EPSP amplitude), during RNS60 ASW superfusion. This suggests that vesicular recycling may be modified, allowing rapid vesicular turnover and increased transmitter availability.

### Example 11

### RNS60 ASW Increased Spontaneous Transmitter Release

A related set of measurements of transmitter availability and release kinetics may be obtained by determining the magnitude of spontaneous transmitter release (Miledi R., 1966, Kusano K. and Landau E. M., 1975, Mann D. W. and Joyner R. W., 1978, Lin J. W. et al, 1990) in the squid synapse. This measurement has often been utilized as a measure of vesicular availability at a given junction (Lin J. W. et al., 1990).

To determine whether RNS60 can modify such spontaneous release, synaptic noise was measure in Control ASW and after superfusion with RNS60 ASW (FIG. 12). FIG. 12A shows that synaptic noise recorded 5 minutes (FIG. 12A, red trace) and 10 minutes (FIG. 12A, blue trace) after superfusion with RNS60 ASW was greater than that recorded in Control ASW (FIG. 12 A, green trace). Fast Fourier Transform (FFT) analysis of the synaptic noise showed that the increased spontaneous release occurred at frequencies over 200 Hz (FIG. 12B). This consistent with the function predicted by a model (FIG. 12B, insert).

FIGS. 12A-12C show, according to particular exemplary aspects, synaptic noise recorded in Control ASW and RNS60 ASW. FIG. 12A shows recordings showing synaptic noise across the postsynaptic membrane superfused with Control ASW (green) and the increase in noise amplitude 5 min (red) and 10 min (blue) after superfusion with RNS60 ASW as well as the background extracellular noise recorded directly from the bath (black). FIG. 12B shows a plot of change in noise amplitude as a function of time for after superfusion with RNS60 ASW. FIG. 12C shows a plot of noise amplitude as a function of frequency (note log scale) in Control ASW (red) and 10 min after superfusion with RNS60 ASW (black). The insert shows model results indicating that the change in noise plotting could be interpreted as a change in the time course and amplitude of synaptic miniature noise. (e.g., for details see Lin et al., 1990.)

These results indicate a significant increase of spontaneous transmitter release, ranging from 20% to 80% that optimized about ten minutes after changing from Control to RNS60 ASW. This is shown for four synapses in FIG. 12C where synaptic noise is plotted as a function of time after changing to RNS60 ASW. This increase level of spontaneous transmitter release was maintained for the duration of the experiments, up to 25 minutes, in accordance with the findings shown in FIGS. 12 and 11.

### Example 12

### Presynaptic Calcium Current Modulation was Shown not to Mediate Increased Transmitter Release

The results discussed above indicate that superfusion with RNS60 ASW results in an increase in both evoked and spontaneous transmitter release that is possibly related to transmitter availability. Importantly, it also suggests that this increase does not elevate transmitter release beyond an optimal functional level.

While such findings may be the result of any of the many components of the release process, one possible candidate is changes in presynaptic ionic channel kinetics following RNS60 ASW superfusion. Of these, the most likely would be modulation of presynaptic voltage-gated calcium current (ICa⁺⁺). An increase in this parameter could explain many of the results described so far. Indeed, an increase in ICa⁺⁺ would influence the degree of transmitter release by increasing the probability of vesicular fusion at the presynaptic terminals well as an increase spontaneous transmitter release. Given the possibility of implementing a presynaptic voltage clamp paradigm, (Llinas R. et al., 1976, 1981, Augustine G J. et al., 1986) this synapse is optimal as a research tool to address changes in presynaptic calcium currents.

A set of voltage clamp experiments was implemented to determine if the RNS60 modulation of transmitter release seen above is mediated by an increase in the presynaptic calcium current. A second issue to consider was whether the relation between ICa⁺⁺ and transmitter release (Llinas et al., 1981) was maintained or otherwise modified by the presence on RNS60.

Presynaptic calcium currents were elicited by graded depolarizing step pulses after pharmacological block of the voltage-gated sodium and potassium conductances (Llinas et al., 1976; 1981a; Augustine and Charlton, 1986). FIG. 13A illustrates the presynaptic calcium current (Pre ICa), postsynaptic EPSP, and presynaptic voltage pulse (PreV) at three levels of presynaptic depolarization in control (top traces, green) and RNS60 (bottom traces, red) ASW. The calcium current and EPSP traces are superimposed in FIG. 13B. It is immediately apparent that the postsynaptic response amplitude was larger in RNS60 (red) than in Control (green) ASW and that presynaptic inward calcium current was not significantly modified by RNS60. Note that the difference between the control and RNS60 EPSPs for the largest presynaptic depolarization is less than that for the middle depolarization. This is because the presynaptic membrane is close to the equilibrium potential for calcium, reducing ICa++ and the EPSP amplitude (Llinas et al., 1981a). The EPSP amplitude is plotted in FIG. 13C for five synapses as a function of presynaptic voltage clamp depolarization. Each synapse has a different marker and the EPSPs recorded in Control ASW (green) RNS60 ASW (red) may be compared for each synapse. Note that the increase in transmitter release varied among synapses, but in every case was larger in the RNS60 ASW and reached a maximum value. Once this value was attained, we did not observe any further increase with protracted superfusion, suggesting that conditions for enhanced transmitter release had been reached. When the mean amplitude of the postsynaptic response in control and RNS60 ASW were compared, significant differences were seen at three levels of depolarization. As may be seen in FIG. 13D, depolarizing pulses were not exactly the same amplitude across synapses. To calculate the mean EPSP amplitude, the responses were assigned to one of four groups according to the presynaptic depolarization (two depolarization values, 16.5 mV and 25 mV, were not included a group). There was a significant difference in EPSP recorded in control and RSN60 ASW in three presynaptic depolarization groups: 38 mV, (T(1,8)=4.27, p<0.01); 43 mV, (T(1,8)=5.1, p<0.001), 48 mV, (T(1,8)=3.54, p<0.01). RNS60 did not change the decay constant of the EPSPs. This suggests that there was not a significant change in the passive properties (resistance or capacitance) of the postsynaptic membrane (τ, control 2.99±0.7 msec; RNS60 2.36±0.3 msec, n=9).

Thus, the results from five voltage clamp experiments clearly indicate that the increase in transmitter release was not accompanied by a modification of calcium current kinetics or magnitude. At this point the possibility was considered that the effect of RNS60 could be related to some aspect of vesicular availability and related intracellular vesicular dynamics.

Of significance here is also the fact that when compared with similar voltage clamp results in past experiments (Llinas et al., 1981) (FIGS. 13D and E, black) performed with oxygenated sea water, those results superimposed on our present control. This indicates that the increase in transmitter release following RNS60 based ASW increases transmitter release beyond that expected from normally oxygenated sea water.

FIGS. 13A-13E show, according to particular exemplary aspects, a voltage clamp study indicating that RNS60 increases transmitter release without modifying calcium current or its relationship with transmitter release. FIG. 13A shows a set of traces recorded in Control ASW showing the amplitude and time course of the presynaptic calcium current (black), the amplitude and time course of the postsynaptic response (green) elicited by the rapid voltage clamp step shown in the third trace (Pre Dep, black). FIG. 13B shows a set of traces recorded in RNS60 ASW with the same amplitude depolarizing pulses as in the control set; EPSPs are red. FIG. 13C shows superposition of calcium currents (upper traces) and EPSPs (lower trace) from panel A for control (green) and panel B for RNS60 (red) ASW, demonstrating that there was no change in the time course or amplitude of the presynaptic calcium current, but a clear increase in the EPSP amplitude in RNS60 compared to Control ASW. FIG. 13D shows a plot of EPSP amplitude as a function of presynaptic depolarization step for the five synapses (the set of recordings from each synapse use the same marker). FIG. 13E shows a plot of mean EPSP mean and s.e.m. for synapses in panel D (*P<0.05, **P<0.005, t-test).

### Example 13

### An RNS60-Mediated Increase of ATP Synthesis at the Presynaptic and Postsynaptic Terminals was Determined Using Luciferin/Luciferase Light Emission

A set of experiments was designed to determine the time course and magnitude of any change in ATP levels when the superfusate was changed from Control to RNS60 ASW. ATP levels were measured using the luciferin/luciferase protocol in which there is a direct correlation between light emission and ATP levels (Spielmann, H et al., 1981). Light measurements were made in both the presynaptic and postsynaptic elements of the synapse.

FIGS. 14A-14F show, according to particular exemplary aspects, direct determination of increased ATP synthesis at the presynaptic and postsynaptic terminals using Luciferin/Luciferase light emission. FIG. 14A shows the levels of luciferin/luciferase light emission at control (Cont.) and at 3 and 6 minutes following RNS60 superfusion. Note in FIGS. 14B, 14C, and 14D that the amplitude and resting potential recorded at the postsynaptic axon increased indicating an optimization of postsynaptic axon viability that is in phase with the increased level of ATP measured at the presynaptic terminal following RNS60 ASW. A similar increase in ATP level could also be observed at the postsynaptic axon under similar conditions as illustrated in FIGS. 14E and 14F. In FIG. 14E, pre (green) and postsynaptic (red) elements are drawn. The luciferase injected site at the postsynaptic terminal is marked in white. In FIG. 14F the light emission is shown after two and five minutes following RNS60 superfusion.

More specifically, there was a clear increase in ATP levels from control levels (FIG. 14A, Cont) as indicated by the increased light emission recorded three and six minutes after the superfusate was changed from Control to RNS60 ASW (FIG. 14A). During this same period there was a small decrease in the resting potential of the presynaptic terminal, but no change in the action potential amplitude (FIG. 14B-D). There was a small increase in the resting potential in the postsynaptic axon between 3 and 6 minutes after staring RNS60 superfusion. Unlike the presynaptic element, there was increase in the amplitude of the postsynaptic action potential (FIG. 14B-D). The results indicate that the increase in synaptic transmission following RNS60 superperfusion is accompanied by an increase in ATP levels in both the presynaptic and postsynaptic terminals.

### Example 14

### Oligomycin, an ATP Synthesis Blocker, Blocked RNS60-Mediated Increase of ATP Synthesis at the Presynaptic and Postsynaptic Terminals

One clear possibility to be addressed is whether the properties of RNS60 facilitated access of oxygen to intracellular compartments more efficiently than dissolved oxygen. If this were the case, one immediate possibility was that RNS60 ASW could support ATP synthesis more efficiently than diffusion-oxygenated ASW and thus increase vesicular availability either by increasing clathrin activity (Augustine G J. et al 2006) or by non-clathrin dependent vesicular endocytosis (Daly C. et al 1992). Given this possibility, a set of experiments was design to test whether blocking ATP synthesis by interfering with mitochondrial function induced by hypoxia (Jonas E A, 2004; Jonas E A, et al., 2005)) would prevent modified synaptic transmitter release by RNS60 as seen in FIGS. 1-5.

A reduction of ATP would be expected to reduce transmitter release since many aspects of synaptic vesicle mobilization and recycling are mitochondrial ATP dependent (reviewed in Vos et al., 2010). Although several of the effects of mitochondrial blockade on synaptic transmission are extracellular calcium concentration related (Talbot 2003).

Mitochondria can be blocked with drugs that do not alter mitochondrial membrane potential (Ψₘ), or with depolarizing Ψₘ inhibitors. Mitochondrial depolarizing agents affect both ATP production and mitochondrial calcium uptake. It is proposed that most of the effects observed in synaptic transmission by depolarizing Ψₘ inhibitors are related to changes in calcium dynamics at the presynaptic terminal (Billups and Forsythe et al., 2010, Talbot et al., 2003). Oligomycin was selected for use in the present studies, because it inhibits ATP synthase but does not depolarize mitochondria, and is reported to have no effect on either cytosolic or mitochondrial calcium dynamics in several preparations but acts by blocking complex V (David 1999, Talbot et al., 2003).

The most sensitive measure of vesicular turnover and the overall release apparatus is spontaneous transmitter release as it involves the least number of steps in its activation. With this in mind, a set of experiments was implemented to determine the effect of blocking ATP syntheses on spontaneous transmitter release.

FIG. 15 shows, according to particular exemplary aspects, reduction of spontaneous synaptic release following oligomycin administration; plots of noise amplitude as a function of frequency (note double log coordinates). Red is Control ASW, green is 7 min after addition of oligomycin and blue is 22 min after oligomycin administration and 12 min after changing superfusion to RNS60 ASW. Black is extracellular recording.

Specifically, presynaptic intracellular oligomycin injection (0.25 mg/ml) during Control ASW superfusion markedly reduced spontaneous release from control levels (compare FIG. 15, red and green). This occurred rapidly in all experiments. A reduction of more than an order of magnitude occurred within the first seven minutes after oligomycin injection into the presynaptic terminal. Changing the superfusion to RNS60 ASW 22 min after injection of oligomycin failed to increase spontaneous transmitter release (FIG. 15, blue). The blue curve in FIG. 15 was recorded 12 minutes after the start of RNS60 ASW superfusion. Similar findings in were seen in 5 experiments. Thus, RNS60 ASW failed to rescue synaptic transmission from the reduction due to ATP depletion.

FIGS. 19A-19C show, according to particular exemplary aspects, the effect of RNS60 and olygomycin on synaptic vesicle numbers. FIG. 19A shows the number of lucid small synaptic vesicles after superfusion with control (green), RNS60 (red) and RNS60 and presynaptic injection of oligomycin (blue). FIG. 19B shows the number of large, irregular vesicles under the same three conditions as in panel A. FIG. 19C the number of clatherin-coated vesicles under the same three conditions as in panel A. *<0.05, Mann-Witney.

There was a statistically significant decrease in SSV number in RNS60 ASW superfused terminals compared (FIG. 19A, red) with control terminals (FIG. 19A, green) F (1.114)=5.97, p<0.05). By contrast, the number of CCVs was higher in RNS60 (FIG. 19C red) than control (FIG. 19C, green) synapses but this difference did not reach significance. In addition, the increased number of large vesicles suggests an increased vesicular turnover, as would be expected from an increased ATP level at the presynaptic terminal. These results are in accordance with research on the relation between mitochondria and vesicular formation and availability (Ivanikov et al., 2010).

### Example 15

### Three Primary Differences with Normal Morphology were Noticed at the Synaptic Active Zone Following RNS60 Administration: 1) an Increase in the Number of Clathrin-Coated Vesicles (CCV), 2) Increase in the Number of Large Diameter Vesicles, and 3) a Reduction of the Numbers of Regular-Sized Synaptic Vesicles at the Active Zone, Suggesting Increased Release Dynamics

Ultrastructural Analysis of RNS60 Treated Synapses.

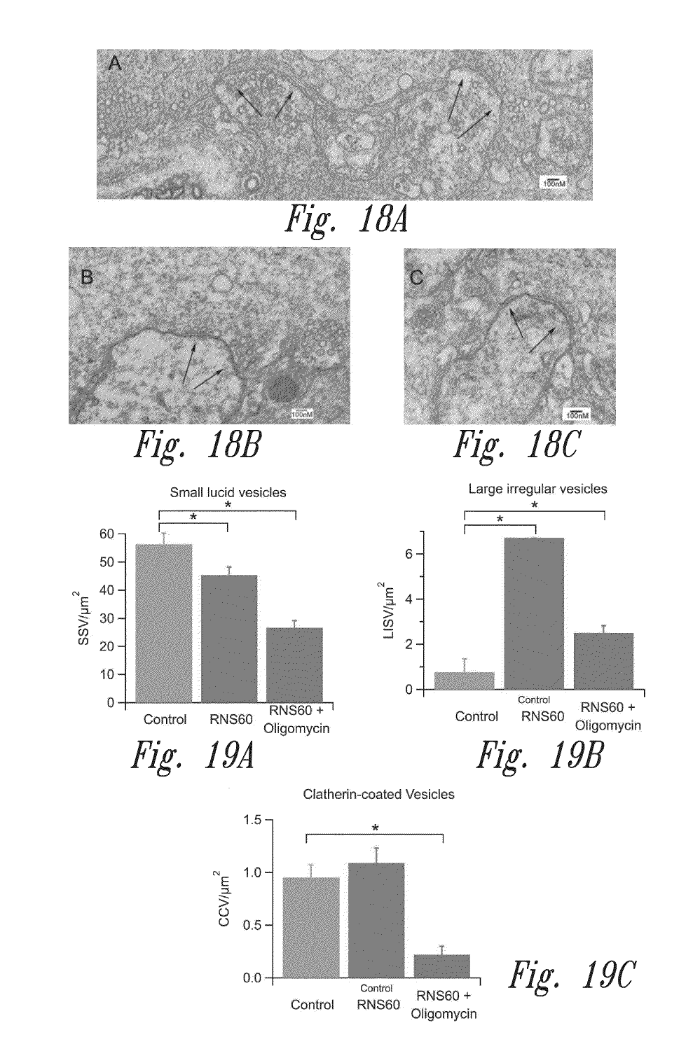

Electron microscopic analysis of presynaptic and postsynaptic morphology revealed very well preserved ultrastructural changes following RNS60 ASW administration. In general terms, the ultrastructure demonstrated well preserved cytosolic properties as well as mitochondrial profiles (FIGS. 16 and 18). The number of synaptic vesicles and CCV were analyzed in 1 μm² of each active zone. Quantification was carried out in 20-25 active zones in 2 control synapses and 3 RNS60 ASW synapses.

Concerning synaptic morphology three main differences with normal morphology were noticed at the synaptic active zone following RNS60 administration: 1) an increase in the number of clathrin-coated vesicles (CCV), 2) increase in the number of large diameter vesicles (LEV), and 3) a reduction of the numbers of lucid, regular-sized synaptic vesicles (SSV) at the active zone, suggesting increased release dynamics.

There was a statistically significant decrease in SSV number in RNS60 ASW superfused terminals compared with control terminals (FIG. 16A, red and green). By contrast, the number of CCVs was higher in RNS60 than control (FIG. 16B, red and green) synapses but this difference did not reach significance. In addition, a large increase in the number of large vesicles (FIG. 16C, red and green) suggests an increased vesicular turnover, as would be expected from an increased ATP level at the presynaptic terminal. These results are in accordance with our research on the relation between mitochondria and vesicular formation and availability (Ivannikov et al., 2010).

Specifically, FIGS. 16A-16C show, according to particular exemplary aspects, electronmicrographs of a synaptic junction following RNS60 ASW superfusion. FIG. 16A shows vesicles of irregular shapes and sizes are present in the terminals. Blue dots denote large synaptic like vesicles, and red dots denote mark clathrin-coated vesicles. FIG. 16B shows a lower-magnification presynaptic and postsynaptic image, showing postsynaptic digit making several contacts forming active zones with the presynaptic terminal (yellow dots). FIG. 16C shows a large increase in the number of large vesicles (FIG. 16C, red and green).

FIGS. 8A and 8B show, according to particular exemplary aspects, statistical determination of synaptic vesicle numbers in synapses superfused with RNS60 ASW. FIG. 17A shows a plot of the number of CCV as a function of size. FIG. 17B shows the number of large vesicles as a function of size.

Of interest is the fact that a direct comparison of the release site ultrastructure in a study from over 70 different active zones in the presence and absence of RSN60 has revealed that in the presence of RNS60 ASW the number of normal synaptic vesicles is significantly decreased. In addition, the number of large vesicles suggests an increased vesicular turnover, as would be expected from an increased ATP level at the presynaptic terminal. These results are in accordance with research on the relation between mitochondria and vesicular formation and availability (Ivanikov et al., 2010).

Block of ATP Synthesis with Oligomycin Prevents Effects of RNS60.

In synapses treated with oligomycin the results from ultrastructural analysis indicate a marked reduction in all synaptic vesicle types. Indeed, images from such synapses (FIG. 18) indicate that while the ultrastructure is not grossly altered the numbers of vesicles of all types in the vicinity of the active zones are very much reduced.

FIGS. 18A-18C show, according to particular exemplary aspects, the ultrastructure of squid giant synapse active zones following oligomycin injection. In FIGS. 18A-18C, black arrows indicate active zones showing few, if any, synaptic vesicles. Note also the lack of CCV and of large vesicular profiles that are generally found in the presence of synapses superfused with RNS60 ASW. Note also the presence of few vesicles scattered away from the active zone (red arrow).

The actual numbers of vesicles were quantified from four synapses and a total of different 180 active zones examined.

In Summary of Enhanced Synaptic Transmission Aspects.

Determining the biological variables that control both electrical and chemical synaptic transmission between nerve cells, or between nerve terminals and muscular or glandular systems, has been a very significant area of physiological exploration over the decades. Chemical synaptic transmission has had the added attraction of addressing both the transmission gain of the event, as well as the excitatory or inhibitory nature of the junction and its activity-dependent potentiation or depression.

According to particular aspects, exposure of neurons to an electrokinetically-altered ionic aqueous solution comprising charge-stabilized oxygen-containing nanostructures (e.g., oxygen nanobubbles) (e.g., RNS60; a physically modified isotonic saline prepared in accordance with Applicants' U.S. Pat. Nos. 7,832,920, 7,919,534, 8,410,182, 8,445,546, 8,449,172, and 8,470,893) generates an optimization of synaptic transmission in neurons, for example, as exemplified by synaptic transmission at the squid giant synapse (superfused with artificial seawater (ASW) based on isotonic saline comprising oxygen nanobubbles (RNS60 ASW). This was determined by examining the postsynaptic response to single and repetitive presynaptic spike activation, spontaneous transmitter release, and presynaptic voltage clamp studies. This optimization of synaptic transmission reached stable maxima within 5 to 10 minutes following superfusion with the RNS60-based ASW.

Analysis of synaptic noise at the post-synaptic axon during RNS60 ASW superfusion revealed an increase of spontaneous transmitter release with a modification of noise kinetics. This increase was maintained for the duration of the recording time, usually one hour. Synaptic release was assessed by electrical activation of presynaptic action potentials, either as single events or following 200 Hz repetitive presynaptic stimulation. Voltage clamp of the presynaptic terminal demonstrated an increase in postsynaptic response, without an increase in presynaptic ICa⁺⁺ amplitude during RNS60 ASW superfusion. Electronmicroscopic based morphometry indicated a decrease in synaptic vesicle density and number at active zones with an increase in the number of clathrin-coated vesicles, and large endosome like vesicles in the vicinity of the junctional sites. Finally, block of mitochondrial ATP synthesis by presynaptic injection of oligomycin markedly reduced spontaneous release and prevented the synaptic noise increase seen in RNS60 ASW. At the ultrastructural level there was a large reduction of vesicles at the active zone at the presynaptic junction as well as a reduction in the number of clathrin-coated vesicles with an increase in large vesicles. The possibility that RNS60 ASW acts by increasing mitochondrial ATP synthesis was tested by direct determination of ATP levels in both presynaptic and postsynaptic structures. This was implemented using luciferin/luciferase photon emission, which demonstrated a marked increase in ATP synthesis following RNS60 administration. Without being bound by mechanism, RNS60 likely positively modulates synaptic transmission by up-regulating ATP synthesis leading to synaptic transmission optimization.

References; incorporated herein by reference in their respective entireties:
Whitehouse, P. J., Price, D. L., Struble, R. G., Clark, A. W., Coyle, J. T., and Delon, M. R. (1982) Science 215, 1237-1239
2. Colangelo, V., Schurr, J., Ball, M. J., Pelaez, R. P., Bazan, N. G., and Lukiw, W. J. (2002) J Neurosci Res 70, 462-473
3. Jacob, C. P., Koutsilieri, E., Bartl, J., Neuen-Jacob, E., Arzberger, T., Zander, N., Ravid, R., Roggendorf, W., Riederer, P., and Grunblatt, E. (2007) J Alzheimers Dis 11, 97-116
4. Myers, S. J., Dingledine, R., and Borges, K. (1999) Annu Rev Pharmacol Toxicol 39, 221-241
5. Selkoe, D. J. (2002) Science 298, 789-791
6. Shim, K. S., and Lubec, G. (2002) Neurosci Lett 324, 209-212
7. Clayton, D. A., Mesches, M. H., Alvarez, E., Bickford, P. C., and Browning, M. D. (2002) J Neurosci 22, 3628-3637
8. Malenka, R. C., and Bear, M. F. (2004) Neuron 44, 5-21
9. Collingridge, G. L., Peineau, S., Howland, J. G., and Wang, Y. T. Nat Rev Neurosci 11, 459-473
10. Olney, J. W., Wozniak, D. F., and Farber, N. B. (1998) Restor Neurol Neurosci 13, 75-83
11. Mishizen-Eberz, A. J., Rissman, R. A., Carter, T. L., Ikonomovic, M. D., Wolfe, B. B., and Armstrong, D. M. (2004) Neurobiol Dis 15, 80-92
12. Desjardins, S., Mayo, W., Vallee, M., Hancock, D., Le Moal, M., Simon, H., and Abrous, D. N. (1997) Neurobiol Aging 18, 37-44
13. Proctor, D. T., Coulson, E. J., and Dodd, P. R. J Alzheimers Dis 21, 795-811
14. Reddy, P. H., Mani, G., Park, B. S., Jacques, J., Murdoch, G., Whetsell, W., Jr., Kaye, J., and Manczak, M. (2005) J Alzheimers Dis 7, 103-117; discussion 173-180
15. Bell, K. F., Zheng, L., Fahrenholz, F., and Cuello, A. C. (2008) Neurobiol Aging 29, 554-565
16. Good, P. F., Werner, P., Hsu, A., Olanow, C. W., and Perl, D. P. (1996) Am J Pathol 149, 21-28
17. Lipton, S. A., Choi, Y. B., Pan, Z. H., Lei, S. Z., Chen, H. S., Sucher, N. J., Loscalzo, J., Singel, D. J., and Stamler, J. S. (1993) Nature 364, 626-632
18. Selvakumar, B., Jenkins, M. A., Hussain, N. K., Huganir, R. L., Traynelis, S. F., and Snyder, S. H. Proc Natl Acad Sci USA 110, 1077-1082
19. Khasnavis, S., Jana, A., Roy, A., Mazumder, M., Bhushan, B., Wood, T., Ghosh, S., Watson, R., and Pahan, K. (2012) J Biol Chem 287, 29529-29542
20. Mondal, S., Martinson, J. A., Ghosh, S., Watson, R., and Pahan, K. (2012) PLoS One 7, e51869
21. Jana, M., Jana, A., Pal, U., and Pahan, K. (2007) Neurochemical research 32, 2015-2022
22. Saha, R. N., Ghosh, A., Palencia, C. A., Fung, Y. K., Dudek, S. M., and Pahan, K. (2009) J Immunol 183, 2068-2078
23. Roy, A., Jana, A., Yatish, K., Freidt, M. B., Fung, Y. K., Martinson, J. A., and Pahan, K. (2008) Free Radic Biol Med 45, 686-699
24. Mondal, S., Roy, A., Jana, A., Ghosh, S., Kordower, J. H., and Pahan, K. J Neuroimmune Pharmacol 7, 544-556
25. Ghosh, A., Roy, A., Liu, X., Kordower, J. H., Mufson, E. J., Hartley, D. M., Ghosh, S., Mosley, R. L., Gendelman, H. E., and Pahan, K. (2007) Proc Natl Acad Sci USA 104, 18754-18759
26. Roy, A., Ghosh, A., Jana, A., Liu, X., Brahmachari, S., Gendelman, H. E., and Pahan, K. PLoS One 7, e38113
27. Roy, A., Fung, Y. K., Liu, X., and Pahan, K. (2006) J Biol Chem 281, 14971-14980
28. Li, L., Stefan, M. I., and Le Novere, N. PLoS One 7, e43810
29. Impey, S., and Goodman, R. H. (2001) Sci STKE 2001, pe1
30. Bito, H., and Takemoto-Kimura, S. (2003) Cell Calcium 34, 425-430
31. Xia, Z., and Storm, D. R. Learn Mem 19, 369-374
32. Soderling, T. R. (1999) Trends Biochem Sci 24, 232-236
33. Franke, T. F., Kaplan, D. R., Cantley, L. C., and Toker, A. (1997) Science 275, 665-668
34. Koyasu, S. (2003) Nat Immunol 4, 313-319
35. Isaac, J. (2001) Neuron 32, 963-966
36. Danysz, W., and Parsons, C. G. (2003) Int J Geriatr Psychiatry 18, S23-32
37. Geerts, H., and Grossberg, G. T. (2006) J Clin Pharmacol 46, 8S-16S
38. Rossom, R., Adityanjee, and Dysken, M. (2004) Am J Geriatr Pharmacother 2, 303-312
39. Grinevich, V., Seeburg, P. H., Schwarz, M. K., and Jezova, D. Endocr Regul 46, 153-159
40. MacDonald, J. F., Jackson, M. F., and Beazely, M. A. (2006) Crit Rev Neurobiol 18, 71-84
41. Medina, A. E., Liao, D. S., Mower, A. F., and Ramoa, A. S. (2001) Neuron 32, 553-555
42. Yin, J. C., Del Vecchio, M., Zhou, H., and Tully, T. (1995) Cell 81, 107-115
43. Lee, H. K., Takamiya, K., Han, J. S., Man, H., Kim, C. H., Rumbaugh, G., Yu, S., Ding, L., He, C., Petralia, R. S., Wenthold, R. J., Gallagher, M., and Huganir, R. L. (2003) Cell 112, 631-643
44. Guzowski, J. F., and McGaugh, J. L. (1997) Proceedings of the National Academy of Sciences of the United States of America 94, 2693-2698
45. Impey, S., Smith, D. M., Obrietan, K., Donahue, R., Wade, C., and Storm, D. R. (1998) Nature neuroscience 1, 595-601
Attwell, D., Langlin, S. B., An energy budget for signaling in the grey matter of the brain., J Cereb Blood Flow Metab., 2001, 21(10), 1133-1145.
Augustine, G. J. and Charlton, M. P., Calcium dependence of presynaptic calcium current and post-synaptic response at the squid giant synapse. The Journal of physiology, 1986. 381: p. 619-640.
Augustine, G. J., Morgan, J. R., Villalba-Galea, C. A., Jin, S., Prasad, K., Lafer, E. M., Clathrin and synaptic vesicle endocytosis: studies at the squid giant synapse. Biochemical Society transactions, 2006. 34(Pt 1): p. 68-72.
Billups, B., Forsythe, I. D., Presynaptic mitochondorial calcium sequestration influences transmission at mammalian central synapses. J. Neurosci. 2002, 22(14), 840-847.
Burns, M. E., Sasaki, T., Takai, Y., Augustine, G. J., Rabphilin-3A: A multifunctional regulator of synaptic vesicle traffic, J. Gen. Physiol., 1998, 111(2), 243-255.
Colton, C., Yao, J. Grossman, Y., The effect of xanthine/xanthine oxidase generated reactive oxygen species on synaptic transmission. Free radical research communications, 1991. 14(5-6): p. 385-393.
Colton, C. A., J. S. Colton, and Gilbert, D. L., Oxygen dependency of synaptic transmission at the squid Loligo pealei giant synapse. Comparative biochemistry and physiology. Comparative physiology, 1992. 102(2): p. 279-283.
Daly, C., Sugimori, M., Moreira, J. E., Ziff, E. B., Llinas, R., Synaptophysin regulates clathrin-independent endocytosis of synaptic vesicles. PNAS, 2000. 97(11): p. 6120-6125.
David G., Mitochondrial clearance of cytosolic Ca²⁺ in stimulated lizard motor nerve terminals proceeds without progressive elevation of mitochondrial matrix [Ca²⁺] J Neurosci. 1999; 19:7495-7506.
Froesch, D. and Martin, R., Heterogeneity of synaptic vesicles in the squid giant fibre system. Brain research, 1972. 43(2): p. 573-579.
Fukuda M., Moreira, J. E., Lewis, F. M., Sugimori, M., Niinobe, M. Mikoshiba, K., Llinas, R., Role of the C2B domain in vesicular release and recycling as determined by specific antibody injection in the squid giant synapse pretierminal., PNAS, 1995, 92(23), 10708-10712.
Gillespie, J. I., The effect of repetitive stimulation on the passive electrical properties of the presynaptic terminal of the squid giant synapse. Proceedings of the Royal Society of London. Series B, Containing papers of a Biological character. Royal Society, 1979. 206(1164): p. 293-306.
Hagiwara, S. and Tasaki, I., A study on the mechanism of impulse transmission across the giant synapse of the squid. The Journal of physiology, 1958. 143(1): p. 114-37.
Hayashi M., Raimondi A. O'Toole E., Paradise S., Collesi C. Cremona O. Ferguson S. M. and De Camili P. Cell and stimulus-dependent heterogeneity of synaptic vesicle endocytic recycling mechanisms revealed by studies of dynamin 1-null neurons. PNAS 105: 2175-2180, 2008.
Heuser, J. E., Roose, T. S., Evidence for recycling of synaptic vesicle membrane during transmitter release at the frog neuromuscular junction., J. Cell Biol., 1973, 57(2), 317-331.
Ivannikov, M. V., Sugimori, M. and R. R. Llinas. Calcium Clearance and its energy requirement in cerebellar neurons, Cell Calcium 2010, 47: p. 507-513.
Jonas, E. A., Regulation of synaptic transmission by mitochondrial ion channels. Journal of bioenergetics and biomembranes, 2004. 36(4): p. 357-361.
Jonas, E. A., Hickman, J. A., Hardwick, J. M., Kaczmarek, L. K., Exposure to hypoxia rapidly induces mitochondrial channel activity within a living synapse. The Journal of biological chemistry, 2005. 280(6): p. 4491-4497.
Katz, B. and Miledi, R., A study of synaptic transmission in the absence of nerve impulses. The Journal of physiology, 1967. 192(2): p. 407-436.
Katz, B. and Miledi, R., The effect of prolonged depolarization on synaptic transfer in the stellate ganglion of the squid. The Journal of physiology, 1971. 216(2): p. 503-512.
Khasnavis, S., Jana, A, Roy, A., Mazumder, M., Bhushan, B., Wood, T., Ghosh, S., Watson, R., Pahan, K., Suppression of nuclear factor-B Activation and inflammation in microglia by physically modified saline., J. Biol. Chem., 2012, 287(35), 29529-29542.
Kusano, K., Further study of the relationship between pre- and postsynaptic potentials in the squid giant synapse. The Journal of general physiology, 1968. 52(2): p. 326-45.
Kusano, K. and Landau, E. M., Depression and recovery of transmission at the squid giant synapse. The Journal of physiology, 1975. 245(1): p. 13-32.
Lardy, H. A., Johnson D., McMurray, W. C., Antibiotics as tools for metabolic studies. I. A survey of toxic antibiotics in respiratory, phosphorylative and glycolytic systems. Arch Biochem Biophys, 1958. 78:587-597.
Lin, J. W., Sugimori, M., Llinas, R. R., McGuinness T. L., Greengard, P., Effect of synapsin I and calcium/calmodulin-dependent protein kinase II on spontaneous neurotransmitter release in the squid giant synapse. PNAS. 1990, 87(21), 8257-8261.
Llinas, R., Joyner, R. W., and Nicholson, C., Equilibrium potential for the postsynaptic response in the squid giant synapse. The Journal of general physiology, 1974. 64: 519-535.
Llinas, R., Steinberg, I. Z. and Walton, K., Presynaptic calcium currents and their relation to synaptic transmission: voltage clamp study in squid giant synapse and theoretical model for the calcium gate. PNAS, 1976. 73(8): p. 2918-2922.
Llinas, R., Steinberg, I. Z., and Walton, K., Relationship between presynaptic calcium current and postsynaptic potential in squid giant synapse. Biophysical journal, 1981. 33(3): p. 323-351.
Llinas, R., Sugimori M., Lin, J. W., Leopold, P. and Brady, S., ATP-dependent directional movement of rat synaptic vesicles injected into the presynaptic terminal of squid giant synapse. PNAS, 1989. 86(14): p. 5656-60.
Llinás, R., Sugimori, M., Lin, J. W., Leopold, P. and Brady, S. ATP-Dependent directional movement of rat synaptic vesicles injected into the presynaptic terminal of squid giant synapse. PNAS, 1989, Vol. 86, p 5656-5660.
Mann, D. W. and Joyner, R. W., Miniature synaptic potentials at the squid giant synapse. Journal of neurobiology, 1978. 9(4): p. 329-335.
Mayor, S., Pagano, R. E., Pathways of clathrin-independent endocytosis., Nat Rev Mol Cell Biol., 2007, 8(8), 603-612.
McElroy, W. D., The Energy Source for Bioluminescence in Isolated System. PNAS 1947. vol. 33, p 342-345.
Mikoshiba, K., Fukuda, M., Moriera, J. E., Lewis, F. M., Sugimori, M., Niinobe, M., Llinas, R., Role of C2A domain of synaptotagmin in transmitter release as determined by specific antibody injection into the squid giant synapse preterminal., PNAS, 1995, 92(23), 10703-10707.
Miledi, R., Miniature synaptic potentials in squid nerve cells. Nature, 1966. 212(5067): p. 1240-1242.
Mondal, S., Martinson, J. A., Ghosh, S., Watson, R., Pahan, K., Protection of Tregs, suppression of Th1 and Th17 cells, and amelioration of experimental allergic encephalomyelitis by physically—Modified saline, PLoS One, 2012, 7(12) e51869.
Morgan, J. R., Prasad, K., Hao, W., Augustine, G. J., Lafer, E. M., A conserved clathrin assembly motif essential for synaptic vesicle endocytosis. The Journal of neuroscience: the official journal of the Society for Neuroscience, 2000. 20(23): p. 8667-8676.
Palay, S. L., Synapses in the central nervous system., J Biophys Biochem Cytol., 1956, 2 (4 Suppl), 193-202.
Pivovarova, N. B., Hogpaisan, J., Andrews, S. B., Friel, D. D., Depolization-induced mitochondrial Ca accumulation in sympathetic neurons: Spatial and temporal characteristics., J Neurosci., 1999, 19(15), 6372-6384.
Pothos, E. N., Larsen, K. E., Krantz, D. E., Liu, Y., Haycock, J. W., Setlik, W., Gershon, M. D., Edwards, R. H., and Sulzer D., Synaptic Vesicle Transporter Expression Regulates Vesicle Phenotype and Quantal Size. The Journal of Neuroscience, 2000, 20(19):7297-7306.
Sahehi Y and De Camili P Synapic Vesicle Endocytosis, Cold Spring Harbour Perspectives in Biology 1-30 2012.
Talbot, J. D., David, G., Barrett, E. F., Inhibition of mitochondrial Ca²⁺ uptake affects phasic release from motor terminals differently depending on external Ca²⁺ J Neurophysiol. 2003. 90:491-502.
Takeuchi, A. and Takeuchi, N. Electrical changes in pre and postsynaptic axons of the giant synapse of Loligo, J. Gen. Physiol. 1962, 45: 1181-1193.
Verveen, A. A. and De Felice, L. J. Membrane Noise. Prog Biophys Mol Biol 1974, 28: 189-265.
Verstreken, P., Ly, C. V., Venken, K. J., Koh, T. W. Zhou, Y., Bellen, H. J., Synaptic mitochondria are critical for mobilization of reserve pool vesicles at Drosophila neuromusclar junctions., Neuron, 2005, 47(3), 365-378.
Vos, M., Lauwers, E., Verstreken, P., Synaptic mitochondria in synaptic transmission and organization of versicle pools in health and disease. Front. Synaptic Neurosi., 2010, 2: 139.
Weight, F. F. and Erulkar, S. D., Modulation of synaptic transmitter release by repetitive postsynaptic action potentials. Science, 1976. 193(4257): p. 1023-1025.

### Incorporation by Reference.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

It should be understood that the drawings and detailed description herein are to be regarded in an illustrative rather than a restrictive manner, and are not intended to limit the invention to the particular forms and examples disclosed. On the contrary, the invention includes any further modifications, changes, rearrangements, substitutions, alternatives, design choices, and embodiments apparent to those of ordinary skill in the art, without departing from the spirit and scope of this invention, as defined by the following claims. Thus, it is intended that the following claims be interpreted to embrace all such further modifications, changes, rearrangements, substitutions, alternatives, design choices, and embodiments.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Accordingly, the invention is not limited except as by the appended claims.

1. A method for enhancing hippocampal-mediated learning and memory, comprising administering to a subject in need thereof a therapeutically effective amount of an ionic aqueous solution of charge-stabilized oxygen-containing nanostructures having an average diameter of less than 100 nanometers for enhancing hippocampal-mediated learning and memory in the subject.

2. The method of claim 1, wherein the ionic aqueous solution comprises dissolved oxygen in an amount selected from the group of at least 8 ppm, at least 15 ppm, at least 25 ppm, at least 30 ppm, at least 40 ppm, at least 50 ppm, and at least 60 ppm oxygen at atmospheric pressure and ambient temperature.

3. The method of claim 1, wherein the percentage of dissolved oxygen molecules present in the solution as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%; 60%; 65%; 70%; 75%; 80%; 85%; 90%; and 95% at atmospheric pressure and ambient temperature.

4. The method of claim 3, wherein the amount of dissolved oxygen present in charge-stabilized oxygen-containing nanostructures is an amount selected from the group consisting of at least 8 ppm, at least 15, ppm, at least 20 ppm, at least 25 ppm, at least 30 ppm, at least 40 ppm, at least 50 ppm, and at least 60 ppm oxygen at atmospheric pressure and ambient temperature.

5. The method of claim 3, wherein the majority of the dissolved oxygen is present in the charge-stabilized oxygen-containing nanostructures.

6. The method of claim 1, wherein the charge-stabilized oxygen-containing nanostructures have an average diameter of less than a size selected from the group consisting of: 90 nm; 80 nm; 70 nm; 60 nm; 50 nm; 40 nm; 30 nm; 20 nm; 10 nm; and less than 5 nm.

7. The method of claim 1, wherein the ionic aqueous solution comprises a water or saline solution.

8. The method of claim 1, wherein the solution is superoxygenated.

9. The method of claim 1, wherein the charge-stabilized oxygen-containing nanostructures comprise charge-stabilized oxygen-containing nanobubbles having an average diameter of less than 100 nanometers.

10. The method of claim 1, comprising modulation of at least one of cellular membrane potential and cellular membrane conductivity in hippocampal cells of the subject.

11. The method of claim 1, wherein enhancing learning and/or memory, comprises enhancing learning and/or memory in at least one group selected from the group consisting of normal subjects, subject recovering from neurological trauma, and subjects with learning disorders.

12. The method of claim 11, wherein the learning disorder comprises one selected from the group consisting of: dyslexia, dyscalculia, dysgraphia, dyspraxia (sensory integration disorder), dysphasia/aphasia, auditory processing disorder, non-verbal learning disorder, visual processing disorder, and attention deficit disorder (ADD).

13. The method of claim 11, wherein neurological trauma comprises at least one of accidents or injury to the brain, stroke, oxygen deprivation, drowning, and asphyxia.

14. The method of claim 1, wherein administration promotes modulating or upregulating, in hippocampal neurons, of expression, amount or activity levels of at least one neuronal plasticity protein selected from the group consisting of NR2A and/or NR2B subunits NMDA receptors, GluR1 (glur1) subunit of AMPA receptors, Arc (arc), PSD95, CREB (creb): IEGs including arc, zif-268, and c-fos; NMDA receptor subunits including nr1, nr2a, nr2b, and nr2c; AMPA receptor subunit glur1; neurotrophic factors and their receptors including bdnf, nt3, nt5, and ntrk2; adenylate cyclases (adcy1 and adcy8); camk2a, akt1; ADAM-10, Synpo and homer-1.

15. The method of claim 1, wherein administration promotes modulating or downregulating expression, amount or activity levels of at least one protein selected from the group consisting of Gria2, Ppp1ca, Ppp2ca, and Ppp3ca, proteins encoded by genes known to support long-term depression.

16. The method of claim 1, comprising combination therapy, wherein at least one additional therapeutic agent is administered to the patient.

17. The method of claim 16, wherein, the at least one additional therapeutic agent is selected from the group consisting of: glatiramer acetate, interferon-β, mitoxantrone, natalizumab, inhibitors of MMPs including inhibitor of MMP-9 and MMP-2, short-acting β₂-agonists, long-acting β₂-agonists, anticholinergics, corticosteroids, systemic corticosteroids, mast cell stabilizers, leukotriene modifiers, methylxanthines, β₂-agonists, albuterol, levalbuterol, pirbuterol, artformoterol, formoterol, salmeterol, anticholinergics including ipratropium and tiotropium; corticosteroids including beclomethasone, budesonide, flunisolide, fluticasone, mometasone, triamcinolone, methyprednisolone, prednisolone, prednisone; leukotriene modifiers including montelukast, zafirlukast, and zileuton; mast cell stabilizers including cromolyn and nedocromil; methylxanthines including theophylline; combination drugs including ipratropium and albuterol, fluticasone and salmeterol, budesonide and formoterol; antihistamines including hydroxyzine, diphenhydramine, loratadine, cetirizine, and hydrocortisone; immune system modulating drugs including tacrolimus and pimecrolimus; cyclosporine; azathioprine; mycophenolatemofetil; and combinations thereof.

18. The method of claim 16, wherein the at least one additional therapeutic agent is an anti-inflammatory agent.

19. The method of claim 10, wherein modulation of at least one of cellular membrane potential and cellular membrane conductivity comprises modulating at least one of cellular membrane structure or function comprising modulation of at least one of an amount, conformation, activity, ligand binding activity and/or a catalytic activity of a membrane associated protein.

20. The method of claim 19, wherein the membrane associated protein comprises at least one selected from the group consisting of receptors, ion channel proteins, intracellular attachment proteins, cellular adhesion proteins, and integrins.

21. The method of claim 20, wherein the receptor comprises a transmembrane receptor.

22. The method of claim 10, wherein modulating cellular membrane conductivity comprises modulating whole-cell conductance.

23. The method of claim 22, wherein modulating whole-cell conductance comprises modulating at least one voltage-dependent contribution of the whole-cell conductance.

24. The method of claim 10, wherein modulation of at least one of cellular membrane potential and cellular membrane conductivity comprises modulating a calcium dependent cellular messaging pathway or system.

25. The method of claim 24, comprising modulating calcium influx through ionotropic glutamate receptors.

26. The method of claim 25, wherein the ionotropic glutamate receptor comprises at least one NMDA and/or AMPA receptor.

27. The method of claim 10, wherein modulation of at least one of cellular membrane potential and cellular membrane conductivity comprises modulating intracellular signal transduction comprising modulation of phospholipase C activity or modulation of adenylate cyclase (AC) activity.

28. (canceled)

29. The method of claim 1, comprising administration to a cell network or layer, and further comprising modulation of an intercellular junction therein.

30. The method of claim 10, wherein the ability of the fluid to modulate of at least one of cellular membrane potential and cellular membrane conductivity persists for a time period selected from the group consisting of at least two, at least three, at least four, at least five, at least 6, and at least 12 months, in a closed gas-tight container.

31. The method of claim 1, wherein treating comprises administration by at least one of topical, inhalation, intranasal, oral, intravenous (IV) and intraperitoneal (IP).

32. The method of claim 1, wherein the charge-stabilized oxygen-containing nanostructures are formed in a solution comprising at least one salt or ion from Tables 1 and 2 disclosed herein.

33. The method of claim 1, wherein the subject is a mammal, preferably a human.

34. The method of claim 1, further comprising enhancing the synaptic maturation of neurons by enriching the density and size of dendritic spines.

35. The method of claim 1, further comprising modulating at least one presynaptic and/or postsynaptic response, wherein optimizing or enhancing neuronal synaptic transmission is afforded.

36. The method of claim 35, further comprising enhancing intracellular oxygen delivery or utilization.

37. The method of claim 35, further comprising comprises an increase in ATP synthesis.

38. A method for enhancing the synaptic maturation of neurons by enriching the density and size of dendritic spines, comprising administering to a neuron or subject in need thereof a therapeutically effective amount of an ionic aqueous solution of charge-stabilized oxygen-containing nanostructures having an average diameter of less than 100 nanometers sufficient for enhancing the synaptic maturation of neurons by enriching the density and size of dendritic spines.

39. The method of claim 38, comprising enhancing at least one of the length of primary axons, the number of collaterals, or the number of tertiary branches.

40. The method of claim 38, wherein the ionic aqueous solution comprises dissolved oxygen in an amount selected from the group consisting of at least 8 ppm, at least 15 ppm, at least 25 ppm, at least 30 ppm, at least 40 ppm, at least 50 ppm, and at least 60 ppm oxygen at atmospheric pressure and ambient temperature.

41. The method of claim 38, wherein the percentage of dissolved oxygen molecules present in the solution as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%; 60%; 65%; 70%; 75%; 80%; 85%; 90%; and 95% at atmospheric pressure and ambient temperature.

42. The method of claim 38, wherein the amount of dissolved oxygen present in charge-stabilized oxygen-containing nanostructures is an amount selected from the group consisting of at least 8 ppm, at least 15 ppm, at least 20 ppm, at least 25 ppm, at least 30 ppm, at least 40 ppm, at least 50 ppm, and at least 60 ppm oxygen at atmospheric pressure and ambient temperature.

43. The method of claim 38, wherein the majority of the dissolved oxygen is present in the charge-stabilized oxygen-containing nanostructures.

44. The method of claim 38, wherein the charge-stabilized oxygen-containing nanostructures have an average diameter of less than a size selected from the group consisting of: 90 nm; 80 nm; 70 nm; 60 nm; 50 nm; 40 nm; 30 nm; 20 nm; 10 nm; and less than 5 nm.

45. The method of claim 38, wherein the ionic aqueous solution comprises a water or saline solution.

46. The method of claim 38, wherein the solution is superoxygenated.

47. The method of claim 38, wherein the charge-stabilized oxygen-containing nanostructures comprise charge-stabilized oxygen-containing nanobubbles having an average diameter of less than 100 nanometers.

48. The method of claim 38, wherein the neurons are hippocampal neurons.

49. The method of claim 38, further comprising modulating at least one presynaptic and/or postsynaptic response, wherein optimizing or enhancing neuronal synaptic transmission is afforded.

50. A method for maintaining, growing or enhancing the synaptic maturation of neurons in culture, comprising administering to a neuron in need thereof an effective amount of an ionic aqueous solution of charge-stabilized oxygen-containing nanostructures having an average diameter of less than 100 nanometers sufficient for maintaining, growing or enhancing the synaptic maturation of neurons in culture.

51. The method of claim 50, wherein the neurons are hippocampal neurons.

52. The method of claim 50, further comprising enriching the density and size of dendritic spines.

53. The method of claim 50, further comprising modulating at least one presynaptic and/or postsynaptic response, wherein optimizing or enhancing neuronal synaptic transmission is afforded.

54. A method for optimizing or enhancing neurotransmission, comprising contacting neurons with, or administrating to a subject having neurons, an electrokinetically-altered ionic aqueous solution comprising charge-stabilized oxygen-containing nanostructures having an average diameter of less than 100 nm in an amount and for a time period sufficient for modulating at least one presynaptic and/or postsynaptic response, wherein a method for optimizing or enhancing neuronal synaptic transmission is afforded.

55. The method of claim 54, wherein modulating at least one presynaptic and/or postsynaptic response comprises an increase of spontaneous transmitter release.

56. The method of claim 54, wherein modulating at least one presynaptic and/or postsynaptic response comprises a modification of noise kinetics.

57. The method of claim 54, wherein modulating at least one presynaptic and/or postsynaptic response comprises an increase in a postsynaptic response.

58. The method of claim 57, comprising an increase in the postsynaptic response without an increase in presynaptic ICa⁺⁺ amplitude.

59. The method of claim 54, wherein modulating at least one presynaptic and/or postsynaptic response comprises a decrease in synaptic vesicle density and/or number at active zones.

60. The method of claim 59, further comprising an increase in the number of clathrin-coated vesicles, and large endosome like vesicles in the vicinity of the junctional sites.

61. The method of claim 54, wherein modulating at least one presynaptic and/or postsynaptic response comprises a marked increase in ATP synthesis leading to synaptic transmission optimization.

62. The method of claim 54, wherein modulating at least one presynaptic and/or postsynaptic response comprises an enhanced or more vigorous recovery of postsynaptic spike generation.

63. The method of claim 54, wherein modulating at least one presynaptic and/or postsynaptic response comprises increased ATP synthesis at the presynaptic and postsynaptic terminals.

64. The method of claim 54, further comprising enhancing intracellular oxygen delivery or utilization.

65. The method of claim 54, wherein the charge-stabilized oxygen-containing nanostructures having an average diameter of less than 100 nm comprise charge-stabilized oxygen-containing nanobubbles having an average diameter of less than 100 nm.

66. A method for optimizing or enhancing neurotransmission, comprising contacting neurons with, or administrating to a subject having neurons, an electrokinetically-altered ionic aqueous solution comprising charge-stabilized oxygen-containing nanostructures having an average diameter of less than 100 nm in an amount and for a time period sufficient for enhancing intracellular oxygen delivery or utilization, wherein a method for optimizing or enhancing neuronal synaptic transmission is afforded.

67. The method of claim 66, wherein optimizing or enhancing neuronal synaptic transmission comprises an increase of spontaneous transmitter release.

68. The method of claim 66, wherein optimizing or enhancing neuronal synaptic transmission comprises a modification of noise kinetics.

69. The method of claim 66, wherein optimizing or enhancing neuronal synaptic transmission comprises an increase in a postsynaptic response.

70. The method of claim 69, comprising an increase in the postsynaptic response without an increase in presynaptic ICa⁺⁺ amplitude.

71. The method of claim 66, wherein optimizing or enhancing neuronal synaptic transmission comprises a decrease in synaptic vesicle density and/or number at active zones.

72. The method of claim 71, further comprising an increase in the number of clathrin-coated vesicles, and large endosome like vesicles in the vicinity of the junctional sites.

73. The method of claim 66, wherein optimizing or enhancing neuronal synaptic transmission comprises a marked increase in ATP synthesis.

74. The method of claim 66, wherein optimizing or enhancing neuronal synaptic transmission comprises an enhanced or more vigorous recovery of postsynaptic spike generation.

75. The method of claim 66, wherein optimizing or enhancing neuronal synaptic transmission comprises increased ATP synthesis at the presynaptic and postsynaptic terminals.

76. The method of claim 66, wherein the charge-stabilized oxygen-containing nanostructures having an average diameter of less than 100 nm comprise charge-stabilized oxygen-containing nanobubbles having an average diameter of less than 100 nm.

77. A method for enhancing intracellular oxygen delivery or utilization, comprising contacting cells with, or administrating to a subject having cells, an electrokinetically-altered ionic aqueous solution comprising charge-stabilized oxygen-containing nanostructures having an average diameter of less than 100 nm in an amount and for a time period sufficient for enhancing intracellular oxygen delivery or utilization in the cells.

78. The method of claim 77, wherein the cells are nerve cells.

79. The method of claim 78, wherein enhancing intracellular oxygen delivery or utilization provides for optimizing or enhancing neuronal synaptic transmission.

80. The method of claim 79, wherein optimizing or enhancing neuronal synaptic transmission comprises an increase of spontaneous transmitter release.

81. The method of claim 79, wherein optimizing or enhancing neuronal synaptic transmission comprises a modification of noise kinetics.

82. The method of claim 79, wherein optimizing or enhancing neuronal synaptic transmission comprises an increase in a postsynaptic response.

83. The method of claim 82, comprising an increase in the postsynaptic response without an increase in presynaptic ICa⁺⁺ amplitude.

84. The method of claim 79, wherein optimizing or enhancing neuronal synaptic transmission comprises a decrease in synaptic vesicle density and/or number at active zones.

85. The method of claim 84, further comprising an increase in the number of clathrin-coated vesicles, and large endosome like vesicles in the vicinity of the junctional sites.

86. The method of claim 79, wherein optimizing or enhancing neuronal synaptic transmission comprises an increase in ATP synthesis.

87. The method of claim 79, wherein optimizing or enhancing neuronal synaptic transmission comprises an enhanced or more vigorous recovery of postsynaptic spike generation.

88. The method of claim 79, wherein optimizing or enhancing neuronal synaptic transmission comprises increased ATP synthesis at the presynaptic and postsynaptic terminals.

89. The method of claim 77, wherein the charge-stabilized oxygen-containing nanostructures having an average diameter of less than 100 nm comprise charge-stabilized oxygen-containing nanobubbles having an average diameter of less than 100 nm.
